SEQUENCE LISTING (349 sequences)

SEQ ID NO: 1 - protein, 18 aa, Artificial Sequence
  CcmN Cterm (Synpcc7942_1424)  YP_400441
VYGKEQFLRMRQSMFPDR

SEQ ID NO: 2 - protein, 13 aa, Artificial Sequence
  CcaA (Synpcc7942_1447)  YP_400464
LAPEQQQRIYRGN

SEQ ID NO: 3 - protein, 16 aa, Artificial Sequence
  EutC Nterm (STM2457) NP_461392
MDQKQIEEIVRSVMAS

SEQ ID NO: 4 - protein, 17 aa, Artificial Sequence
  EutE Nterm (STM2463)  NP_461398
MNQQDIEQVVKAVLLKM

SEQ ID NO: 5 - protein, 16 aa, Artificial Sequence
  EutE (Cphy_2642) YP_001559742
NTELVEEIVKRIMKQL

SEQ ID NO: 6 - protein, 19 aa, Artificial Sequence
  PduD Nterm (STM2041) NP_460986
MEINEKLLRQIIEDVLRDM

SEQ ID NO: 7 - protein, 19 aa, Artificial Sequence
  PduE Nterm (STM2042) NP_460987
MNTDAIESMVRDVLSRMNS

SEQ ID NO: 8 - protein, 16 aa, Artificial Sequence
  PduP Nterm (STM2051) NP_460996
MNTSELETLIRTILSE

SEQ ID NO: 9 - protein, 26 aa, Artificial Sequence
  Putative B12-independent propanediol dehydratase inter-domain (RPC_1163)  YP_531045
AGTNYTEEQVFAAVKKVLNSSGSTDV

SEQ ID NO: 10 - protein, 19 aa, Artificial Sequence
  Aldehyde dehydrogenase Nterm (RPC_1174) YP_531056
MVAKAIRDHAGTAQPSGNA

SEQ ID NO: 11 - protein, 21 aa, Artificial Sequence
  Putative B12-independeent propanediol dehydratase inter-domain (Cphy_1174) YP_001558291
IDIILAQQITVQIVKELKERG

SEQ ID NO: 12 - protein, 18 aa, Artificial Sequence
  Fuculose-phosphate aldolase Cterm (Cphy_1177) YP_001558294
DNADLVASITRKVMEQLG

SEQ ID NO: 13 - protein, 20 aa, Artificial Sequence
  Aldehyde dehydrogenase (Cphy_1178) Nterm YP_001558295
VNEQLVQDIIKNVVASMQLT

SEQ ID NO: 14 - protein, 21 aa, Artificial Sequence
  Aldehyde dehydrogenases Cterm   (Ckl_1074) (Ckl_1076) YP_001394464 YP_001394466
EPEDNEDVQAIVKAIMAKLNL

SEQ ID NO: 15 - protein, 19 aa, Artificial Sequence
  Aldolase Cterm (Plim_1747)
DTEMLVKMITEQVMAALKK

SEQ ID NO: 16 - protein, 20 aa, Artificial Sequence
  Aldehyde dehydrogenase Nterm (Plim_1751)
MQATEQAIRQVVQEVLAQLN

SEQ ID NO: 17 - protein, 18 aa, Artificial Sequence
  Aldolase Cterm (Oter_1298)  YP_001818183
EVEALVQRLTEEILRQLQ

SEQ ID NO: 18 - protein, 17 aa, Artificial Sequence
  Aldehyde dehydrogenase (Oter_1295) YP_001818180
IDETLVRSVVEEVVRAF

SEQ ID NO: 19 - protein, 15 aa, Artificial Sequence
  Aldehyde dehydrogenase I  (Cphy_1416) Cterm Aldehyde dehydrogenase II    (Cphy_1428)  Cterm YP_001558530 YP_001558542
EDARDLLKQILQALS

SEQ ID NO: 20 - protein, 17 aa, Artificial Sequence
  Unknown glycyl radical enzyme Nterm (Cphy_1417) YP_001558531
MDIREFSNKFVEATKNM

SEQ ID NO: 21 - protein, 22 aa, Artificial Sequence
  Aldehyde dehydrogenase Cterm (MSMEG_0276) YP_884691
LDALRAELRALVVEELAQLIKR

SEQ ID NO: 22 - protein, 19 aa, Artificial Sequence
  Aldehyde dehydrogenase Nterm (HochDRAFT_00990) ZP_03875711
MALREDRIAEIVERVLARL

SEQ ID NO: 23 - protein, 18 aa, Artificial Sequence
  Bacterial microcompartments-associated metabolic pathway: Calvin cycle
  PEPTIDE (1)..(1): X= (V/I)
  PEPTIDE (2)..(2): X= (V/Y)
  PEPTIDE (4)..(4): X= (Q/K)
  PEPTIDE (5)..(5): X= (V/A/G/E)
  PEPTIDE (6)..(6): X= (Y/S/Q)
  PEPTIDE (7)..(7): X= (I/V/L/F)
  PEPTIDE (8)..(8): X= (N/Q/S/L)
  PEPTIDE (9)..(9): X= (K/Q/R)
  PEPTIDE (10)..(10): X= (M/L)
  PEPTIDE (11)..(11): X= (L/M/R)
  PEPTIDE (12)..(12): X= (V/L/C/Q)
  PEPTIDE (13)..(13): X= (T/S)
  PEPTIDE (14)..(14): X= (L/M)
  PEPTIDE (17)..(17): X= (H/D/E)
  PEPTIDE (18)..(18): X= (R/N/Q)
XXGXXXXXXXXXXXFPXX

SEQ ID NO: 24 - protein, 13 aa, Artificial Sequence
  Bacterial microcompartments-associated metabolic pathway: Calvin cycle
  PEPTIDE (1)..(1): X= (L/F)
  PEPTIDE (2)..(2): X= (S/P/A)
  PEPTIDE (3)..(3): X= (P/V)
  PEPTIDE (4)..(4): X= (E/Q)
  PEPTIDE (6)..(6): X= (A/S/Q/W)
  PEPTIDE (7)..(7): X= (Q/E/R)
  PEPTIDE (11)..(11): X= (R/Q)
  PEPTIDE (13)..(13): X= (S/N)
XXXXQXXRIYXGX

SEQ ID NO: 25 - protein, 16 aa, Artificial Sequence
  Bacterial microcompartments-associated metabolic pathway: Ethanolamine utilization
  PEPTIDE (2)..(2): X= (D/N)
  PEPTIDE (3)..(3): X= (E/Q)
  PEPTIDE (4)..(4): X= (K/Q)
  PEPTIDE (5)..(5): X= (Q/E)
  PEPTIDE (6)..(6): X= (L/I)
  PEPTIDE (7)..(7): X= (K/R/E)
  PEPTIDE (8)..(8): X= (E/D)
  PEPTIDE (9)..(9): X= (I/M)
  PEPTIDE (10)..(10): X= (V/I)
  PEPTIDE (11)..(11): X= (R/E)
  PEPTIDE (12)..(12): X= (S/Q)
  PEPTIDE (13)..(13): X= (V/I)
  PEPTIDE (14)..(14): X= (L/M)
  PEPTIDE (16)..(16): X= (E/Q/S)
MXXXXXXXXXXXXXAX

SEQ ID NO: 26 - protein, 17 aa, Artificial Sequence
  Bacterial microcompartments-associated metabolic pathway: Ethanolamine utilization
MNQQDIEQVVKAVLLKM

SEQ ID NO: 27 - protein, 16 aa, Artificial Sequence
  Bacterial microcompartments-associated metabolic pathway: Ethanolamine utilization
  PEPTIDE (1)..(1): X= (A/K/S)
  PEPTIDE (2)..(2): X= (E/D)
  PEPTIDE (3)..(3): X= (A/E)
  PEPTIDE (5)..(5): X= (I/V)
  PEPTIDE (6)..(6): X= (E/D/N)
  PEPTIDE (7)..(7): X= (L/E/S)
  PEPTIDE (8)..(8): X= (I/L)
  PEPTIDE (9)..(9): X= (V/I)
  PEPTIDE (10)..(10): X= (R/K/E/Q)
  PEPTIDE (11)..(11): X= (K/R)
  PEPTIDE (14)..(14): X= (E/A)
  PEPTIDE (15)..(15): X= (E/K)
XXXLXXXXXXXVLXXL

SEQ ID NO: 28 - protein, 19 aa, Artificial Sequence
  Bacterial microcompartments-associated metabolic pathway: Propanediol utilization (B12 dependent)
  PEPTIDE (4)..(4): X= (N/D/T)
  PEPTIDE (6)..(6): X= (K/E)
  PEPTIDE (7)..(7): X= (L/V)
  PEPTIDE (8)..(8): X= (L/V)
  PEPTIDE (9)..(9): X= (R/E)
  PEPTIDE (11)..(11): X= (I/V)
  PEPTIDE (12)..(12): X= (I/V)
  PEPTIDE (13)..(13): X= (E/K/A)
  PEPTIDE (14)..(14): X= (D/E)
  PEPTIDE (17)..(17): X= (K/S/R/A)
  PEPTIDE (18)..(18): X= (E/D)
  PEPTIDE (19)..(19): X= (M/L)
MEIXEXXXXQXXXXVLXXX

SEQ ID NO: 29 - protein, 18 aa, Artificial Sequence
  Bacterial microcompartments-associated metabolic pathway: Propanediol utilization (B12 dependent)
  PEPTIDE (1)..(1): X= (M/I)
  PEPTIDE (2)..(2): X= (N/D)
  PEPTIDE (3)..(3): X= (T/E)
  PEPTIDE (4)..(4): X= (D/K)
  PEPTIDE (5)..(5): X= (A/L)
  PEPTIDE (6)..(6): X= (I/L)
  PEPTIDE (8)..(8): X= (S/E)
  PEPTIDE (9)..(9): X= (M/I)
  PEPTIDE (11)..(11): X= (R/K)
  PEPTIDE (12)..(12): X= (D/E/Q)
  PEPTIDE (15)..(15): X= (S,N)
  PEPTIDE (16)..(16): X= (M/L)
  PEPTIDE (17)..(17): X= (N/E/G)
XXXXXXEXXVXXVLXXXS

SEQ ID NO: 30 - protein, 16 aa, Artificial Sequence
  Bacterial microcompartments-associated metabolic pathway: Propanediol utilization (B12 dependent)
  PEPTIDE (2)..(2): X= (N/D/E)
  PEPTIDE (3)..(3): X= (T/S/E)
  PEPTIDE (4)..(4): X= (S/L)
  PEPTIDE (6)..(6): X= (L/V)
  PEPTIDE (8)..(8): X= (T/Q/K/D)
  PEPTIDE (9)..(9): X= (L/I)
  PEPTIDE (10)..(10): X= (I/V)
  PEPTIDE (11)..(11): X= (R/K)
  PEPTIDE (12)..(12): X= (T/N/K)
  PEPTIDE (13)..(13): X= (I/V)
  PEPTIDE (14)..(14): X= (L/I)
  PEPTIDE (15)..(15): X= (S/L/R/N)
MXXXEXEXXXXXXXXE

SEQ ID NO: 31 - protein, 26 aa, Artificial Sequence
  Bacterial microcompartments-associated metabolic pathway: 1,2-propanediol utilization (B12 independent) (putative)
  PEPTIDE (1)..(1): X= (A/P)
  PEPTIDE (2)..(2): X= (K/G)
  PEPTIDE (3)..(3): X= (S/Q)
  PEPTIDE (4)..(4): X= (S/D)
  PEPTIDE (5)..(5): X= (L/A)
  PEPTIDE (6)..(6): X= (T/N)
  PEPTIDE (8)..(8): X= (E/Q)
  PEPTIDE (9)..(9): X= (D/Q)
  PEPTIDE (10)..(10): X= (I/V)
  PEPTIDE (12)..(12): X= (D/E)
  PEPTIDE (16)..(16): X= (K/R)
  PEPTIDE (17)..(17): X= (V/I)
  PEPTIDE (18)..(18): X= (L/I)
  PEPTIDE (19)..(19): X= (E/G)
  PEPTIDE (20)..(20): X= (Q/E/S)
  PEPTIDE (21)..(21): X= (H/S)
  PEPTIDE (23)..(23): X= (A/S)
  PEPTIDE (26)..(26): X= (P/V)
XXXXXXEXXXYXAVKXXXXXXGXLDX

SEQ ID NO: 32 - protein, 19 aa, Artificial Sequence
  Bacterial microcompartments-associated metabolic pathway: 1,2-propanediol utilization (B12 independent) (putative)
  PEPTIDE (3)..(3): X= (D/T)
  PEPTIDE (4)..(4): X= (I/T)
  PEPTIDE (5)..(5): X= (E/Q)
  PEPTIDE (6)..(6): X= (I/L)
  PEPTIDE (7)..(7): X= (A/E)
  PEPTIDE (8)..(8): X= (Q/N)
  PEPTIDE (9)..(9): X= (A/M)
  PEPTIDE (10)..(10): X= (V/I)
  PEPTIDE (11)..(11): X= (S/R/A)
  PEPTIDE (12)..(12): X= (T/K/N)
  PEPTIDE (15)..(15): X= (S/A/E/R)
  PEPTIDE (16)..(16): X= (D/K)
  PEPTIDE (17)..(17): X= (N/F/Y)
  PEPTIDE (18)..(18): X= (T/L/G)
MNXXXXXXXXXXILXXXXK

SEQ ID NO: 33 - protein, 21 aa, Artificial Sequence
  Bacterial microcompartments-associated metabolic pathway: Dissimilation of fucose and rhamnose to primary alcohols (putative)
  PEPTIDE (3)..(3): X= (A/E)
  PEPTIDE (6)..(6): X= (A/V)
  PEPTIDE (7)..(7): X= (A/G)
  PEPTIDE (9)..(9): X= (M/I)
  PEPTIDE (10)..(10): X= (T/A)
  PEPTIDE (12)..(12): X= (M/Q)
  PEPTIDE (14)..(14): X= (A/L)
  PEPTIDE (16)..(16): X= (E/G)
  PEPTIDE (17)..(17): X= (L/M)
  PEPTIDE (18)..(18): X= (K/Q)
  PEPTIDE (19)..(19): X= (E/D)
LDXESXXDXXEXIXKXXXXAG

SEQ ID NO: 34 - protein, 18 aa, Artificial Sequence
  Bacterial microcompartments-associated metabolic pathway: Dissimilation of fucose and rhamnose to primary alcohols (putative)
  PEPTIDE (1)..(1): X= (D/P)
  PEPTIDE (2)..(2): X= (D/N)
  PEPTIDE (3)..(3): X= (A/E)
  PEPTIDE (4)..(4): X= (D/E/A)
  PEPTIDE (6)..(6): X= (V/I)
  PEPTIDE (8)..(8): X= (E/A/S)
  PEPTIDE (11)..(11): X= (K/R)
  PEPTIDE (12)..(12): X= (K/R/Q)
  PEPTIDE (14)..(14): X= (M/L)
  PEPTIDE (15)..(15): X= (A/E)
  PEPTIDE (18)..(18): X= (G/K)
XXXXLXAXITXXVXXQLX

SEQ ID NO: 35 - protein, 20 aa, Artificial Sequence
  Bacterial microcompartments-associated metabolic pathway: Dissimilation of fucose and rhamnose to primary alcohols (putative)
  PEPTIDE (5)..(5): X= (L/M)
  PEPTIDE (11)..(11): X= (Q/R/K)
  PEPTIDE (16)..(16): X= (K/R)
  PEPTIDE (20)..(20): X= (S/T)
VNEQXVQDIVXEVVAXMQIX

SEQ ID NO: 36 - protein, 21 aa, Artificial Sequence
  Bacterial microcompartments-associated metabolic pathway: Ethanol utilization; Aldehyde dehydrogenase Cterm
EPEDNEDVQAIVKAIMAKLNL

SEQ ID NO: 37 - protein, 19 aa, Artificial Sequence
  Bacterial microcompartments-associated metabolic pathway: Fuculose-1-phosphate metabolism (putative)
  PEPTIDE (4)..(4): X= (A/Q)
  PEPTIDE (7)..(7): X= (K/Q)
  PEPTIDE (8)..(8): X= (A/L)
  PEPTIDE (11)..(11): X= (D/E)
  PEPTIDE (12)..(12): X= (Q/R/E)
  PEPTIDE (16)..(16): X= (A/E)
  PEPTIDE (18)..(18): X= (K/S)
DQEXLVXXITXXVMAXLXK

SEQ ID NO: 38 - protein, 20 aa, Artificial Sequence
  Bacterial microcompartments-associated metabolic pathway: Fuculose-1-phosphate metabolism (putative)
  PEPTIDE (3)..(3): X= (I/A)
  PEPTIDE (4)..(4): X= (D/T)
  PEPTIDE (7)..(7): X= (L/A)
  PEPTIDE (13)..(13): X= (A/Q)
  PEPTIDE (14)..(14): X= (Q/E)
  PEPTIDE (17)..(17): X= (A/S)
  PEPTIDE (18)..(18): X= (E/Q)
  PEPTIDE (19)..(19): X= (V/L)
  PEPTIDE (20)..(20): X= (G/N)
MQXXEEXIRSVVXXVLXXXX

SEQ ID NO: 39 - protein, 18 aa, Artificial Sequence
  Bacterial microcompartments-associated metabolic pathway: Fuculose-1-phosphate and rhamnulose-1-phosphate conversion to acetate or pyruvate (putative); Aldolase Cterm
EVEALVQRLTEEILRQLQ

SEQ ID NO: 40 - protein, 17 aa, Artificial Sequence
  Bacterial microcompartments-associated metabolic pathway: Fuculose-1-phosphate and rhamnulose-1-phosphate conversion to acetate or pyruvate (putative); Aldehyde dehydrogenase Nterm
IDETLVRSVVEEVVRAF

SEQ ID NO: 41 - protein, 15 aa, Artificial Sequence
  Bacterial microcompartments-associated metabolic pathway: Unknown glycyl radical enzyme (putative)
  PEPTIDE (1)..(1): X= (E/Q/D)
  PEPTIDE (2)..(2): X= (N/E/D)
  PEPTIDE (3)..(3): X= (V/I/L)
  PEPTIDE (4)..(4): X= (E/Q/A)
  PEPTIDE (5)..(5): X= (R/Q/D)
  PEPTIDE (6)..(6): X= (I/L/V)
  PEPTIDE (7)..(7): X= (I/L/V)
  PEPTIDE (8)..(8): X= (K/R/N)
  PEPTIDE (9)..(9): X= (E/Q/K)
  PEPTIDE (10)..(10): X= (V/I/L)
  PEPTIDE (11)..(11): X= (L/I/V)
  PEPTIDE (12)..(12): X= (E/Q/G)
  PEPTIDE (13)..(13): X= (Q/R/A)
  PEPTIDE (14)..(14): X= (L/M)
  PEPTIDE (15)..(15): X= (K/G/S)
XXXXXXXXXXXXXXX

SEQ ID NO: 42 - protein, 17 aa, Artificial Sequence
  Bacterial microcompartments-associated metabolic pathway: Unknown glycyl radical enzyme (putative)
  PEPTIDE (2)..(2): X= (A/D)
  PEPTIDE (3)..(3): X= (K/I/N/L)
  PEPTIDE (4)..(4): X= (R/Y)
  PEPTIDE (5)..(5): X= (E/N/S)
  PEPTIDE (6)..(6): X= (L/F)
  PEPTIDE (7)..(7): X= (T/S)
  PEPTIDE (8)..(8): X= (P/N)
  PEPTIDE (9)..(9): X= (R/K)
  PEPTIDE (10)..(10): X= (V/L/F)
  PEPTIDE (11)..(11): X= (K/A)
  PEPTIDE (12)..(12): X= (E/V/M)
  PEPTIDE (13)..(13): X= (L/A)
  PEPTIDE (14)..(14): X= (A/T)
  PEPTIDE (15)..(15): X= (E/K)
  PEPTIDE (16)..(16): X= (R/N)
  PEPTIDE (17)..(17): X= (L/M)
MXXXXXXXXXXXXXXXX

SEQ ID NO: 43 - protein, 22 aa, Artificial Sequence
  Bacterial microcompartments-associated metabolic pathway: Arginine or serine/threonine metabolism (putative)
  PEPTIDE (2)..(2): X= (E/D/G)
  PEPTIDE (6)..(6): X= (A/E/D)
  PEPTIDE (10)..(10): X= (A/R)
  PEPTIDE (12)..(12): X= (V/I)
  PEPTIDE (13)..(13): X= (V/A)
  PEPTIDE (17)..(17): X= (A/R)
  PEPTIDE (18)..(18): X= (Q/E))
  PEPTIDE (20)..(20): X= (I/N/G)
  PEPTIDE (21)..(21): X= (K/R)
  PEPTIDE (22)..(22): X= (R/Q)
IXALRXELRXLXXEELXXLXXX

SEQ ID NO: 44 - protein, 19 aa, Artificial Sequence
  Bacterial microcompartments-associated metabolic pathway: Serine-threonine metabolism (putative); Nterm Aldehyde dehydrogenase
MALREDRIAEIVERVLARL

SEQ ID NO: 45 - protein, 17 aa, Artificial Sequence
  Consensus amphipathic helix for targeting bacterial microcompartments
  PEPTIDE (1)..(1): X is I,L,V,M,F,Y,A, or W
  PEPTIDE (2)..(2): X is Q,N,T,S, or C
  PEPTIDE (3)..(3): X is D,E,R,K, or H
  PEPTIDE (4)..(4): X is D,E,R,K, or H
  PEPTIDE (5)..(5): X is any residue
  PEPTIDE (6)..(6): X is I,L,V,M,F,Y,A, or W
  PEPTIDE (7)..(7): X is D,E,R,K, or H
  PEPTIDE (8)..(8): X is Q,N,T,S, or C
  PEPTIDE (9)..(9): X is I,L,V,M,F,Y,A, or W
  PEPTIDE (10)..(10): X is I,L,V,M,F,Y,A, or W
  PEPTIDE (11)..(11): X is D,E,R,K, or H
  PEPTIDE (12)..(12): X is D,E,R,K, or H
  PEPTIDE (13)..(13): X is I,L,V,M,F,Y,A, or W
  PEPTIDE (14)..(14): X is I,L,V,M,F,Y,A, or W
  PEPTIDE (15)..(15): X is any residue
  PEPTIDE (16)..(16): X is D,E,R,K, or H
  PEPTIDE (17)..(17): X is I,L,V,M,F,Y,A, or W
XXXXXXXXXXXXXXXXX

SEQ ID NO: 46 - protein, 18 aa, Artificial Sequence
  Carboxysome protein, CcmN
VYGQVYINQLLQTLFPHR

SEQ ID NO: 47 - protein, 262 aa, Artificial Sequence
  Cyanothece_sp_PCC7822_642884450/1-262
MHLPPVQPVSVSEIYVSGDVIIHDSAVVAPGTILQAAPNSRIVIGAGACIGMGVVLNAYRGEIEIESGAVLGSGVLILGTGKIGKNACVGSLTTLLNSSIEPMAVITAGSLIGDTTRSFTPEPETTNGNGAKQPDFSKLNRPEKIQEELPPIVASPPKEHPSVVELESDPWTIDPIDDDQSSSKSDSVLSNTQVHEPEPATETRVEVTPQPPDLEPTEQSKQAPVVGQIYINQLLLTLFPERRFFQNLDQKNQSLHSEENSQ

SEQ ID NO: 48 - protein, 201 aa, Artificial Sequence
  Gloeobacter_violaceus_637459485/1-201
MASLPPPWDANAYTSGDVTIHPGAAVASGALLRADPDSRIVIGSGACIGMGAILHAHQGTLEVGSGASLGAGVLVVGRGKIGADACVGTATTLLNPDIAPGQVVPPNSLVGQAGRSAEAFPTAAAQPYVVPAAPAPRDPNQALAAGFDPPVQAALPEPQGGIVQNGQPPVAGKAYLERLRLSLFPHNAPLQNPDSATGGGA

SEQ ID NO: 49 - protein, 161 aa, Artificial Sequence
  Synechococcus_elongatus_PCC6301_637615774/1-161
MHLPPLEPPISDRYFASGEVTIAADVVIAPGVLLIAEADSRIEIASGVCIGLGSVIHARGGAIIIQAGALLAAGVLIVGQSIVGRQACLGASTTLVNTSIEAGGVTAPGSLLSAETPPTTATVSSSEPAGRSPQSSAIAHPTKVYGKEQFLRMRQSMFPDR

SEQ ID NO: 50 - protein, 161 aa, Artificial Sequence; identical to SEQ ID NO: 49
  Synechococcus_elongatus_PCC7942_637799856/1-161

SEQ ID NO: 51 - protein, 304 aa, Artificial Sequence
  Trichodesmium_erythraeum_638108779/1-304
MQLPPLQPFANIEPFVSGDVKIDPSAAIAPGVILQAASNCQIIIGAGVCIGMGVIIHAYSGNIEIESGATIGSGVLLVGKSKIGANVCIGSLATILEQNLESEKVVLPASIIGNSGRQFSDNSTISLPDQDSNQSYLFSNETQESSYSLNLANTASSTEETSTETEKANTQLPLANTSLPAEETPTETEKANTQLPLANTSLPAEETPTETEKANTQLPLANTSLPVEETPTETEKANTQLPLANTSLPVEETPTETEKANTQLQEESPPNIDAQIYGKEYVNKIMQTLFPYKNSLSSHPDDED

SEQ ID NO: 52 - protein, 220 aa, Artificial Sequence
  Thermosynechococcus_elongatus_637313560/1-220
MPLPPLALPPSPAVRIVGDVVVDPQAVLAPGVLLWAEAGAAIRIASGVCIGMGCIIHAHGGTIAIGEGVNIGAGVLLIGAVTVEPHACIGASTTVMQTTIPAGAVVAAGSLVGDRSRRWPPAAETSHPQQRTVFPEDPWQEPATTAHTSENSPQQEQEATDSPPNHQESPAAAPPETSTATRPKASVVYGQAYVSKMFAKMFRVAPIPPTGDNSALGSSQ

SEQ ID NO: 53 - protein, 231 aa, Artificial Sequence
  Cyanothece_sp_PCC7425_643584614/1-231
MYLPSPQPLSHGPTSVIGDVQIHPNAVIAPGVLLYAEPDSQITIAAGVCIGMGSILHAHGGKVDVEAGANLGTGVLIVGTARIGSHACIGSTTTIINTDLPPAAVVAPGSLVGDPSRRPPELTETEALQEEQPTHLQPAQSQSDEPQTDQSPAAQEEQGDLQSASPAPVDHAAGTNSSPSPQAEQQTDAPPRSVYGQDYVNRMMQRMMPRTPSLTPSPTGQNGSVEGGTGS

SEQ ID NO: 54 - protein, 224 aa, Artificial Sequence
  Lyngbya_sp_PCC8106_640017143/1-224
MYRSPPQPLNNASAFVSGDVTIDPSVAIAMGVILQADPDSQIVIATGVCIGMGAIIHAYQGKIEVGAGANIGAGVLVVGHGTIGAKACIGAETTLLNPVITAKQVVPAGTIIGDESRSVTLSSSSEEEKNDLGEVQTSPTEKNDPGEVQTSSTDHLNNSQSEESSEVSPETSSVSNSTTATSLEKSPNPTASIVYGQVHLNQLLNTLLPHRRSLNNSNPTDRSP

SEQ ID NO: 55 - protein, 244 aa, Artificial Sequence
  Cyanothece_sp_PCC8802_644979618/1-244
MYLPLIRPATHSDICVIGDVTIHDNAVIAPGTILQAAPGCRILIKEGACIGMGSLLNAYNGDIEVASGAMLGAGVLVVGHSKIGQNACIGSSTTIINSSIDSGTAIAPGSLVGDQSRQVVSETSPSTKEIKSENNGSVANNNGSTFNNDHIASKVASTEDKKPTFVQEMEDLWAEPEPEVEPVAEVSPPPKPSVEPIPEVLTQPKPSPDPQNAPVVGQIYINQLLYTLFPERQAFNRSQNGSSS

SEQ ID NO: 56 - protein, 239 aa, Artificial Sequence
  Crocosphaera_watsonii_638429558/1-240
MPLPLIQPPSRSEVSVIGEVIIHQGAVVAPGTILQAAPNCRIVIHSGACIGMGTLINYQGDIEIESGAMLGAGVLIVGQSKISQNVCLGSCTTVINSSIESGTTIEAGTLIGDTSRQFSEEETKAPKQIKAENNGSSENGHLIADNNQKDNLPQQSEEKKPEFVEEIEDLWADTPPKVEEVTEIPEIPTKPDTPTETKNAPVVGQVYINQLLCTLFPDRQAFNQSQNNSASKDPPGKNK

SEQ ID NO: 57 - protein, 241 aa, Artificial Sequence
  Cyanothece_sp._CCY0110_640626457/1-241
MPLPLIQPPRHSEVSITGEVIIHEGAVVAPGTILQAAPNCRIVIHSGACIGMGTLINAYKGDIEIESGAMLGAGVLIVGHGKIGQNVCLGSCTTVINTSIESGTTIEAGSLMGDTSRQFQEKESQSPPAIKADDNGFGDNGHLTANDQKKASQTDTTNHNKPGFVEEMEDLWADSEPEIEEVTKIPEIPEIPTKSNSPADKNNAPVVGQVYINQLLCTLFPDRQAFNQAQNNPPSQDENNE

SEQ ID NO: 58 - protein, 240 aa, Artificial Sequence
  Cyanothece_sp_ATCC51142_641678787/1-240
MPLPLIQPPSRSEVSIIGEVIIHEGAVVAPGTILQAAPDCRIVIHQGACIGMGTLINAYQGDIEIKSGAMLGAGVLIVGQGTIGQNVCLGSCTTVINTSIKSGTTIEAGSLVGDTSRQFPEKESASSQGIKEDNNGFSDDRHLTANTQNKESQTNKNSSNKPEFVQEMEDLWADPEPEIEEVTEIPEIPTKPNAPADNNNAPVVGQVYINQLLCTLFPDRQAFNQSQNHSASDNSANNNK

SEQ ID NO: 59 - protein, 220 aa, Artificial Sequence
  Microcystis_aeruginosa_641538803/1-220
MSLPPVQPISRSEFYVNGDVTIDESAIVAPGVILRAAPNSQIIIGAGACLGMGTILTAYQGVIAIGAGAILGTGVLVVGRGEIGENACIGSTTTIFNASVAAMSLVPSGSLIGDTSRQITIEVSATRSEPERPPLPEPEPVVSQVSPVPSVEEVVAETVASPWDSEEMVAEASPAETREQASTTNRPNQASVVGKVYINQLLVTLFPERHRFNGNNNHNS

SEQ ID NO: 60 - protein, 265 aa, Artificial Sequence
  Nodularia_spumigena_640024190/1-265
MSVPPLHLSNNFDSYTSGEVTIHPSAVLAPGVILQAAVNSKMIIGPGVCIGMGSILQVSEGTLEVEAGANLGAGFLMVGKGKIGANACVGSATTVFNCSIEPGKVIPPGSILGDTSRQIEDTEQLESSTNNGDHTSTEQQPEAENSLETDEETVISSTTISAKAYWKFKHQSTSSSGSSPTSSSQPAPVEPAPVEPAPVEPAPVEQKAKASNSIPQKSKSSQPPTESPNSFGNQIYGQVSINRLLVTLFPHRQTLNDSISDDQSE

SEQ ID NO: 61 - protein, 248 aa, Artificial Sequence
  Nostoc_sp._PCC7120_637231228/1-248
MSVPPLRLHNNFDSYISGEVTIHPSAVIAPGVILQAAANSKIIIGAGVCIGMGSILQVDEGTIEVEAGASLGAGFLMVGQGKIGINACIGAATTLFNSSIPPALVVPPGSILGDTTRQVAATQSPSTSKNQVGETTQKPKENESKVITSTTLSASAFVEFKQHSVSVTEPPPSSENQSATVEENTTNGTDPNVTELSPEDSASDQPATESPNSFGTQIYGQGSIQRLLVTLFPHRQALNNPVSDDSSE

SEQ ID NO: 62 - protein, 248 aa, Artificial Sequence
  Anabaena_variabilis_646569975/1-248
MSVPPLRLHNNFDSYISGEVTIHPSAVIAPGVILQAAANSKIIIGAGVCIGMGSILQVDEGTIEVEAGASLGAGFLMVGQGKIGTNACIGAATTLFNSSIPPALVVPPGSILGDTTRQLAATESPATSTNQVDEATQKPKENESKVITSTTLSASAFVEFKQHSVSVTEPPPSPENQSATVEENTTNGTDPNVTELSPEDSASDQSATESPNSFGTQIYGQGSIQRLLVTLFPHRQALNNPVSDDSSE

SEQ ID NO: 63 - protein, 257 aa, Artificial Sequence
  Nostoc_punctiforme_642603263/1-257
MSVLSLRLSNNFDSYISGEVTIHPSAVLAPGVILQAAENSKIVIGPGVCIGMGAILQVHEGTLEVEAGANLGAGFLMVGKGKIGANACIGSATTVFNYSVEPGQVVPPGSILGDTSRQIAQTTQPEPSTNNSTATSVPPQKEEENGSGGVKEKVSSSTNFSAAAFVDFKQNKSISYFKSPATPESQPPPLEEPAKDAESPLQEAVQEPTKSDSDPNQLPTESPNGFGTQIYGQGSISRLLTTLFPHRQSLSDPNSDD

SEQ ID NO: 64 - protein, 235 aa, Artificial Sequence
  Synechococcus_sp_PCC7002_641611809/1-235
MTFQAITHPDIQISGDVRIHPRAVIAPGVILQATEGNYVAIATGACIGAGAIIQAHGGNIEIHAGAIIGAGCLIIGQCSVGENACLGYGSTLFQAAIAAAAILPPQSLIGDPSRQETTASYQTQPPKPANQSTTQPLDPWQAEDTTNQTATTFSPPGRSPTSSSNRPNVQPPPEAGSPPTETPNTEVMPTVPESKESLESGEKTPVVGQVYINQLLMTLFPHQNSLNTPNQPDEP

SEQ ID NO: 65 - protein, 241 aa, Artificial Sequence
  Synechocystis_sp._PCC_6803_637009624/1-241
MQLPPVHSVSLSEYFVSGNVIIHETAVIAPGVILEAAPDCQITIEAGVCIGLGSVISAHAGDVKIQEQTAIAPGCLVIGPVTIGATACLGSRSTVFQQDIDAQVLIPPGSLLMNRVADVQTVGASSPTTDSVTEKKSPSTANPIAPIPSPWDNEPPAKGTDSPSDQAKESIARQSRPSTAEAAEQISSNRSPGESTPTAPTVVTTAPLVSEEVQEKPPVVGQVYINQLLLTLFPERRYFSS

SEQ ID NO: 66 - protein, 229 aa, Artificial Sequence
  Synechococcus_sp._JA-3-3Ab_637873164/1-229
MPLPTSTTLRSWPSQNGETRYYVSGEVQVEAGAGIAAGVLLRANPGCRIEIGRGVCIGMGSILHACGGSLVVEAGATLGMGVLVIGQGTIGKNACIGSETTLLNCSVLSQAVIPPRSLVGDPTYPSRQEAEVGMASEAEPVSAAAPQEPIEPPEETLPEPTPPSPPDSPLAQVEKQTRRWQEAAEQTQENSRSPKTRKLNGIPGYSELDRLLGKIYPYRQILSSGGGQS

SEQ ID NO: 67 - protein, 219 aa, Artificial Sequence
  Synechococcus_sp._JA-2-3B′a(2-13)_637876191/ 1-219
MTLRALPGQNDETRYFVSGEVQVEAGAGIGAGVLLRANPGCRIHIGRGACIGMGSVLHACGGSLIVEAGATLGMGVLVIGQGTIGKNACIGSETTVLNCSVLSQAVIPPGSLIGDPTYGFDLQEAGGSKPIPAEPSPAAVEMAPEMSPEPSPPPSSPVANVEKQTRRWQEAAEQTQEKSGSPRTKTRNLNGIPGHWELDRLLSKIYPHRQVLSSGDSRL

SEQ ID NO: 68 - protein, 186 aa, Artificial Sequence
  Acaryochloris_marina_MBIC11017_641254454/1-186
MQLSPPQPVSTSQFCVIGDVTIHPHAKIAPGVILQAAPQSKIVIGASACIGIGAVIQAFDGTITVESNAVLGAGVLVLGKATIGVNACIGDCTTIINTDIVTQQVIPEGSLMGDASRSTIDESPNRSPFDDSLPSTPVNTAWPSSPPPIPNPTPASPPQRQSHVIGRAYVTQMLQVLFARNSSPYP

SEQ ID NO: 69 - protein, 244 aa, Artificial Sequence
  Cyanothece_sp_PCC8801_643474672/1-244
MYLPLIRPATHSDICVIGDVTIHDNAVIAPGTILQAAPGCRILIKEGACIGMGSLLNAYNGDIEVASGAMLGAGVLVVGHSQIGQNACIGSSTTIINSSIDSGTAIAPGSLLGDQSRQVTAETSEPTKELKSENNGSVTNNNSSISNKNNIFSKVQPTEDKKPNFVEEMQDLWAEPEPEVEPIAEVSPPPKPSVDPIPEVVAEPKPSPEPQNAPVVGQIYINQLLYTLFPERQAFNRSQNGSSS

SEQ ID NO: 70 - protein, 214 aa, Artificial Sequence
  Desulfatibacillum_alkenivorans_643538193/1-214
MKLTEEMLRQIITEVVGQMAGGAAAPAPAAVDTDKPLNFIEKGPAQAGSNPKEVVVAVPPGFGVTPTKTIIDIPHSVVLAEVAAGIEEEGLTARFVRNYQTADVAFLAHSAAQLSGSGVGIGILSRGTSVIHQKDLAPLQNLELFPQAPLVEAETFRAIGKNAAKYAKGENPNPVPVKNDPMARPRYQGLAALLHNKEVQFLDPQKKILEVVQG

SEQ ID NO: 71 - protein, 223 aa, Artificial Sequence
  Dethiosulfovibrio_peptidivorans_2501566254/ 1-223
MINEELVRKVIAEVLQEVAASENVESASVTARPSAPAVKAEISMEMTEKERATRGTDAREVVVAIPPAFGTEFDATIVDVSLADVLRQVFAGIEEQGLSWRLVRVYHTADVAFIAHQAAKLSGSGVGIGIISRGTTVIHQRDLAPLNNLELFPQSPLLDLETFRAIGRNAGMYAKGEQPVPVATKNDPMARPKFQGIAALLHNKEVKALDRSKSPMELQVRFR

SEQ ID NO: 72 - protein, 239 aa, Artificial Sequence
  Lactobacillus_brevis_639653783/1-239
MAQEIDENLLRNIIRDVIAETQTGDTPISFKADAPAASSATTATAAPVNGDGPEPEKPVDWFKHVGVAKPGYSRDEVVIAVAPAFAEVMDHNLTGISHKEILRQMVAGIEEEGLKARIVKVYRTSDVSFCGAEGDHLSGSGIAIAIQSKGTTIIHQKDQEPLSNLELFPQAPVLDGDTYRAIGKNAAEYAKGMSPSPVPTVNDQMARVQYQALSALMHIKETKQVVMGKPAEQIEVNFN

SEQ ID NO: 73 - protein, 229 aa, Artificial Sequence
  Thermoanaerobacter_sp._X514_641542302/1-229
MVKTESLVEQIVKEVLKKLENVEIAAPATQSSDDANQEWEMIIEEIGEAKQGVNVDEVVIGVSPGFYIKFKKNIIGIPLGNILREIISGITEQGLKARIVRVKHTADVGFIAHTAAKLSGSGIGIGIQSRGTVVIHQKDLQPLNNLELFPQCPVLTLETYRAIGRNAALYAKGESPTPVPVQNDQMARPKYQAIAAVMHNFETKYVQTGAKPVELKVSFARKGGNKSDR

SEQ ID NO: 74 - protein, 224 aa, Artificial Sequence
  Thermosediminibacter_oceani_2503264369/1-224
MINTEMVVEEVVKEVLKRLAGEREKVAEDYAVGNPAGKELLLEEMGEAKPGAREEEVVIGVSPAFGVKFKENINGIPLADILREIMAGIAEEGLNSRVIRVRHTADVAFIGHTAAKLSGSGVGIGIQSRGTAVIHHKDLQPLNNLELFPQCPVMTLDTYRAIGKNAALYAKGESPTPVPVMNDQMARPKFQAKAAVMHNFETQYVKPGLKPVELKVCFSKGGTS

SEQ ID NO: 75 - protein, 221 aa, Artificial Sequence
  Yersinia_bercovieri_638773784/1-221
MVDINEKLLRQIIEGVLQEMQGEKNSVSFKQESQPATAVASGDFLTEVGEARPGSNQDEVIIAVGPAFGLSQTANIVGIPHKNILRELIAGIEEEGIKARVIRCFKSSDVAFVAVEGNRLSGSGISIGIQSKGTTVIHQQGLPPLSNLELFPQAPLLTLETYRLIGKNAARYAKRESPQPVPTLNDQMARPKYQAKSAILHIKETKYVVTGKNPQELRVAL

SEQ ID NO: 76 - protein, 222 aa, Artificial Sequence
  Shigella_sonnei_640429818/1-222
MEINEKLLRQIIEDVLAEMQPSDKSVSFRAPVSATVPSAPDTGNFLTEIGEAQQGTQQDEVIIAVGPAFGLAQTVNIIGIPHKNILREVIAGIEEEGIKARVIRCFKSSDVAFVAVEGDRLSGSGIAIGIQSKGTTVIHQQGLPPLSNLELFPQAPLLTLDTYRQIGKNAARYAKRESPQPVPTLNDQMARPKYQAKSAILHIKETKYVVTGKKPQELRVTF

SEQ ID NO: 77 - protein, 222 aa, Artificial Sequence
  Escherichia_coli_E24377A_640925948/1-222
MEINEKLLRQIIEDVLAEMQPSDKSVSFRAPVSATVSSAPDTGNFLTEIGEAQQGTQQDEVIIAVGPAFGLAQTVNIIGIPHKNILREVIAGIEEEGIKARVIRCFKSSDVAFVAVEGDRLSGSGIAIGIQSKGTTVIHQQGLPPLSNLELFPQAPLLTLDTYRQIGKNAARYAKRESPQPVPTLNDQMARPKYQAKSAILHIKETKYVVTGKKPQELRVTF

SEQ ID NO: 78 - protein, 229 aa, Artificial Sequence
  Klebsiella_pneumoniae_647940093/1-229
MEINETLLRQIIEEVLSEMKSGADKPVSFSAPAASVASAAPVAVAPVSGDSFLTEIGEAKPGTQQDEVIIAVGPAFGLAQTANIVGIPHKNILREVIAGIEEEGIKARVIRCFKSSDVAFVAVEGNRLSGSGISIGIQSKGTTVIHQRGLPPLSNLELFPQAPLLTLETYRQIGKNAARYAKRESPQPVPTLNDQMARPKYQAKSAILHIKETKYVVTGKNPQELRVAL

SEQ ID NO: 79 - protein, 224 aa, Artificial Sequence
  Salmonella_enterica_enterica_sv_Typhi_Ty2_ 637404647/1-224
MEINEKLLRQIIEDVLRDMKGSDKPVSFNTPAASTAPQTAAPAGDGFLTEVGEARQGTQQDEVIIAVGPAFGLAQTVNIVGLPHKSILREVIAGIEEEGIRARVIRCFKSSDVAFVAVEGNRLSGSGISIGIQSKDTTVIHQQGLPPLSNLELFPQAPLLTLETYRQIGKNAARYAKRESPQPVPTLNDQMARPKYQAKSAILHIKETKYVVTGKNPQELRVTL

SEQ ID NO: 80 - protein, 224 aa, Artificial Sequence
  Salmonella_typhimurium_LT2_637212760/1-224
MEINEKLLRQIIEDVLRDMKGSDKPVSFNAPAASTAPQTAAPAGDGFLTEVGEARQGTQQDEVIIAVGPAFGLAQTVNIVGLPHKSILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 81 - protein, 224 aa, Artificial Sequence
  Citrobacter_koseri_640914761/1-224
MEINEKLLRQIIEDVLSEMQTSDKPVSFRAPTASTSPQAAAPQDDGFLTEIGEARQGTQQDEVIIAVGPAFGLSQTVNIVGLPHKNILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 82 - protein, 224 aa, Artificial Sequence
  Sebaldella_termitidis_646428071/1-224
MNIDEKQLKDIIAGVIKEIQNEKGNCGCTSDGKISFGQGSSDNRLKLNENGQAKQGTRSDEVVIGIAPAFGESQTETIMHVPLYKVLREIIAGIEEEGLKFRIIRVTRTSDVCFIAHDAAKLSGSKIGIGIQSKGTAVIHQADLMPLSNLELFPQCPLLDLETYRAIGKNAAKYAKGETPNPVPVRNDQMVRPKYQALAAILHIKETEHVIPLSKPVELEAIFS

SEQ ID NO: 83 - protein, 175 aa, Artificial Sequence
  Lactobacillus_brevis_639653782/1-175
MSEIDDLVAKIVQQIGGTEAADQTTATPTSTATQTQHAALSKQDYPLYSKHPELVHSPSGKALNDITLDNVLNDDIKANDLRITPDTLRMQGEVANDAGRDAVQRNFQRASELTSIPDDRLLEMYNALRPYRSTKAELLAISAELKDKYHAPVNAGWFAEAADYYESRKKLKGDN

SEQ ID NO: 84 - protein, 179 aa, Artificial Sequence
  Dethiosulfovibrio_peptidovorans_2501566255/ 1-179
MEINEKLIAEMVRQVLQSGGNQEKGASNSPQETSVKDRKVLSKNDYPLAVKRPELLVGPRGKGFDELTLSNIESGNVAFEDFKITPDALEYQAQIAEDDGCHQIAVNLRRAAELTKVPDSRVLEIYNAMRPHRSTKSDLLGIADELEKNYGAMVCAELLRETADVYERRKLLKGDLPTG

SEQ ID NO: 85 - protein, 166 aa, Artificial Sequence
  Sebaldella_termitidis_646428072/1-166
MDEVMIKNMVKEILNNIEKHDSGKKDSSGKIGVSSYPLGSRRPDLVRTPTNKTLDDITLENVMNGKITIEDLNITADTLELQAQVAEDAGRSSIARNFRRAAELTTIPDDRILQIYNSLRPFRSTKAELLQIADELENKYGALINAALVREAAEVYEKRKKLRSDD

SEQ ID NO: 86 - protein, 174 aa, Artificial Sequence
  Yersinia_bercovieri_638773783/1-174
MNSEAIESMVRDVLNKMNSLQGQAPAACPAPAASSRSDAKVSDYPLANKHPDWVKTATNKTLDDLTLANVLNGSVTSQDLRITPEILRIQASIAKDAGRPLLAMNFERAAELTAVPDDKVLDIYNALRPFRSSKEELNAIADDLEKTYQATICAAFVREAAVLYVQRKKLKGDD

SEQ ID NO: 87 - protein, 172 aa, Artificial Sequence
  Shigella_sonnei_640429819/1-172
MNTDAIESMVRDVLNRMNSLQDAAPVSAVPNASILSAKVTDYPLANKHPEWVKTATNKTLDDFTLENVLSDNVTALDMRITPETLRIQAAIARDAGRDRLAMNFERAAELTSVPDDRILEIYNALRPYRSTKQELIAIADDLEQRYQAKICAAFVREAAELYVERKKLKGDD

SEQ ID NO: 88 - protein, 172 aa, Artificial Sequence
  Escherichia_coli_E24377A_640925949/1-172
MNTDAIESMVRDVLNRMNSLQDAAPVSAVPNASILSAKVTDYPLANKHPEWVKTATNKTLDDFTLENVLSDNVTALDMRITPETLRIQAAIARDAGCDRLAMNFERAAELTSVPDDRILEIYNALRPYRSTKQELIAIADDLEQRYQAKICAAFVREAAELYVERKKLKGDD

SEQ ID NO: 89 - protein, 173 aa, Artificial Sequence
  Salmonella_enterica_637404646/1-173
MNTDAIESMVRDVLSRMNSLQGDAPAAAPAAGGTSRSAKVSDYPLANKHPEWVKTATNKTLDDFTLENVLSNKVTAQDMRITPKTLRLQASIAKDAGRDRLAMNFERAAELTAVPDDRILEIYNALRPYRSTKEELLAIADDLENRYQAKICAAFVREAAGLYVERKKLKGDD

SEQ ID NO: 90 - protein, 173 aa, Artificial Sequence
  Salmonella_typhimurium_LT2_637212761/1-173
MNTDAIESMVRDVLSRMNSLQGDAPAAAPAAGGTSRSAKVSDYPLANKHPEWVKTATNKTLDDFTLENVLSNKVTAQDMRITPETLRLQASIAKDAGRDRLAMNFERAAELTAVPDDRILEIYNALRPYRSTKEELLAIADDLENRYQAKICAAFVREAAGLYVERKKLKGDD

SEQ ID NO: 91 - protein, 172 aa, Artificial Sequence
  Citrobacter_koseri_640914760/1-172
MNTDAIESMVRDVLSRMNSLQGNAPAPAAASASTHTAKVTDYPLANKHPEWVKTATNKTLDEFTLENVLSDKVTAQDMRITPDTLRIQAAIARDAGRDRLAMNFERAAELTAVPDDRILEIYNALRPYRSTKEELMAIADDLENRYQAKICAAFVREAATLYVERKKLKGDD

SEQ ID NO: 92 - protein, 174 aa, Artificial Sequence
  Klebsiella_pneumoniae_640800248/1-174
MNTDAIESMVRDVLSRMNSLQDGITPAPAAPTNDTVRQPKVSDYPLATRHPEWVKTATNKTLDDLTLENVLSDRVTAQDMRITPETLRMQAAIAQDAGRDRLAMNFERAAELTAVPDDRILEIYNALRPYRSTQAELLAIADDLEHRYQARLCAAFVREAAGLYIERKKLKGDD

SEQ ID NO: 93 - protein, 174 aa, Artificial Sequence
  Thermoanaerobacter_sp._X514_641542301/1-174
MIDEKTLEIIVREVLTNLTSDKGTQNQQKTASSSLPKLDPKRDYPLAKNKPELAKSITGKTINEITLQAVREGKVLPDDLKISPETLLAQAEIAEAAGRKQLANNFRRAAELTKVPDKRILEIYNALRPYRSTKEELLAIADELDNAYGAKVCAAFVREAAEVYERRGRLKGME

SEQ ID NO: 94 - protein, 171 aa, Artificial Sequence
  Thermosediminibacter_oceani_2503264370/1-171
MIDEKALEEIVRQVLEELGSHKKQVKAEIKKDEGLDPKLDFPLSKKRPELLKSATGKKFTEITFEEALRGNVRAEDFRISPDTLLIQAEIAERVGRKQFANNLRRAAELTRVPDERILEIYNALRPYRSTKEELLAIADELQQKYDAPICAAFVREAAEVYERRRRLKGME

SEQ ID NO: 95 - protein, 306 aa, Artificial Sequence
  Streptococcus_sanguinis_640103604
MDELQLKEMIRSLLNEMGGDSAVKETAATDQNKAEKPAVSLQEEVKQDTSVIEDGIIPDITEVDIQEQFLVPNAINEEAYRKIKKFTPARLGLWRAGDRYKTQSVLRFRADHAAAQDAVFSYVSDDFIKEMGFIPVQTKATTKDEYLTRPDFGRVFPEDQQAIIKEKCKPNAKVQIVVGDGLSSSAIEANVKDFLPALKQGLKMFGLDFGEVLFIKHARVAAMDQIAELTGAEVICMLVGERPGLVTAESMSAYLAYKPTVGMPEAKRTVVSNIHKGGTPAVEAGAYVAEIIKKILDNKKSGIDLK

SEQ ID NO: 96 - protein, 300 aa, Artificial Sequence
  Photobacterium_profundum_3TCK_639100602
MNEQKIQDIVATVLAQLGETNVAASDITKVVNAVTPAAGGYVPQVSAESLPDLGDIQFKKWNGIQNAVDKKVVEDLMSQTDARVGTGRTGPRPRTTALLRFLADHSRSKDTVIKNVESSWLQERNLMEVQSCASDKDEYLTRPDLGRKLNDAGKALIQSNCKKAPQVQVVLSDGLSLDAVTVNHDEILPPLLNGLKNAGLDVGTPFFLRYGRVKAQDEIGMLLNAEVNLLLIGERPGLGQSESLSCYAIYKPTSETVESDRTVISNIHAGGTPPVEAAAVIVDLVKNMLEKKASGIKLKR

SEQ ID NO: 97 - protein, 339 aa, Artificial Sequence
  Bacillus_sp_B14905_640620702/1-339
MSRVNDQLVSMITQLVMEKMEKTTEGQAPEVITTRTEEPLIKFYDTAATKGATELAKPMSTTSEPLIQLYQQGTPQQAHIAPATFEQPLNVAVPIKPFQFEADTLTDSIQAAKKHTPARIGVGRAGTRPKTKTWLKFRLDHAAAVDAVYGEVTEYLLQKLDVFQVTTKVTDKEEYITRPDLGRRLSDEAKSLIQQKCKQQPKVQIIISNGLSASAIEENVQDVYLALQQSLSNLNIDIGTTFYIDKGRVALMDEIGELLQAEVIVYLIGERPGLVSAESMSAYLCYKPRIGTVEAERMVISNIHKGGIPPLEAGAYLGTIVQKILHYEASGVELVAKEG

SEQ ID NO: 98 - protein, 340 aa, Artificial Sequence
  Nocardioides_sp_JS614_639778639/1-340
MSTDELRSIVAEVLAELAEPGDAFARLTTPATTAGPSGPTSTPAPEESDAPSSAATEPAAVPASSATEITRPTLSGAPVSIEVSDPTVPEARHRIGVENPANPSGLANLAASTAARIAVGRAGPRPRTESVLLFGADHAVTQDAIFGDVPTALLDQFGLFAVQTKVTTQDEFLLRPDLGRELDDAAKLVVAEKCVKGPQVQIVVGDGLSAAAVTNNLPQIYPVLEAGLRDAGLTLGTPFFVRYCRVGVINDINDIVGADVVVLLIGERPGLGVADALSVYSGWRPTAGKTDAHRDVICMITQNGGTNPLEAGAFAVEHVKNVMKHQASGVELRLQESGTR

SEQ ID NO: 99 - protein, 316 aa, Artificial Sequence
  Marinobacter_aqueolei_639811210/1-316
MDEQTIQSIVNSVLRELGEKDLPAGQVTRVQPEGKSTQRNDPPAYKPSETAGRQGQTESADTGDGLEDLSLEKFVHWNGIENAHNASVNSDMVKQTAARVCQGRAGPRPRTRSLLRFLADHSRSKDTVVKEVSPEWLEKKNLWEVQTCISDKSEYLRRPDLGRKLSDDAKKTIGERCKKSPQVQVVISDGLSTDAVTNNLDEIIPPLMKGLESAGFTVGTPFFLRYGRVKAQDEIGNLLQADANLLLIGERPGLGQSESLSCYCVYKPTEKTVESDRMVISNIHKGGTPPIEAAAVIVDLTRKMLEQKASGLNLKR

SEQ ID NO: 100 - protein, 298 aa, Artificial Sequence
  Shewanella_benthica_KT99_641463123/1-298
MNEQNIKNIVATVLAQLGENNIQPSTITKVIDAASNVAGKTVISDESLPDLGEPRFKKWNGVINAANPSIVDDLMSQTNARMGTGRTGPRPRTIPLLRFLADHSRSKDTVIKNVESSWLQERGLMEVQSAAKDKDEYLTRPDLGRKLNDEAIVLIKEKCKQAPQVQVILSDGLSLDAVTANHDEILPALLNGLKSAGLDVGTPFFLRFGRVKAQDEIGMLLNADVNILLIGERPGLGQSESLSCYAVYKPSEDTVESDRTVISNIHAGGTPPVEAAAVIVDLVKDMLKQKTSGINLKR

SEQ ID NO: 101 - protein, 291 aa, Artificial Sequence
  Yersinia_intermedia_638787901/1-291
MDQKQIEEIVRSVMLRMGQVEVATQPASAAASADTVECCSMDLGSEEAKQWIGVTNPQRLDVLQELRSSTAARVCTGRAGPRPRTQALLRFLADHSRSKDTVLKEVPLEWVQKHGLLEVQSEISDKNLYLTRPDMGRCLSASAIETLKTQCKANPDVQVVISDGLSTDAITANYDEILPPLLKGLELAGMNVGTPFFVRYGRVKIEDQIGELLGAKVVILLVGERPGLGQSESLSCYAVYSPRVATTVEADRTCISNIHRGGTPPVEAAAVIVDLAKRMLEQKASGISMTR

SEQ ID NO: 102 - protein, 299 aa, Artificial Sequence
  Klebsiella_pneumoniae_640799824/1-299
MDQKQIEDIVRSVMASMGQPQSQPQAPAASTPACHAACASEAVVESCALDLGSAEAKAWIGVQHPHRAEVLTELKRSTAARVCTGRAGPRPRTQALLRFLADHSRSKDTVLKEVPEAWVKAQGLLEVRSEISDKNLYLTRPDMGRRLSPEAIDALKAQCVMDPDVQVVVSDGLSTDAITANYEEILPPLLAGLKQAGLKVGTPFFVRYGRVKIEDQIGEILGAKVVILLVGERPGLGQSESLSCYAVYSPRVATTVEADRTCISNIHQGGTPPVEAAAVIVDLAKRMLEQKASGINMSR

SEQ ID NO: 103 - protein, 298 aa, Artificial Sequence
  Salmonella_enterica_paratyphi_637600699/1-298
MDQKQIEEIVRSVMASMGQDVPQPVAPSKQEGAKPQCASPTVTESCALDLGSAEAKAWIGVENPHRADVLTELRRSTAARVCTGRAGPRPRTQALLRFLADHSRSKDTVLKEVPEEWVKAQGLLEVRSEISDKNLYLTRPDMGRRLSPEAIDALKSQCVMNPDVQVVVSDGLSTDAITANYEEILPPLLAGLKQAGLNVGTPFFVRYGRVKIEDQIGEILGAKVVILLVGERPGLGQSESLSCYAVYSPRVATTVEADRTCISNIHQGGTPPVEAAAVIVDLAKRMLEQKASGINMTR

SEQ ID NO: 104 - protein, 298 aa, Artificial Sequence
  Salmonella_typhimurium_LT2_637213175/1-298
MDQKQIEEIVRSVMASMGQDVPQPAAPSTQEGAKPQCAAPTVTESCALDLGSAEAKAWIGVENPHRADVLTELRRSTAARVCTGRAGPRPRTQALLRFLADHSRSKDTVLKEVPEEWVKAQGLLEVRSEISDKNLYLTRPDMGRRLSPEAIDALKSQCVMNPDVQVVVSDGLSTDAITANYEEILPPLLAGLKQAGLNVGTPFFVRYGRVKIEDQIGEILGAKVVILLVGERPGLGQSESLSCYAVYSPRVATTVEADRTCISNIHQGGTPPVEAAAVIVDLAKRMLEQKASGINMTR

SEQ ID NO: 105 - protein, 301 aa, Artificial Sequence
  Citrobacter_koseri_640914312/1-301
MDQKQIEEIVRSVMASMGESQPQAPAESAPACSAKQCAAPSAPSAAESCALDLGSAEAKAWVGVENPHRADVLAELRRSTAARVCTGRAGPRPRTLALLRFLADHSRSKDTVLKEVPEEWVKAQGLLEVRSEISDKNLYLTRPDMGRRLSQEAIDALKAQCVASPDVQVVISDGLSTDAITANYEEILPPLLSGLKQAGLKVGTPFFVRYGRVKIEDQIGEILGAKVVILLVGERPGLGQSESLSCYAVYSPRVATTVEADRTCISNIHQGGTPPVEAAAVIVDLAKRMLEQKASGINMTR

SEQ ID NO: 106 - protein, 295 aa, Artificial Sequence
  E_coli_HS_640921698/1-295
MDQKQIEEIVRSVMASMGQTAPAPSEAKCATTNCAAPVTSESCALDLGSAEAKAWIGVENPHRADVLTELRRSTVARVCTGRAGPRPRTQALLRFLADHSRSKDTVLKEVPEEWVKAQGLLEVRSEISDKNLYLTRPDMGRRLCAEAVEALKAQCVANPDVQVVISDGLSTDAITVNYEEILPPLMAGLKQAGLKVGTPFFVRYGRVKIEDQIGEILGAKVVILLVGERPGLGQSESLSCYAVYSPRMATTVEADRTCISNIHQGGTPPVEAAAVIVDLAKRMLEQKASGINMTR

SEQ ID NO: 107 - protein, 303 aa, Artificial Sequence
  Alkaliphilus_oremlandii_641246983/1-303
MDELNLKEMIKSILNEMVGEAPPAVINSNSTAERSVGTMQTTKPQGVEERFIPDITAVDIRKQFLVPNAADKEGYLKMKSYTPARLGLWRAGPRYMTEPSLRFRADHAAAQDAVFSYVDEDLVKELGFVEVVTECKDKDEYLTRPDLGRKFSNEAINTIKKVVKPNQKVQVIVGDGLSSAAIEANIKDVLPSLRQGLKMFGLDFGEVVFIKHCRVPAMDPIGEATGAEVVCLLIGERPGLVTAESMSAYIAYKPTIGMPEARRTVVSNIHRQGTPAVEAGAYIAEIIKRMLDNKASGLDLKEK

SEQ ID NO: 108 - protein, 303 aa, Artificial Sequence
  Enterococcus_faecalis_647309386/1-303
MNEKELKEMIAGILTEMVADNQAVSTATVTAEEKPVTTHVTETTEIEEGLIPDITEVDLRKQLLLKNAVDPEALLKMKAFSPARLGVGRAGTRYMTSSTLRFRADHAAAQDAVFSDVSEDLVKEMNFISTKTICNSKDEYLTRPDYGRQFDEENSEIIRKNTTPKAKIQMVVGDGLSSAAIEANIKEVLPAIKQGLNMYNLDFDNVVFVKYCRVPAMDKIGEITDADVVCLLVGERPGLVTAESMSAYIAYKPTVGMPEARRTVISNIHKGGTPAVEAGAYIAEIIKKMLDKKKSGIDLKEAE

SEQ ID NO: 109 - protein, 293 aa, Artificial Sequence
  Listeria_monocytogenes_10403S_646521862/1-293
MNEQELKQMIEGILTEMSGGKTTDTVAAVPTKSVVETVVTEGSIPDITEVDIKKQLLVPEPADREGYLKMKQMTPARLGLWRAGPRYKTETILRFRADHAVAQDSVFSYVSEDLVKEMNFIPVNTKCQDKDEYLTRPDLGREFDDEMVEVIRANTTKNAKLQIVVGDGLSSAAIEANIKDILPSIKQGLKMYNLDFDNIIFVKHCRVPSMDKIGEITGADVVCLLVGERPGLVTAESMSAYIAYKPTVGMPEARRTVISNIHSGGTPPVEAGAYIAELIHNMLEKKCSGIDLK

SEQ ID NO: 110 - protein, 296 aa, Artificial Sequence
  Clostridium_phytofermentans_641293737/1-296
MDEQSLRKMVEQMVEQMVGGGTNVKSTTSTSSVGQGSATAISSECLPDITKIDIKSWFLLDHAKNKEEYLHMKSKTPARLGVGRAGARYKTMTMLRVRADHAAAQDAVFSDVSEEFIKKNKFVFVKTLCKDKDEYLTRPDLGRRFGKEELEVIKKTCGQSPKVLIIVGDGLSSSAIEANVEDMIPAIKQGLSMFQINVPPILFIKYARVGAMDDIGQATDADVICMLVGERPGLVTAESMSAYICYKAKHGVPESKRTVISNIHRGGTTPVEAGAHAAELIKKMLDKKASGIELKG

SEQ ID NO: 111 - protein, 293 aa, Artificial Sequence
  Clostridium_difficile_630_640157742/1-293
MNEKDLKALVEQLVGQMVGELDTNVVSETVKKATEVVVDNNACIDDITEVDIRKQLLVKNPKDAEAYLDMKAKTPARLGIGRAGTRYKTETVLRFRADHAAAQDAVFSYVDEEFIKENNMFAVETLCKDKDEYLTRPDLGRKFSPETINNIKSKFGTNQKVLILVGDGLSSAAIEANLKDCVPAIKQGLKMYGIDSSEILFVKHCRVGAMDHLGEELGCEVICMLVGERPGLVTAESMSAYIAYKPYIGMAEAKRTVISNIHKGGTTAVEAGAHIAELIKTMLDKKASGIDLK

SEQ ID NO: 112 - protein, 299 aa, Artificial Sequence
  Alkaliphilus_metalliredigens_QYMF_640781165/ 1-299
MISEQAVKEMVQQIVEQMTIGQKQTTEDKYTQETDGKEQPEICIEDKNLKDLTEIKMQDYFAVPNPENKEVYLGLKEQTPARVGIWRTGSRNSTETLLRFRADHAVAMDAVFTYVSEELLEEVGLFSVNTLCRNKDEYMTRPDLGRKFSQETIEMIKEKCVKSPQVQIYVSDGLSSTAIEANIKDILPSIMQGLENEGLKVGTPFFVKHGRVPAMDVISETLDAGATVVLIGERPGLATGESMSCYMTYGGTVGMPESRRTVISNIHRGGTPATEAGAHIAQIVKEMINQKASGLDLKL

SEQ ID NO: 113 - protein, 297 aa, Artificial Sequence
  Thermanaerovibrio_acidaminovorans_646433235/ 1-297
MVKEQDLKQLVMEILNEMSRGAEPSPTQPSTPPQGAQEAPSGQEGELPDLTQVDIRTQCLVPSPKDPAALMAMKAKTPARIGVWRAGPRYKTETLLRFRADHAAAQDAVFSEVSDEFLAKNDLQVVKTECADKDQFLTRPDLGRRFSPEATETIKRLVGSPPKVLVYISDGLSTTAVETNAIDTFKAMAQALDRQGIKLPKPFFVKYGRVPAMDVISQVTGAEVVCVLIGERPGLVTAESMSAYITYKGTVGMPEAKRTVVSNIHSGGTPAVEAGGYVAEIIKLMLEKRASGIDLKL

SEQ ID NO: 114 - protein, 303 aa, Artificial Sequence
  Bacteroides_capillosus_641047988/1-303
MREVHAMNEKDLRSIIEQVLAEMNGAGEAKEAAPSCCTAAPVEESCKVEEGCLPDITEIDIREQYLVKDPENGEEYAELKMNAPCRLGIGKAGARYNTLPQLEFRAAHSAAQDAVFNDVDAEFVEKMGLWTVQTQCDSKDTYLTRPDLGRKLSPEAVETIKAKCKKNPTVQIYVADGLSSAAVAANIGDLLPALMQGLQSYKIDVGTPFFVKYGRVGVMDEISELTGAEVTCTLIGERPGLITAESMSAYIAYKATVGMPEARRTVVSNIHRAGTIPAEAGAHIAEIIKIMLEKKASGTDLKL

SEQ ID NO: 115 - protein, 295 aa, Artificial Sequence
  Fusobacterium_nucleatum_647527653/1-295
MVSELELKEIIGKVLKEMAVEGKTEGQAVTETKKTSESHIEDGIIDDITKEDLREIVELKNATNKEEFLKYKRKTPARLGISRAGSRYTTHTMLRLRADHAAAQDAVLSSVNEDFLKANNLFIVKSRCEDKDQYITRPDLGRRLDEESVKTLKEKCVQNPTVQVFVADGLSSTAIEANIEDCLPALLNGLKSYGISVGTPFFAKLARVGLADDVSEVLGAEVTCVLIGERPGLATAESMSAYITYKGYVGIPEAKRTVVSNIHVKGTPAAEAGAHIAHIIKKVLDAKASGQDLKL

SEQ ID NO: 116 - protein, 299 aa, Artificial Sequence
  Sebaldella_termitidis_646428094/1-299
MLSERELREIIGKVIDEMGSNGKTDIPAAVGNDFKASSSVKENVSDDQLVDLGEINIKDQLLVDNPANREEYMKLKQRTSARLGIGRAGTRFKTDVLLRFRADHAAAQDAVFNDVPESFLEEAGLFEVTTECKDRDEYITRPDLGRKISAEGIKLLEEKCKKSPTVQVYVSDGLSSTAVEANTKNILPAVLNGLKGYGIDTGTPFFVKYGRVAAEDHISDILKPDVVCVLIGERPGLTTAESMSAYIVYKAYVGIPEAKRTVVSNIHKDGTPAAEAGAHVADLIKKILDAKASGQDLKL

SEQ ID NO: 117 - protein, 313 aa, Artificial Sequence
  Leptotrichia_buccalis_645005463/1-313
MLSERELKDVIEKIISEIKIEETPAKETPVTVMEEKTPVVSTSSTYDQDENPRENPHIVNGEVRDIGKINVKEQMLVDNPEDREEYMKLKQKTSARLGIGRAGTRMRTEVLLRLRADHAAAQDAVFNDVPTEFLDELGLFEITTECESRDQYITRPDLGRKISQEGIKIIEEKCKKNPTVQIVVSDGLSSTAIEANAKNIIPAMLNGLKGYGIDTGTPFFIKYGRVGAGDHVGEILNAEVVCILIGERPGLTTAESMSAYITYKARPGISEAKRTVVSNIHKDGTPSAEAGAHVATLIKKIIDAKASGQDLKL

SEQ ID NO: 118 - protein, 858 aa, Artificial Sequence
  n643125056_ANHYDRO_00930/1-858
MIERGFSKPTQRVERLRKVIINATPEVEADRARLITESYKETEGMSNILRRAKACEKLFKNLPVTIREDELVVGSLTKTPRSTGLCPEFSYSWVADEFDTMATRSADPFLIREETKEELKEIFKYWKGKTNSEYADSLMSQEAKDCIENGIFSVGNYFYGGVGHVTVDYGKILKRGFRGVLEEVILAMRKLDDKDPETIEKMQFYKALIITYTAAIKFAHRYSEKARELADKENDIKRKEELLKISDICKKVPEYGADTFWEACQSFWFIQLMVQIESNGHSISPGRFDQYMYPYLKNDSIDRELAQELVDCIWVKFNDINKTRDEVSAQAFAGYSMFQNLCVGGQDIHGLDATNDVSYMCMESVSHVALPAPSFSVRVHQNSPYEFLLRACEVSRLGYGVPAFYNDEVIILNLVSRGVKLEDARDYSIIGCVEPQASHKTEGWHDAAFFNAAKVLEITLNNGRCNGKQLGPVTGEITEMTSIEQIIEAFEKQMAYFVKYLAEADNCVDYAHMQRGNLPFMSALVDDCIKRGKSSQSGGALYNFTGPQAFGVADSGDSLYAIEKNVFENKRISLEELKEALENNFGFTDSIMPGPCGGDSVSAKVGQLSEAEIYDAIKKILSNSDTTDVDEIAKKLELNNTENSSYQSACGCSANETGRFKTIQKILDNTGSFGNDDQGCDEFAIRVAQIYCDEVDKYTNPRGGAFQAGIYPVSANVLFGKDVGALPDGRLAGAPLADGVSPRQGKDANGPTAAANSVAKLPHFQASNGTLYNQKFSPKSVEGEKGLKNFVSIIKSYFDHKGAHIQFNVIDRQTLIDAQENPQDHKDLLVRVAGYSAHFVTLAKDVQDDIISRTEHTM

SEQ ID NO: 119 - protein, 857 aa, Artificial Sequence
  n2501030921_PepasDRAFT_0461/1-857
MLEKGFSQPTERVKRLRQVIIDAVPQVESDRARLITESYKETEGLTNILRRAKAVEKLFNELPVTIRDDELIVGSITKAPRSTGLCPEFSYEWVEAEFDTMATRLADPFVIPEETKKELHEVFKYWKGKTTSEFADSLMSTEAKDCIANGIFTVGNYFYGGVGHVNVDYKKIIKKGFRGVLEETVKAMNEMDESEPEAIKKMQFYKAVIISYNAAINFAHRYAKKAEELANVETNPQRKQELLRIAENCKRVPEYGARDFWEACQAFWFVQIMVQIESNGHSISPGRFDQYMYPSYKADTTITKEFAQELVDCIWVKLNDLNKTRDEVSAQAFAGYAVFQNLCVGGQDSEGFDATNDVSYMCMEAVAHVALPAPSFSVRVHQNSPYEFLLRACEVSRLGYGVPAFYNDEVIVLNLVSRGVKIEDARDYSIIGCVEPQAGHRTEGWHDAAFFNIAKVLEITLNNGRCNGKQLGPKTGELTDMKSIDDIFVAYQKQMEHFVKYLAEADNCVDYAHMERGNLPFMSAMVDDCIKRGKSAQSGGAIYNFTGPQAFGVADSGDSLYAIYKNVFEDKKISLADLKEALEKNFGFTDSLMPGCGCNTQTVSAKVGEMNESEIYEAVKKILASTGSINVDDLENKLNEEYVVSGDCGCGSQETTGKFRTIQKILDNTDSFGNDNELCDEFAIRAAKIYCDEVDKYTNPRGGAFQAGIYPVSANVLFGKDVGALPDGRLAHAPLADGVSPRQGKDTTGPTAAANSVAKLPHGQASNGTLYNQKFSPQAVSGEKGLKNFVSIVRSYFDHKGAHVQFNVVDRNTLIEAQKNPQDHKDLLVRVAGYSAHFVTLAKEVQDDIINRTEHTM

SEQ ID NO: 120 - protein, 850 aa, Artificial Sequence
  n637358380_c4537/1-850
MLEKGFSNPTDRVVRLRNMILTAKPYVESERAVLATEAYKETEQLPAIMRRAKVVEKIFNQLPVTIRPDELIVGAVTINPRSTEICPEFSYDWVEKEFETMEHRIADPFVIPKKTAQELHEAFKYWPGKTTSALAASYMSEGTKESMASGVFTVGNYFFGGVGHVSVDYGKVLKIGFRGIINEVSRALESLDRTEPGYIKKEQFYNAVLISYNAAIRFAHRYAEEASRLAQQESNPTRKRELEQIAQNCTRVPEYGATTFWEACQTFWFIQSMLQIESSGHSISPGRFDQYMYPYLESDKSISREFAQELVDCCWIKLNDINKTRDEVSAQAFAGYAVFQNLCCGGQTEDGRDATNDLSYMCMEATAHVRLPQPSFSIRVWQGTPDEFLYRACELVRMGLGVPAMYNDEVIIPALQNRGISLRDARDYCIIGCVEPQAPHRTEGWHDAAFFNVAKVLEITLNNGRVGNKQLGPVTGELTQFTSMEDFYTAFQKQMAHFVHQLVEACNSVDIAHGERCPLPFLSALVDDCIGRGKSLQEGGAIYNFTGPQAFGVADTGDSVYAIQKQVFEDRKLSLSELKSALDANFGYPVGANPHTPAAKSSLNEQDIYDVVKRIIEQHGALDPAAIKNEVYRQLTSGSAAPVQSGTMSRHEEIRRILENTPCFGNDIDDVDLVARKCALIYCQEVEKYTNPRGGQFQAGIYPVSANVLFGKDVAALPDGRLAKEPLADGVSPRQGKDTLGPTAAANSVAKLDHFIASNGTLYNQKFLPSSLAGENGLRNFSGLIRHYFDKKGMHVQFNVIDRNTLIEAQKNPEQHQDLVVRVAGYSAQWVVLAKEVQDDIISRTEQQLS

SEQ ID NO: 121 - protein, 849 aa, Artificial Sequence
  n640757250_APECO1_2293/1-850
MLEKGFSNPTDRVVRLRNMILTAKPYVESERAVLATEAYKETEQLPAIMRRAKVVEKIFNQLPVTIRPDELIVGAVTINPRSTEICPEFSYDWVEKEFETMEHRIADPFVIPKKTAQELHEAFKYWPGKTTSALAASYMSEGTKESMASGVFTVGNYFFGGVGHVSVDYGKVLKIGFRGIINEVSRALESLDRTEPGYIKKEQFYNAVLISYNAAIRFAHRYAEEASRLAQQESNPTRKRELEQIAQNCTRVPEYGATTFWEACQTFWFIQSMLQIESSGHSISPGRFDQYMYPYLESDKSISREFAQELVDCCWIKLNDINKTRDEVSAAFAGYAVFQNLCCGGQTEDGRDATNDLSYMCMEATAHVRLPQPSFSIRVWQGTPDEFLYRACELVRMGLGVPAMYNDEVIIPALQNRGISLRDARDYCIIGCVEPQAPHRTEGWHDAAFFNVAKVLEITLNNGRVGNKQLGPVTGELTQFTSMEDFYTAFQKQMAHFVHQLVEACNSVDIAHGERCPLPFLSALVDDCIGRGKSLQEGGAIYNFTGPQAFGVADTGDSVYAIQKQVFEDRKLSLSELKSALDANFGYPVGANPHTPAAKSSLNEQDIYDVVKRIIEQHGALDPAAIKNEVYRQLTSGSAAPVQSGTMSRHEEIRRILENTPCFGNDIDDVDLVARKCALIYCQEVEKYTNPRGGQFQAGIYPVSANVLFGKDVAALPDGRLAKEPLADGVSPRQGKDTLGPTAAANSVAKLDHFIASNGTLYNQKFLPSSLAGENGLRNFSGLIRHYFDKKGMHVQFNVIDRNTLIEAQKNPEQHQDLVVRVAGYSAQWVVLAKEVQDDIISRTEQQLS

SEQ ID NO: 122 - protein, 832 aa, Artificial Sequence
  n638867180_Ecol1_01002098/1-832
MILTAKPYVESERAVLATEAYKETEQLPAIMRRAKVVEKIFNQLPVTIRPDELIVGAVTINPRSTEICPEFSYDWVEKEFETMEHRIADPFVIPKKTAQELHEAFKYWPGKTTSALAASYMSEGTKESMASGVFTVGNYFFGGVGHVSVDYGKVLKIGFRGIINEVSRALESLDRTEPGYIKKEQFYNAVLISYNAAIRFAHRYAEEASRLAQQESNPTRKRELEQIAQNCTRVPEYGATTFWEACQTFWFIQSMLQIESSGHSISPGRFDQYMYPYLESDKSISREFAQELVDCCWIKLNDINKTRDEVSAQAFAGYAVFQNLCCGGQTEDGRDATNDLSYMCMEATAHVRLPQPSFSIRVWQGTPDEFLYRACELVRMGLGVPAMYNDEVIIPALQNRGISLRDARDYCIIGCVEPQAPHRTEGWHDAAFFNVAKVLEITLNNGRVGNKQLGPVTGELTQFTSMEDFYTAFQKQMAHFVHQLVEACNSVDIAHGERCPLPFLSALVDDCIGRGKSLQEGGAIYNFTGPQAFGVADTGDSVYAIQKQVFEDRKLSLSELKSALDANFGYPVGANPHTPAAKSSLNEQDIYDVVKRIIEQHGALDPAAIKNEVYRQLTSGSAAPVQSGTMSRHEEIRRILENTPCFGNDIDDVDLVARKCALIYCQEVEKYTNPRGGQFQAGIYPVSANVLFGKDVAALPDGRLAKEPLADGVSPRQGKDTLGPTAAANSVAKLDHFIASNGTLYNQKFLPSSLAGENGLRNFSGLIRHYFDKKGMHVQFNVIDRNTLIEAQKNPEQHQDLVVRVAGYSAQWVVLAKEVQDDIISRTEQQLS

SEQ ID NO: 123 - protein, 847 aa, Artificial Sequence
  n637924274_RPC_1163/1-847
MIEKGFSKPTERVMRLKNVILNAKPFVESERAVLVTDAYKETEGLPAILRRAKAAEKIFNNLPVTIRADELIVGAITKRPRSTEICPEFSFDWVEKEFETMATRVADPFQIPKETAAELHEAFKYWPGKTTSDLASSYMSQEAKDCIAAGVFTVGNYFYGGVGHVCVDYGKVLKIGFRGIITEVVLAMEKLDRMDPGYIKKQQFYNAVIISYTAAINFAHRYAVKAEELAQTESNATRKAELLQIAKNCARVPEYGASNFYEACQSFWFLQALLQIESSGHSISPGRFDQYMYPFLAADKSISREFAQELIDCIWIKLNDVNKTRDGGSAQAFAGYAVFQNLCVGGQTEEGLDATNDVSFMCMEATAHVALPAPSFSIRVWQGTPDDFLYRACEVVRLGLGVPAMYNDEVIVPSLQNRGVSLRDARDYGIVGCVEPQAIHKTEGWHDAAFFNVAKVLEITLNNGRVGDKQVGPASGELLSFRCIDDVFAAFQKQIEYFVRYLVEADNCVDLAHGERCPLPFVSALVEDCIGRGKSLQEGGALYNFTGPQAFGVADTGDSVYAIQKNVFEDKKITLGELKAALDANFGRPVGESAHADAGTNYTEEQVFAAVKKVLNSSGSTDVSALKGKVYSALAGANGAKSGGASSSYDALHRLLEATPAFGNDIHEVDMVARRCAQIYCLEVEKYTNPRGGQFQAGIYPVSANVLFGKDVAALPDGRFAKAPLADGVSPRQGKDVNGPTAAANSVAKLDHFIASNGTLYNQKFLPAALAGDSGLQNFASLVRSYFDHKGMHVQFNVVDRQTLLDAQREPEKHNDLVVRVAGYSAQFVVLAKEVQDDIISRTEQTL

SEQ ID NO: 124 - protein, 846 aa, Artificial Sequence
  n637824991_Rru_A0903/1-846
MIEKGFSKPTDRVMRLKNEILNAKPYVESERAVLVTEAYKETEGLPAILRRAKAAEKIFNNLPVTIRNDELIVGAITKNPRSTEICPEFSYDWVEKEFDTMATRLADPFLIPKETAKELHDAFLYWPGKTTSDLASSYMSQEAKDCIASGVFTVGNYFYGGVGHVCVDYGKVLKIGFRGIITEVVQAMEKMDRMDPDYIKKQQFYNAVIIAYTAAINFAHRYAAKALELAQNEANPTRKAELLQIAQNCARVPENGATTFYEACQSFWFVQCLLQIESSGHSISPGRFDQYMYPFLCADKSIDKGFAQELVDCIWIKLNDVNKTRDEVSAQAFAGYAVFQNLCVGGQTEGGLDATNEISYMCMEATAHVRLPAPSFSIRVWQGTPDDFLHRACEVVRLGLGVPAMYNDEVIVPALQNRGVTLHDARNYGIVGCVEPQCIHKTEGWHDAAFFNVAKVLEITLNNGKAGGKQLGPVTGEFTSFRNMDDLYAAFQKQMAYFVHYLVEADNCVDLAHGERCPLPFVSALVDDCIGRGKSLQEGGAIYNFTGPQAFGVADTGDSVYAIQKNVFEDKKITLAEMKEALDANFGLPVGGSAPSAGGDFTEEQVFAAVRKVLSSNGSMDVSALKGEVYRTLSGQAAPAAGGSSTKYDAIRRLLDASPAFGNDIDDVDMVARECALIYCREVEKYTNPRGGQFQAGIYPVSANVLFGKDVAALPDGRLAKAPLADGVSPRPGQDVKGPTAAANSVAKLDHFIASNGTLYNQKFLPSALAGDAGLQNFASLVRSYFDHKGMHVQFNVIDRQTLLDAQLEPEKHNDLVVRVAGYSAQFVVLAKEVQDDIISRTEQTL

SEQ ID NO: 125 - protein, 844 aa, Artificial Sequence
  n640774280_Cbei_4061/1-844
MISKGFSKPTERVERLKRMIVDAIPYVESERAVLVTESYKETEGLSPILRRAKAVEKIFNNLPITIREDELVVGAITKNPRSTEICPEFSYDWVAKEFDTMGARVADPFQIPKETAAELSEAFKYWDGKTTSALADSYMSQEAKDCMANGVFTVGNYFYGGVGHICVDYGKILRKGFKGIIAEVIEAMSKMDKKDPDYIKKQQFYNAVVISYSAAINFAHRYAQKARDMAAAELNPTRKAELLQIAANCERVPENGATNFYEACQSFWFIQIMVQIESNGHSISPGRFDQYMYPYLKEDKNISKEFAQELVDCIWIKLNDINKTRDEISAQAFAGYAVFQNLCVGGQNEEGLDATNEISYMCMDATAHVKLPAPSFSIRVWQGTPDEFLLRACEVARLGLGVPAMYNDEVIIPALVNRGVTLRDARNYCIIGCVEPQCPNKTEGWHDAAFFNVAKVLEITLNNGKVGNKQLGPITGDITTFKSIDDFYAAFKKQMEYFVYYLVEADNCVDYAHAERAPLPFLSAMVDDCIGRGKSVQEGGAIYNFTGPQAFGIADTGDSVYAIQKHVFEDKTIEMDQLKAALDANFGHTGVNTVSTSNNNADVTEMQIYEAVKRILSNSGSIDISEIQSRISSEFTSPKTTVSGDFDNIRRLLESTPCFGNDIDEVDMVARKCAQIYCFEVEKYTNPRGGQFQAGVYPVSANVLFGKDVAALPDGRLAKTPLADGVSPRAGKDCAGPTAAANSVAKLDHFVASNGTLYNQKFLPSAVAGDTGLQNFASVIRSYFDHKGMHVQFNVIDKQLLLDAQKHPENYKDLVVRVAGYSAQFTVLAKEVQDDIINRTEHSL

SEQ ID NO: 126 - protein, 887 aa, Artificial Sequence
  n641407376_CLOBOL_07236/1-887
MRRRDALELMDGQIPETENYAIENKIKEDICMIAKGFTEPTERVKRLKRAIVDAIPYVESERAVLVTESYKETEGLSPIMRRAKAAEKIFNNLPITIHDDELVVGAITKNLRSTEICPEFSYDWVEKEFETMGTRVADPFQIPKDTAAELHEAFKYWEGKTTSALADSYMSQETKDCIANGVFTVGNYFYGGVGHVCVDYGKVLDIGFTGIIKQVIETMEKLDTSDPEYIKKKNFYEAIVITYTAAINFAHRYAAKAREMAASCPDPVRKAELLQIAANCDRVPERGATNFYEACQAFWFVQILLQIEANGHSISPGRFDQYMYPHLAADKNICPEFAQELVDCIWVKLNDVNKTRDEVSAQAFAGYAVFQNLIVGGQTEDGLDATNDVSYMCMEAVAHVALPAPSFSIRVHQNTPDEFLYRACEVTRLGLGVPAMYNDEVIIPALCNRGVSLADARSYCIIGCVEPQCPHKTEGWHDAAFFNIAKVLEITLNNGKVGDKQLGPQTGDMTSFTSIEDIFAAYKKQMEYFVYHLAEADNCVDFAHAERAPLPFLSALVDDCIGRGKSVQEGGAIYNFTGPQAFGVADSGDSLCAIKKHVFESKEVTMAQLKEAMANNFGYACNASAPAATADECTDEARIYEAVKRILSNNGSINLADLQAQLAGPAQACRWPSPAEPAKTEPACVNPDYAHIKRLMENTPWFGNDIDEVDMIARRCGQIYSYEVEKYTNPRGGQFQAGCYPVSANVLFGKDVSALPDGRLAKTPLADGVSPRQGKDTNGPTAAAMSVAKLDHANYSNGTLYNQKFLPDALAGDEGLKRFASVVRAYFDHKGMHVQFNVIDRATLLAAQEHPEDYKDLVVRVAGYSAQFTVLAKEVQDDIISRTEQTF

SEQ ID NO: 127 - protein, 850 aa, Artificial Sequence
  n639733029_NT01CX_0498/1-850
MNDILARNYSSIPKERINILIEDLYSVTPEIEADRAVLITESFKETESMPMVIRRAKALEKILSEMPIVIRDSELIVGNLTKKPRAAQIFPEFSNKWLLDEFDRLANRKGDVFLISEDTKDKLREVFKYWDGKTTNEFATEIMFDETKEAMDEGVFTVGNYYFNGVGHICVDYAKVLSKGFNGIIQEVQEERKKADKGDPNYIKKDQFLTSVEITCKAAVKFAKRFGEEAKTLASRTMDSKRREELLQIAHNCEWVPANPARNFYEALQAFWFVQAIIQIESNGHSISPMRFDQYMYPYFKNDIESGRIDMSRAQELLDCLWVKFNDVNKVRDEGSTKAFGGYPMFQNLIVGGQTIYGEDATNELSFMCLEATAHTKLPQPSISIRGWNKTPDELLFKAAEVSRLGLGMPAYYNDEVIIPSLLNRGLSMEDARDYGIIGCVEPQKGGKTEGWHDAAFFNMAKVLEITMNNGMSNGKQLGPKTGDVTLFNSFEEFMNAYREQMKYFVKLLANADNCVDVAHGMRAPLPFLSSMVYDCIGKGKSLQEGGAHYNFTGPQGVGVANTADSLEVIKKLVFEERLVSMGDLKEALDTNFGECNSSNSLNLNSINNINPENLNRETIMAVIEKLLFKESNISVNNLNSNINLGNYQGKESLRQMLINRAPKYGNDIDEVDNLAREAALIYCKEVEKYTNPRNGKFQPGLYPVSANVPMGAQTGATPDGRKAGEPLADGVSPVSGRDQNGPTAAVNSVAKLDHAIASNGTLFNQKFHPSALQGEAGLRNLSALVRTFFENKGMHVQFNVVSREMLLDAQKNPEKYKSLVVRVAGYSAHFTSLDKSIQDDIIKRTEHQL

SEQ ID NO: 128 - protein, 847 aa, Artificial Sequence
  n639814465_Sputw3181_0427/1-847
MSQLSQAFGEPTDRIRALREQILDTTPCIETDRARLITESYKETESLPMIIRRAKALEKILAELPVTIRAGELIVGSLTVTPHSTQIYPEYSNRWLQDEFDRLNLRKGDRFTITDEAKQQLDSVFGYWEGKTTNELATSYMLPETLDCMAENVFTVGNYYFNGVGHIAVDYARVLARGYKGIIQDVVAAMASADKKDPAFLKKESFYKAVIISCNAAINFAHRYAVKARTLAEQASPVRKKELLKIAEICDKVPENGASNFYEACQSFWFAHAIIQLESNGHSISPARFDQYMYPYLEKDSSLSEEQAQELLDCLWLKFNDVNKVRDEGSTKGFGGYPMFQNLIVGGQTSGGQDATNRLSFMAMTATAHVRLHEPSLSVRVWSKSPDDLLLKACEVSRLGMGIPAYYNDEVVIPALINRGLTLEDAREYGIIGCVEPQRPGKTEGWHDAAFYNMSKVLEITLNNGRCGDKQLGPKTGELDSFQSIEDIIEAYRKQNEYFVYHLAMADNSVDLAHMERAPLPFLSCMVDDCISRGKSVQEGGAHYNFTGPQGVGVANVGDSLMAIKRLVFEEGQLSLGHLKEALDANFGVSGGIEKPDTIATESTPKQDATYELVLEAVKKVLGESGALALTSLNSNPPEPVKGANAGLTAVRQLLINGAPKFGNDIDEVDMLARTGAEIYCREVEKYTNPRGGLFQAGLYPVSANVALGESVGATPDGRLAGQPLPDGVSPSRGMDTKGPTAAANSVAKLDHFLASNGTLFNQKFHPAALKGDEGLYHLAALLRGYFDQKGMHVQFNVIDRNTLLAAQKEPEKYRDLVVRVAGYSAQFVSLDKSVQDDIILRTEHVF

SEQ ID NO: 129 - protein, 847 aa, Artificial Sequence; identical to SEQ ID NO: 128
  n640497328_Sputcn32_0208/1-847

SEQ ID NO: 130 - protein, 330 aa, Artificial Sequence
  n2503621166_Isop_1633/1-330
MSDYPAVNEWKTRQFMCEVGRRIYAKGFAAANDGNISFRLSEDRVLCSPTRVSKGFMKPDDLCIVDLDGVQISGKRKRSSEILLHLTIMKTRPDVRAVVHCHPPHATAFAVAHEPIPKCTMPEFEVFLGEVAISPYETPGGQSFADTVIPYVKDTDTILLANHGTVTCGTDLEDAYFKTEIIDAYCRILILARQLGRVQYYPDEKAAELIRLKPNLGIRDVRLELGLENCDLCGNSLFREGYSDFKPEPYAFRHPRLGGDATGIGPVAGPHSTNANANVNANASPPIQVQPGSPEFEQMVQMITDEIMGHLAGRSTSVSASAAASNPGGC

SEQ ID NO: 131 - protein, 303 aa, Artificial Sequence
  n646787467_Plim_1747/1-303
MTTANKWNSGINDRKLKELICEIGRRVYNKGFAAANDGNISIRVGENEVLCSPTMICKGFMTPDDICAVDLEGGQIAGKRKRTSEILLHLAIMKHRPDVKAVVHCHPPHATAFAVAREPIPQCILPEIEVFMGEVPIAPYETPGGHAFANTVVPFLKGTNTIILTNHGTVSFGANLEEAYWKTEILDAYCRILLLSKQLGRVEYLNERESVELLDLKKKLGFDDPRFHVENCDLCGNSAFREGYKDAQPQPAAFEPAPYYPGYLERQKSTPAPAAAPSAAAAPVDTEMLVKMITEQVMAALKK

SEQ ID NO: 132 - protein, 322 aa, Artificial Sequence
  n641110466_PM8797T_14741/1-322
MKFTETSLKLNPLTEITFFLTFGAKTMSNQWNSGIHDRKLKEEICEIGRRVYNKGFAAANDGNISIRVGENEVLCSPTMICKGFMKPDDICAVDLDGNQIAGTRKRTSEILLHLAIMKERPDVKAVVHCHPPHATAFAVAREPIPQCVLPEVEVFMGEVPMAPYETPGGQKFADTVVPFLKGGTNTIILTGHGTVTFGKSLEDAYWKTEILDAYCNILLLSKQLGRVTYFTENETRELLDLKKKLGFDDPRFHVEDCDLCGNSAFRDGYKEGIPQQKSFEPAPSYPGYLSKPSTQATPATNNGDSDQLIKAITDQVMSALGK

SEQ ID NO: 133 - protein, 287 aa, Artificial Sequence
  n637434385_RB2568/1-287
MQNIHKIKQDMCDIGRRIYNRQFAAANDGNITVRVSENEVLCTPTMHCKGYLTPDDISMIDMTGKQIAGRKKRSSEALLHLEIYKQRADIKSVVHCHPPHATAFAIAREPIPQCILPEVEVFLGDVPITKYETPGGQAFADTIIPFVEKTNVMILANHGTVSYGESVERAYWWTEILDSYCRMLLLAKQLGNVSYLDETKSRELLELKDKWGFKDPRNTSEYEDCDICANDIFRDSWKDSGVERRAFAPPPPIKTSGSASSAPAGVDEEQLVKLITNEVMRQMKASS

SEQ ID NO: 134 - protein, 287 aa, Artificial Sequence
  n638981608_DSM3645_04920/1-287
MMNVHRIKQDMCEIGRRIYNKGFAAANDGNITVRVSENEVLCTPTMQSKGFLKPEDIATIDMTGKQIAGSKPRSSEALLHLEIYQRRADIKSVVHCHPPHATAFAIAREPIPQCVLPEVEVFLGDVPITKYETPGGKAFAETILPFVDKTNIILLANHGTVSYGETVERAYWWTEILDAYCRMLILAKQLGRVEFFSEEKERELLDLKQRWGWSDPRNTEEYKDCDICANDIFRDSWKDSLIERKAFPAPPAMGPNANKAAAPVTGDQEALIQAITSRVMAELSKRS

SEQ ID NO: 135 - protein, 287 aa, Artificial Sequence
  n646480649_Psta_3288/1-287
MANIHKLKQDICEIGRRLYNKGFAAANDGNITIRVSENEVLVTPTMHSKGFLKPEDICMMDMSGKQIGGTKKRSSEALLHLEIFRERPEVKSVVHCHPPHATAFAIAREPIPQCVLPEVEVFLGDVPITMYETPGGKEFAETVLPFVKKTNVIILANHGTVSYGDNVEQAYWWTEILDAYCRMLMLAKDLGRVNYFSEKKERELLELKDKWGWKDPRNTPEYKDCDICANDIFRDSWKQSGVERKAFEAPPPMAPSAKKEAAPAAAGDQEALVRLITERVLAELSKK

SEQ ID NO: 136 - protein, 332 aa, Artificial Sequence
  CLOSTASPAR_02209/1-332
MMTIQGMKYKSDFEAKKAILDIGRRMYAKGFVASNDGNISCRVGPNTIWTTPTGVSKGFMTQDMLVKMDLDGKVLMGRLKPSSEIKMHLRVYQENPRLQAVTHAHPPMATCFAIAGQPLDAAILTEAILSLGTIPVARYATPGTQEVPDSIAPFVNHYNGVLLANHGALTWGDDIYQAFYRLESVEYYATILMYTGNIIGQQNHLSCEQVDRLLEIRKNMGITGGGVPPCMNGGQLTKVCESCAAAGEKTAAAGTELAGGSCGGCAAAGGTQTGPQAPLKGVTPLVRPGDAGKMPGGGLGAGSGSPSTGSGPADKDALIAEIVRRVVVQLKA

SEQ ID NO: 137 - protein, 291 aa, Artificial Sequence
  n642204486_ANACOL_01089/1-291
MKNMGGSIKMRNMGEYMGDYEAKQLILEVGRRMYNKNFVAANDGNISCKVGDNELWTTPTGVSKGYMTEDILVKVDLDGNILRGSTKPSSELKMHLRVYRENPQVKSVVHAHPPVATSFAIAGIPLSRAILPEAVVQLGEVPVAPYAAPGTQEVPDSIAPFCKTHNGVLLANHGALTWGKDPMQAYFRMESLEYYALVTMYTGSIIGQANELSCEQIDQLVDTRTRLGISTGGRPVCQNVGKDGVPACMEQKKCGGQCTHGGQPPAGADAGTVAMEDIVDIVRQVMARTKR

SEQ ID NO: 138 - protein, 260 aa, Artificial Sequence
  Bacillus_selenitireducens_646852828/1-260
MATAKYLSDFEAKKMICEIGDRIYKKNFVAANDGNISVKVGDNTIWTTPTGVSKGFMRPDMMVKMNLDGKILQGKMKPSSEVKMHLRAYKENTEIRSVVHAHPPVATSFAIAGVELNRPISPEAVVLLGTVPIAEYATPGTEEVPESIAPYCNTHNAVLLANHGALTWGKDIIEAYYRMESLEHYALMTMYSTNIIQKTNELNCDQISDLMGIRSKLGIHSGGTPSCQPERQETKKDVDIEAIVAAVTQEVIGKLQERRN

SEQ ID NO: 139 - protein, 284 aa, Artificial Sequence
  n644206069_CLOSTMETH_00022/1-284
MVSAYEIKKEICEIGRRIYMNGFVAANDGNISVKINDNEFYCTPTGVSKGFMTPDMIIKVDGQGNKIEGKLNPSSEFKMHLKVFQERPDVNAVVHAHPPIATAHAVCNIPLDTYIMPEAVIFLGTVPICEYGTPSTMEIPDSLAPYIQSHDAFLLKNHGALTVGNTLMKAYFNMESTEYFAKVSMYCRQLGGAQQLDCSQINRLLELREEFKAPGKHPGCPQCQVLPAEAVPVNTANPDGTQRRQPAAVIPGEIPAGVAPAAAAPSDNDLIAEITRKVLAQLGK

SEQ ID NO: 140 - protein, 279 aa, Artificial Sequence
  n644367789_GCWU000342_00652/1-279
MVNEYELKKQICDIGKRIYNRNMVAANDGNISVKLNDHEFLCTPTGVSKGFMTPDYICRVNEKGEVIQANPGFKPSSEIKMHMRVYAKRPDVGSVVHAHPVYATSFAIAGIPLTQPIMPEAVIALGCVPIAEYGTPSTMEIPDNVEKYLPYYDAVLLESHGALTWSTDLLSAYLKMESVEFYAQLLYQSKMLGGPKEFDQKTVERLYEIRRQMGLPGKHPANLCQNKDGHNCHNCGLHQEIPGMPASGATTGSITSTPKEPAPEVIAEITKRVLEQLGK

SEQ ID NO: 141 - protein, 260 aa, Artificial Sequence
  n644270208_ROSEINA2194_01705/1-260
MALNEYEIKKMMCDVGKRIYDRNMVAANDGNISVKLNDNEFLCTPTGVSKGFMTPEYICKVDAQGNVIQANKGFKPSSEIKMHMRVYAKRPDVGAVVHAHPTFATSFAIAGIPLTQPIMPEAVIALGCVPIAPYGTPSTMEIPDAVEPYLEHFDAVLLESHGALTWSTDLMAAYMKMESVEFYAELLYKAKQLGGPKEFDKEQIAKLYEIRRKMGLPGRHPANLCQNKGKENCHNCGGGCSSSAQVDDNKELVAAITKKY

SEQ ID NO: 142 - protein, 289 aa, Artificial Sequence
  n641004274_RUMOBE_00095/1-289
MVNEFEIKKQICDIGRRIYNRNMVAANDGNISVKLNDNEFLCTPTGVSKGFMTPEFICKVDAQGNVIQANPGFKPSSEIKMHMRVYQKRPDVGSVVHAHPIYATSFAIAGIPLTQPIMPEAVIALGCVPIAEYGTPSTMEIPDNLEKYLPYFDAVLLENHGALTWSTDLNAAYMKMESVEFYAQLLYQSKLLGGPKEFDKENIKKLYEIRRKFGMPGKHPANLCQNKDGVNCHNCGGACHSQDYKQFPGYQYDFVGSETKAEAPAATGAADAELVANITKQVMAQLGMK

SEQ ID NO: 143 - protein, 269 aa, Artificial Sequence
  n641052370_RUMGNA_01020/1-269
MQNEYEIKKEMCEIGKRVYNRGMVAANDGNFSVRISENEVLCTPTGVSKGFMTPDYICKVDLDGNVLQANKGFRPSSEIKMHLRVYKERPDVKSVVHAHPLYATTFAIAGIPLTQPIMPEAVIALGCVPIAKYGTPSTVEIPDAVSEHLQYFDAVLLENHGALTYSDSLLNAYHKMESVEFYARLLWQTMQIGGPQELNKEQVEKLYEIRRQMGLSGKHPANLCPNAKAGKPSCHSCGGGCGAAKTEETPDADLVASITKKVMDQLGLN

SEQ ID NO: 144 - protein, 264 aa, Artificial Sequence
  n641292282_Cphy_1177/1-264
MNEYEVKKEICEIGRRIYNKGMVAANDGNISVKLNENEFLCTPTGVSKGFMTPDYICKVDKDGKVLQANGIYKPSSEIKMHMRVYQERPDVNAVVHAHPMYATSFAIAGIPLTQPIMPEAVISLGCVPIAEYGTPSTDEIPDAISKYIQHFDSVLLANHGALSFSDSLLNAYFKMESTEFYAQLLYQAKVLGGPKELSNSQVQRLYELRREFGLKGKHPANLCSNTKEGKASCHCCGEECKSGGVDNADLVASITRKVMEQLGL

SEQ ID NO: 145 - protein, 12 aa, Artificial Sequence
  C-terminal region of CcmN  of Synechococcus elongatus PCC7942
GKEQFLRMRQSM

SEQ ID NO: 146 - protein, 11 aa, Artificial Sequence
  C-terminal region of CcmN  of Synechococcus elongatus PCC7942
KEQFLRMRQSM

SEQ ID NO: 147 - protein, 12 aa, Artificial Sequence
  C-terminal region of CcmN  of Synechocystis PCC 6803
GQVYINQLLMTL

SEQ ID NO: 148 - protein, 11 aa, Artificial Sequence
  C-terminal region of CcmN  of Synechocystis PCC 6803
QVYINQLLMTL

SEQ ID NO: 149 - protein, 16 aa, Artificial Sequence
  S. typhimurium
EKLLRQIIEDVLRDMK

SEQ ID NO: 150 - protein, 11 aa, Artificial Sequence
  S. typhimurium
EKLLRQIIEDV

SEQ ID NO: 151 - protein, 16 aa, Artificial Sequence
  S. termitidis
EKQLKDIIAGVIKEIQ

SEQ ID NO: 152 - protein, 11 aa, Artificial Sequence
  S. termitidis
EKQLKDIIAGV

SEQ ID NO: 153 - protein, 16 aa, Artificial Sequence
  L. brevis
ENLLRNIIRDVIAETQ

SEQ ID NO: 154 - protein, 11 aa, Artificial Sequence
  L. brevis
ENLLRNIIRDV

SEQ ID NO: 155 - protein, 14 aa, Artificial Sequence
  S. typhimurium
DAIESMVRDVLSRM

SEQ ID NO: 156 - protein, 11 aa, Artificial Sequence
  S. typhimurium
TDAIESMVRDV

SEQ ID NO: 157 - protein, 14 aa, Artificial Sequence
  S. termitidis
VMIKNMVKEILNNI

SEQ ID NO: 158 - protein, 11 aa, Artificial Sequence
  S. termitidis
EVMIKNMVKEI

SEQ ID NO: 159 - protein, 14 aa, Artificial Sequence
  L. brevis
SEIDDLVAKIVQQI

SEQ ID NO: 160 - protein, 11 aa, Artificial Sequence
  L. brevis
MSEIDDLVAKI

SEQ ID NO: 161 - protein, 16 aa, Artificial Sequence
  S. typhimurium
QKQIEEIVRSVMASMG

SEQ ID NO: 162 - protein, 11 aa, Artificial Sequence
  S. typhimurium
QKQIEEIVRSV

SEQ ID NO: 163 - protein, 16 aa, Artificial Sequence
  Sebaldella termitidis
ERELREIIGKVIDEMG

SEQ ID NO: 164 - protein, 11 aa, Artificial Sequence
  Sebaldella termitidis
ERELREIIGKV

SEQ ID NO: 165 - protein, 15 aa, Artificial Sequence
  EutE C-terminal peptide (Cphy_2642), Left sequence
NTELVEEIVKRIMKQ

SEQ ID NO: 166 - protein, 11 aa, Artificial Sequence
  EutE C-terminal peptide (Cphy_2642), Right sequence
TELVEEIVKRI

SEQ ID NO: 167 - protein, 15 aa, Artificial Sequence
  Inter-domain peptide R. palustris BisB18 (RPC_1163), Left sequence
AGTNYTEEQVFAAVK

SEQ ID NO: 168 - protein, 11 aa, Artificial Sequence
  Inter-domain peptide R. palustris BisB18 (RPC_1163), Right sequence
EEQVFAAVKKV

SEQ ID NO: 169 - protein, 18 aa, Artificial Sequence
  Inter-domain peptide C. phytofermentans (Cphy_1174), Left sequence
ILAQQITVQIVKELKERG

SEQ ID NO: 170 - protein, 11 aa, Artificial Sequence
  Inter-domain peptide C. phytofermentans (Cphy_1174), Right sequence
IILAQQITVQI

SEQ ID NO: 171 - protein, 16 aa, Artificial Sequence
  Fuculose phosphate aldolase from C. phytofermentans, Left sequence
NADLVASITRKVMEQL

SEQ ID NO: 172 - protein, 11 aa, Artificial Sequence
  Fuculose phosphate aldolase from C. phytofermentans, Right sequence
ADLVASITRKV

SEQ ID NO: 173 - protein, 16 aa, Artificial Sequence
  Aldehyde dehydrogenase from C. kluyveri, Left sequence
DNEDVQAIVKAIMAKL

SEQ ID NO: 174 - protein, 11 aa, Artificial Sequence
  Aldehyde dehydrogenase from C. kluyveri, Right sequence
NEDVQAIVKAI

SEQ ID NO: 175 - protein, 16 aa, Artificial Sequence
  Fuculose phosphate aldolase from P. limnophilus, Left sequence
TEMLVKMITEQVMAAL

SEQ ID NO: 176 - protein, 11 aa, Artificial Sequence
  Fuculose phosphate aldolase from P. limnophilus, Right sequence
EMLVKMITEQV

SEQ ID NO: 177 - protein, 17 aa, Artificial Sequence
  Fuculose/rhamnose phosphate aldolase from O. terrae PB90-1, Left sequence
VEALVQRLTEEILRQLQ

SEQ ID NO: 178 - protein, 11 aa, Artificial Sequence
  Fuculose/rhamnose phosphate aldolase from O. terrae PB90-1, Right sequence
EALVQRLTEEI

SEQ ID NO: 179 - protein, 15 aa, Artificial Sequence
  Aldehyde dehydrogenase from O. terrae PB90-1, Left sequence
ETLVRSVVEEVVRAF

SEQ ID NO: 180 - protein, 11 aa, Artificial Sequence
  Aldehyde dehydrogenase from O. terrae PB90-1, Right sequence
ETLVRSVVEEV

SEQ ID NO: 181 - protein, 17 aa, Artificial Sequence
  Aldehyde dehydrogenase (Cphy_1416) from C. phytofermentans, Left sequence
MEDARDLLKQILQALSK

SEQ ID NO: 182 - protein, 11 aa, Artificial Sequence
  Aldehyde dehydrogenase (Cphy_1416) from C. phytofermentans, Right sequence
MEDARDLLKQI

SEQ ID NO: 183 - protein, 15 aa, Artificial Sequence
  Aldehyde dehydrogenase (Cphy_1428) from C. phytofermentans, Left sequence
NEKLAALVEKEIVLA

SEQ ID NO: 184 - protein, 12 aa, Artificial Sequence
  Aldehyde dehydrogenase (Cphy_1428) from C. phytofermentans, Right sequence
NEKLAALVEKEI

SEQ ID NO: 185 - protein, 14 aa, Unknown
  Unknown glycyl radical enyzme (Cphy_1417) from C. phytofermentans, Left sequence
IREFSNKFVEATKN

SEQ ID NO: 186 - protein, 11 aa, Unknown
  Unknown glycyl radical enyzme (Cphy_1417) from C. phytofermentans, Right sequence
IREFSNKFVEA

SEQ ID NO: 187 - protein, 20 aa, Artificial Sequence
  Aldehyde dehydrogenase from M. smegmatis, Left sequence
LDALRAELRALVVEELAQLI

SEQ ID NO: 188 - protein, 11 aa, Artificial Sequence
  Aldehyde dehydrogenase from M. smegmatis, Right sequence
LDALRAELRAL

SEQ ID NO: 189 - protein, 15 aa, Artificial Sequence
  Aldehyde dehydrogenase from H. ochraceum, Left sequence
EDRIAEIVERVLARL

SEQ ID NO: 190 - protein, 11 aa, Artificial Sequence
  Aldehyde dehydrogenase from H. ochraceum, Right sequence
EDRIAEIVERV

SEQ ID NO: 191 - protein, 18 aa, Artificial Sequence
  CcmN of S. elongatus PCC7942
VYGKEQFLRMRQSMFPDR

SEQ ID NO: 192 - protein, 39 aa, Artificial Sequence
  C-terminal residues from CcmN Syenchococcus elongatus PCC7942
VSSSEPAGRSPQSSAIAHPTKVYGKEQFLRMRQSMFPDR

SEQ ID NO: 193 - protein, 8 aa, Artificial Sequence
  CcmN of S. elongatus PCC7942 linker
GSGSGSGS

SEQ ID NO: 194 - protein, 41 aa, Propionibacterium acnes J139
MTTAVPPTSTQLCGVFADIDAAVAAAHKAFLAFSDCSLAQR

SEQ ID NO: 195 - protein, 65 aa, Fusobacterium ulcerans ATCC 49185
MNLEANNMDEIVALIMKELKKTDIKAGCQSCESPKNGVFSSMDEAIAAAKKAQEILFSSRLEMRE

SEQ ID NO: 196 - protein, 97 aa, Escherichia coli CFT073
MNDIEIAQAVSTILSKFTKATPDEAPATSEAARVDGLDEIVAKALAQHSSVRDASAISQVAKWAMASTGAFDTMDEAISAAVLAQVQYRHCSMQDRA

SEQ ID NO: 197 - protein, 97 aa, Pectobacterium wasabiae WPP163
MNDLEITQAVSRALSKYTKTTPEAQEHSGPSATPAPDRDNIEAIVASALARRAGAEPAADQTSGNGAFATMDEAIAAAQHAQHAQIQYRHCSMQDRT

SEQ ID NO: 198 - protein, 64 aa, Listeria monocytogenes 10403S
MESLELEKLVKKVLLEKLAEQKDAPVKTMTKGAKSGVFDTVDEAVQAAVIAQNSYKEKSLEERR

SEQ ID NO: 199 - protein, 59 aa, Shewanella sp W3-18-1
MNTTELENMIRNILADNLKGTATAPGNIQHTIFARVEDAITASYDAYKKYMAEPLALRT

SEQ ID NO: 200 - protein, 62 aa, Tolumonas auensis DSM 9187
MNNTELESLIRTILTEQLTPSATDTPACTASSVALFDDVDSAICAAHAAFLRYQEAPLKTRS

SEQ ID NO: 201 - protein, 55 aa, Yersinia frederiksenii ATCC 33641
MNINNLESLIRTILTEQLTPATTSASSAIFASVDEAVNAAHSAFLRYQQPMKTRS

SEQ ID NO: 202 - protein, 57 aa, Klebsiella pneumoniae 342
MNTAELETLIRTILSEKLAPAPVSQEQQGIYRDVGSAIDAAHQAFLRYQQCPLKTRS

SEQ ID NO: 203 - protein, 60 aa, Salmonella typhimurium LT2
MNTSELETLIRTILSEQLTTPAQTPVQPQGKGIFQSVSEAIDAAHQAFLRYQQCPLKTRS

SEQ ID NO: 204 - protein, 60 aa, Salmonella enterica Paratyphi B str. Sp87; identical to SEQ ID NO: 203

SEQ ID NO: 205 - protein, 57 aa, Citrobacter koseri ATCC BAA 895
MNTSELETLIRNILSEQLAPAQAETQGHGIFQSVGEAIDAAHQAFLRYQQCPLKTRS

SEQ ID NO: 206 - protein, 229 aa, Synechococcus sp. JA-3-3Ab; identical to SEQ ID NO: 66

SEQ ID NO: 207 - protein, 219 aa, Synechococcus sp. JA-2-3B′a(2-13); identical to SEQ ID NO: 67

SEQ ID NO: 208 - protein, 304 aa, Trichodesmium erythraeum; identical to SEQ ID NO: 51

SEQ ID NO: 209 - protein, 235 aa, Synechococcus sp PCC7002; identical to SEQ ID NO: 64

SEQ ID NO: 210 - protein, 244 aa, Cyanothece sp PCC8801; identical to SEQ ID NO: 69

SEQ ID NO: 211 - protein, 244 aa, Cyanothece sp PCC8802; identical to SEQ ID NO: 55

SEQ ID NO: 212 - protein, 240 aa, Crocosphaera watsonii
MPLPLIQPPSRSEVSVIGEVIIHQGAVVAPGTILQAAPNCRIVIHSGACIGMGTLINAYQGDIEIESGAMLGAGVLIVGQSKISQNVCLGSCTTVINSSIESGTTIEAGTLIGDTSRQFSEEETKAPKQIKAENNGSSENGHLIADNNQKDNLPQQSEEKKPEFVEEIEDLWADTPPKVEEVTEIPEIPTKPDTPTETKNAPVVGQVYINQLLCTLFPDRQAFNQSQNNSASKDPPGKNK

SEQ ID NO: 213 - protein, 241 aa, Cyanothece sp CCY0110; identical to SEQ ID NO: 57

SEQ ID NO: 214 - protein, 240 aa, Cyanothece sp ATCC51142; identical to SEQ ID NO: 58

SEQ ID NO: 215 - protein, 186 aa, Acaryochloris marina MBIC11017; identical to SEQ ID NO: 68

SEQ ID NO: 216 - protein, 262 aa, Cyanothece sp PCC 7822; identical to SEQ ID NO: 47

SEQ ID NO: 217 - protein, 220 aa, Microcystis aeruginosa; identical to SEQ ID NO: 59

SEQ ID NO: 218 - protein, 241 aa, Synechocystis sp PCC 6803; identical to SEQ ID NO: 65

SEQ ID NO: 219 - protein, 201 aa, Gloeobacter violaceus; identical to SEQ ID NO: 48

SEQ ID NO: 220 - protein, 224 aa, Lyngbya sp PCC 8106; identical to SEQ ID NO: 54

SEQ ID NO: 221 - protein, 248 aa, Nostoc sp PCC 7120; identical to SEQ ID NO: 61

SEQ ID NO: 222 - protein, 248 aa, Anabaena variabilis; identical to SEQ ID NO: 62

SEQ ID NO: 223 - protein, 265 aa, Nodularia spumigena; identical to SEQ ID NO: 60

SEQ ID NO: 224 - protein, 257 aa, Nostoc punctiforme; identical to SEQ ID NO: 63

SEQ ID NO: 225 - protein, 231 aa, Cyanothece sp PCC 7425; identical to SEQ ID NO: 53

SEQ ID NO: 226 - protein, 220 aa, Thermosynechococcus elongatus; identical to SEQ ID NO: 52

SEQ ID NO: 227 - protein, 161 aa, Synechococcus elongatus PCC 6301; identical to SEQ ID NO: 49

SEQ ID NO: 228 - protein, 161 aa, Synechococcus elongatus PCC 7942; identical to SEQ ID NO: 49

SEQ ID NO: 229 - protein, 117 aa, Synechococcus sp. JA-3-3Ab
IPPRSLVGDPTYPSRQEAEVGMASEAEPVSAAAPQEPIEPPEETLPEPTPPSPPDSPLAQVEKQTRRWQEAAEQTQENSRSPKTRKLNGIPGYSELDRLLGKIYPYRQILSSGGGQS

SEQ ID NO: 230 - protein, 113 aa, Synechococcus sp. JA-2-3B′a(2-13)
IPPGSLIGDPTYGFDLQEAGGSKPIPAEPSPAAVEMAPEMSPEPSPPPSSPVANVEKQTRRWQEAAEQTQEKSGSPRTKTRNLNGIPGHWELDRLLSKIYPHRQVLSSGDSRL

SEQ ID NO: 231 - protein, 199 aa, Trichodesmium erythraeum
VLPASIIGNSGRQFSDNSTISLPDQDSNQSYLFSNETQESSYSLNLANTASSTEETSTETEKANTQLPLANTSLPAEETPTETEKANTQLPLANTSLPAEETPTETEKANTQLPLANTSLPVEETPTETEKANTQLPLANTSLPVEETPTETEKANTQLQEESPPNIDAQIYGKEYVNKIMQTLFPYKNSLSSHPDDED

SEQ ID NO: 232 - protein, 133 aa, Synechococcus sp PCC7002
LPPQSLIGDPSRQETTASYQTQPPKPANQSTTQPLDPWQAEDTTNQTATTFSPPGRSPTSSSNRPNVQPPPEAGSPPTETPNTEVMPTVPESKESLESGEKTPVVGQVYINQLLMTLFPHQNSLNTPNQPDEP

SEQ ID NO: 233 - protein, 139 aa, Cyanothece sp PCC8801
IAPGSLLGDQSRQVTAETSEPTKELKSENNGSVTNNNSSISNKNNIFSKVQPTEDKKPNFVEEMQDLWAEPEPEVEPIAEVSPPPKPSVDPIPEVVAEPKPSPEPQNAPVVGQIYINQLLYTLFPERQAFNRSQNGSSS

SEQ ID NO: 234 - protein, 139 aa, Cyanothece sp PCC8802
IAPGSLVGDQSRQVVSETSPSTKEIKSENNGSVANNNGSTFNNDHIASKVASTEDKKPTFVQEMEDLWAEPEPEVEPVAEVSPPPKPSVEPIPEVLTQPKPSPDPQNAPVVGQIYINQLLYTLFPERQAFNRSQNGSSS

SEQ ID NO: 235 - protein, 135 aa, Crocosphaera watsonii
IEAGTLIGDTSRQFSEEETKAPKQIKAENNGSSENGHLIADNNQKDNLPQQSEEKKPEFVEEIEDLWADTPPKVEEVTEIPEIPTKPDTPTETKNAPVVGQVYINQLLCTLFPDRQAFNQSQNNSASKDPPGKNK

SEQ ID NO: 236 - protein, 136 aa, Cyanothece sp CCY0110
IEAGSLMGDTSRQFQEKESQSPPAIKADDNGFGDNGHLTANDQKKASQTDTTNHNKPGFVEEMEDLWADSEPEIEEVTKIPEIPEIPTKSNSPADKNNAPVVGQVYINQLLCTLFPDRQAFNQAQNNPPSQDENNE

SEQ ID NO: 237 - protein, 135 aa, Cyanothece sp ATCC51142
IEAGSLVGDTSRQFPEKESASSQGIKEDNNGFSDDRHLTANTQNKESQTNKNSSNKPEFVQEMEDLWADPEPEIEEVTEIPEIPTKPNAPADNNNAPVVGQVYINQLLCTLFPDRQAFNQSQNHSASDNSANNNK

SEQ ID NO: 238 - protein, 81 aa, Acaryochloris marina MBIC11017
IPEGSLMGDASRSTIDESPNRSPFDDSLPSTPVNTAWPSSPPPIPNPTPASPPQRQSHVIGRAYVTQMLQVLFARNSSPYP

SEQ ID NO: 239 - protein, 157 aa, Cyanothece sp PCC 7822
ITAGSLIGDTTRSFTPEPETTNGNGAKQPDFSKLNRPEKIQEELPPIVASPPKEHPSVVELESDPWTIDPIDDDQSSSKSDSVLSNTQVHEPEPATETRVEVTPQPPDLEPTEQSKQAPVVGQIYINQLLLTLFPERRFFQNLDQKNQSLHSEENSQ

SEQ ID NO: 240 - protein, 115 aa, Microcystis aeruginosa
VPSGSLIGDTSRQITIEVSATRSEPERPPLPEPEPVVSQVSPVPSVEEVVAETVASPWDSEEMVAEASPAETREQASTTNRPNQASVVGKVYINQLLVTLFPERHRFNGNNNHNS

SEQ ID NO: 241 - protein, 136 aa, Synechocystis sp PCC 6803
IPPGSLLMNRVADVQTVGASSPTTDSVTEKKSPSTANPIAPIPSPWDNEPPAKGTDSPSDQAKESIARQSRPSTAEAAEQISSNRSPGESTPTAPTVVTTAPLVSEEVQEKPPVVGQVYINQLLLTLFPERRYFSS

SEQ ID NO: 242 - protein, 98 aa, Gloeobacter violaceus
VPPNSLVGQAGRSAEAFPTAAAQPYVVPAAPAPRDPNQALAAGFDPPVQAALPEPQGGIVQNGQPPVAGKAYLERLRLSLFPHNAPLQNPDSATGGGA

SEQ ID NO: 243 - protein, 119 aa, Lyngbya sp PCC 8106
VPAGTIIGDESRSVTLSSSSEEEKNDLGEVQTSPTEKNDPGEVQTSSTDHLNNSQSEESSEVSPETSSVSNSTTATSLEKSPNPTASIVYGQVHLNQLLNTLLPHRRSLNNSNPTDRSP

SEQ ID NO: 244 - protein, 143 aa, Nostoc sp PCC 7120
VPPGSILGDTTRQVAATQSPSTSKNQVGETTQKPKENESKVITSTTLSASAFVEFKQHSVSVTEPPPSSENQSATVEENTTNGTDPNVTELSPEDSASDQPATESPNSFGTQIYGQGSIQRLLVTLFPHRQALNNPVSDDSSE

SEQ ID NO: 245 - protein, 143 aa, Anabaena variabilis
VPPGSILGDTTRQLAATESPATSTNQVDEATQKPKENESKVITSTTLSASAFVEFKQHSVSVTEPPPSPENQSATVEENTTNGTDPNVTELSPEDSASDQSATESPNSFGTQIYGQGSIQRLLVTLFPHRQALNNPVSDDSSE

SEQ ID NO: 246 - protein, 160 aa, Nodularia spumigena
IPPGSILGDTSRQIEDTEQLESSTNNGDHTSTEQQPEAENSLETDEETVISSTTISAKAYWKFKHQSTSSSGSSPTSSSQPAPVEPAPVEPAPVEPAPVEQKAKASNSIPQKSKSSQPPTESPNSFGNQIYGQVSINRLLVTLFPHRQTLNDSISDDQSE

SEQ ID NO: 247 - protein, 152 aa, Nostoc punctiforme
VPPGSILGDTSRQIAQTTQPEPSTNNSTATSVPPQKEEENGSGGVKEKVSSSTNFSAAAFVDFKQNKSISYFKSPATPESQPPPLEEPAKDAESPLQEAVQEPTKSDSDPNQLPTESPNGFGTQIYGQGSISRLLTTLFPHRQSLSDPNSDD

SEQ ID NO: 248 - protein, 126 aa, Cyanothece sp PCC 7425
VAPGSLVGDPSRRPPELTETEALQEEQPTHLQPAQSQSDEPQTDQSPAAQEEQGDLQSASPAPVDHAAGTNSSPSPQAEQQTDAPPRSVYGQDYVNRMMQRMMPRTPSLTPSPTGQNGSVEGGTGS

SEQ ID NO: 249 - protein, 115 aa, Thermosynechococcus elongatus
VAAGSLVGDRSRRWPPAAETSHPQQRTVFPEDPWQEPATTAHTSENSPQQEQEATDSPPNHQESPAAAPPETSTATRPKASVVYGQAYVSKMFAKMFRVAPIPPTGDNSALGSSQ

SEQ ID NO: 250 - protein, 56 aa, Synechococcus elongatus PCC 6301
TAPGSLLSAETPPTTATVSSSEPAGRSPQSSAIAHPTKVYGKEQFLRMRQSMFPDR

SEQ ID NO: 251 - protein, 56 aa, Synechococcus elongatus PCC 7942; identical to SEQ ID NO: 250

SEQ ID NO: 252 - protein, 25 aa, Acaryochloris marina MBIC11017
SHVIGRAYVTQMLQVLFARNSSPYP

SEQ ID NO: 253 - protein, 32 aa, Trichodesmium erythraeum
DAQIYGKEYVNKIMQTLFPYKNSLSSHPDDED

SEQ ID NO: 254 - protein, 20 aa, Synechococcus elongatus PCC 6301
TKVYGKEQFLRMRQSMFPDR

SEQ ID NO: 255 - protein, 20 aa, Synechococcus elongatus PCC 7942
TKVYGKEQFLRMRQSMFPDR

SEQ ID NO: 256 - protein, 34 aa, Gloeobacter violaceus
PPVAGKAYLERLRLSLFPHNAPLQNPDSATGGGA

SEQ ID NO: 257 - protein, 26 aa, Synechococcus sp. JA-3-3Ab
GYSELDRLLGKIYPYRQILSSGGGQS

SEQ ID NO: 258 - protein, 30 aa, Synechococcus sp. JA-2-3B′a(2-13)
NGIPGHWELDRLLSKIYPHRQVLSSGDSRL

SEQ ID NO: 259 - protein, 34 aa, Nodularia spumigena
GNQIYGQVSINRLLVTLFPHRQTLNDSISDDQSE

SEQ ID NO: 260 - protein, 31 aa, Nostoc punctiforme
GTQIYGQGSISRLLTTLFPHRQSLSDPNSDD

SEQ ID NO: 261 - protein, 34 aa, Anabaena variabilis
GTQIYGQGSIQRLLVTLFPHRQALNNPVSDDSSE

SEQ ID NO: 262 - protein, 34 aa, Nostoc sp PCC 7120; identical to SEQ ID NO: 261

SEQ ID NO: 263 - protein, 33 aa, Lyngbya sp PCC 8106
SIVYGQVHLNQLLNTLLPHRRSLNNSNPTDRSP

SEQ ID NO: 264 - protein, 32 aa, Synechococcus sp PCC7002
TPVVGQVYINQLLMTLFPHQNSLNTPNQPDEP

SEQ ID NO: 265 - protein, 31 aa, Microcystis aeruginosa
ASVVGKVYINQLLVTLFPERHRFNGNNNHNS

SEQ ID NO: 266 - protein, 32 aa, Cyanothece sp PCC8801
APVVGQIYINQLLYTLFPERQAFNRSQNGSSS

SEQ ID NO: 267 - protein, 32 aa, Cyanothece sp PCC8802; identical to SEQ ID NO: 266

SEQ ID NO: 268 - protein, 38 aa, Cyanothece sp CCY0110
APVVGQVYINQLLCTLFPDRQAFNQAQNNPPSQDENNE

SEQ ID NO: 269 - protein, 40 aa, Cyanothece sp ATCC51142
APVVGQVYINQLLCTLFPDRQAFNQSQNHSASDNSANNNK

SEQ ID NO: 270 - protein, 40 aa, Crocosphaera watsonii
APVVGQVYINQLLCTLFPDRQAFNQSQNNSASKDPPGKNK

SEQ ID NO: 271 - protein, 25 aa, Synechocystis sp PCC 6803
PPVVGQVYINQLLLTLFPERRYFSS

SEQ ID NO: 272 - protein, 40 aa, Cyanothece sp PCC 7822
APVVGQIYINQLLLTLFPERRFFQNLDQKNQSLHSEENSQ

SEQ ID NO: 273 - protein, 35 aa, Thermosynechococcus elongatus
SVVYGQAYVSKMFAKMFRVAPIPPTGDNSALGSSQ

SEQ ID NO: 274 - protein, 40 aa, Cyanothece sp PCC 7425
RSVYGQDYVNRMMQRMMPRTPSLTPSPTGQNGSVEGGTGS

SEQ ID NO: 275 - protein, 76 aa, Lactobacillus brevis
MAQEIDENLLRNIIRDVIAETQTGDTPISFKADAPAASSATTATAAPVNGDGPEPEKPVDWFKHVGVAKPGYSRDE

SEQ ID NO: 276 - protein, 78 aa, Desulfatibacillum alkenivorans
MKLTEEMLRQIITEVVGQMAGGAAAPAPAAVDTDKPLNFIEKGPAQAGSNPKEVVVAVPPGFGVTPTKTIIDIPHSVV

SEQ ID NO: 277 - protein, 78 aa, Sebaldella termitidis
MNIDEKQLKDIIAGVIKEIQNEKGNCGCTSDGKISFGQGSSDNRLKLNENGQAKQGTRSDEVVIGIAPAFGESQTETI

SEQ ID NO: 278 - protein, 77 aa, Thermoanaerobacter sp. X514
MVKTESLVEQIVKEVLKKLENVEIAAPATQSSDDANQEWEMIIEEIGEAKQGVNVDEVVIGVSPGFYIKFKKNIIGI

SEQ ID NO: 279 - protein, 78 aa, Thermosediminibacter oceani
MINTEMVVEEVVKEVLKRLAGEREKVAEDYAVGNPAGKELLLEEMGEAKPGAREEEVVIGVSPAFGVKFKENINGIPL

SEQ ID NO: 280 - protein, 78 aa, Dethiosulfovibrio peptidovorans
MINEELVRKVIAEVLQEVAASENVESASVTARPSAPAVKAEISMEMTEKERATRGTDAREVVVAIPPAFGTEFDATIV

SEQ ID NO: 281 - protein, 77 aa, Yersinia bercovieri
MVDINEKLLRQIIEGVLQEMQGEKNSVSFKQESQPATAVASGDFLTEVGEARPGSNQDEVIIAVGPAFGLSQTANIV

SEQ ID NO: 282 - protein, 77 aa, Klebsiella pneumoniae
MEINETLLRQIIEEVLSEMKSGADKPVSFSASAASVASAAPVAVAPVSGDSFLTEIGEAKPGTQQDEVIIAVGPAFG

SEQ ID NO: 283 - protein, 77 aa, Shigella sonnei
MEINEKLLRQIIEDVLAEMQPSDKSVSFRAPVSATVPSAPDTGNFLTEIGEAQQGTQQDEVIIAVGPAFGLAQTVNI

SEQ ID NO: 284 - protein, 77 aa, Escherichia coli
MEINEKLLRQIIEDVLAEMQPSDKSVSFRAPVSATVSSAPDTGNFLTEIGEAQQGTQQDEVIIAVGPAFGLAQTVNI

SEQ ID NO: 285 - protein, 77 aa, Citrobacter koseri
MEINEKLLRQIIEDVLSEMQTSDKPVSFRAPTASTSPQAAAPQDDGFLTEIGEARQGTQQDEVIIAVGPAFGLSQTV

SEQ ID NO: 286 - protein, 77 aa, Salmonella typhimurium
MEINEKLLRQIIEDVLRDMKGSDKPVSFNAPAASTAPQTAAPAGDGFLTEVGEARQGTQQDEVIIAVGPAFGLAQTV

SEQ ID NO: 287 - protein, 77 aa, Salmonella enterica
MEINEKLLRQIIEDVLRDMKGSDKPVSFNTPAASTAPQTAAPAGDGFLTEVGEARQGTQQDEVIIAVGPAFGLAQTV

SEQ ID NO: 288 - protein, 67 aa, Lactobacillus brevis
MSEIDDLVAKIVQQIGGTEAADQTTATPTSTATQTQHAALSKQDYPLYSKHPELVHSPSGKALNDIT

SEQ ID NO: 289 - protein, 58 aa, Sebaldella termitidis
MDEVMIKNMVKEILNNIEKHDSGKKDSSGKIGVSSYPLGSRRPDLVRTPTNKTLDDIT

SEQ ID NO: 290 - protein, 68 aa, Dethiosulfovibrio peptidovorans
VEINEKLIAEMVRQVLQSGGNQEKGASNSPQETSVKDRKVLSKNDYPLAVKRPELLVGPRGKGFDELT

SEQ ID NO: 291 - protein, 66 aa, Thermoanaerobacter sp. X514
MIDEKTLEIIVREVLTNLTSDKGTQNQQKTASSSLPKLDPKRDYPLAKNKPELAKSITGKTINEIT

SEQ ID NO: 292 - protein, 63 aa, Thermosediminibacter oceani
MIDEKALEEIVRQVLEELGSHKKQVKAEIKKDEGLDPKLDFPLSKKRPELLKSATGKKFTEIT

SEQ ID NO: 293 - protein, 66 aa, Yersinia bercovieri
MNSEAIESMVRDVLNKMNSLQGQAPAACPAPAASSRSDAKVSDYPLANKHPDWVKTATNKTLDDLT

SEQ ID NO: 294 - protein, 66 aa, Klebsiella pneumoniae
MNTDAIESMVRDVLSRMNSLQDGITPAPAAPTNDTVRQPKVSDYPLATRHPEWVKTATNKTLDDLT

SEQ ID NO: 295 - protein, 64 aa, Shigella sonnei
MNTDAIESMVRDVLNRMNSLQDAAPVSAVPNASILSAKVTDYPLANKHPEWVKTATNKTLDDFT

SEQ ID NO: 296 - protein, 64 aa, Escherichia coli; identical to SEQ ID NO: 295

SEQ ID NO: 297 - protein, 65 aa, Salmonella enterica
MNTDAIESMVRDVLSRMNSLQGDAPAAAPAAGGTSRSAKVSDYPLANKHPEWVKTATNKTLDDFT

SEQ ID NO: 298 - protein, 65 aa, Salmonella typhimurium; identical to SEQ ID NO: 297

SEQ ID NO: 299 - protein, 64 aa, Citrobacter koseri
MNTDAIESMVRDVLSRMNSLQGNAPAPAAASASTHTAKVTDYPLANKHPEWVKTATNKTLDEFT

SEQ ID NO: 300 - protein, 103 aa, Bacillus sp. B14905
VNDQLVSMITQLVMEKMEKTTEGQAPEVITTRTEEPLIKFYDTAATKGATELAKPMSTTSEPLIQLYQQGTPQQAHIAPATFEQPLNVAVPIKPFQFEADTLT

SEQ ID NO: 301 - protein, 103 aa, Nocardioides  sp. JS614
MSTDELRSIVAEVLAELAEPGDAFARLTTPATTAGPSGPTSTPAPEESDAPSSAATEPAAVPASSATEITRPTLSGAPVSIEVSDPTVPEARHRIGVENPANP

SEQ ID NO: 302 - protein, 103 aa, Alkaliphilus metalliredigens QYMF
ISEQAVKEMVQQIVEQMTIGQKQTTEDKYTQETDGKEQPEICIEDKNLKDLTEIKMQDYFAVPNPENKEVYLGLKEQTPARVGIWRTGSRNSTETLLRFRADH

SEQ ID NO: 303 - protein, 103 aa, Leptotrichia buccalis C-1013-b
LSERELKDVIEKIISEIKIEETPAKETPVTVMEEKTPVVSTSSTYDQDENPRENPHIVNGEVRDIGKINVKEQMLVDNPEDREEYMKLKQKTSARLGIGRAGT

SEQ ID NO: 304 - protein, 103 aa, Sebaldella termitidis ATCC 33386
LSERELREIIGKVIDEMGSNGKTDIPAAVGNDFKASSSVKENVSDDQLVDLGEINIKDQLLVDNPANREEYMKLKQRTSARLGIGRAGTRFKTDVLLRFRADH

SEQ ID NO: 305 - protein, 103 aa, Fusobacterium nucleatum ATCC 25586
VSELELKEIIGKVLKEMAVEGKTEGQAVTETKKTSESHIEDGIIDDITKEDLREIVELKNATNKEEFLKYKRKTPARLGISRAGSRYTTHTMLRLRADHAAAQ

SEQ ID NO: 306 - protein, 103 aa, Bacteroides capillosus ATCC 29799
MNEKDLRSIIEQVLAEMNGAGEAKEAAPSCCTAAPVEESCKVEEGCLPDITEIDIREQYLVKDPENGEEYAELKMNAPCRLGIGKAGARYNTLPQLEFRAAHS

SEQ ID NO: 307 - protein, 103 aa, Clostridium phytofermentans ISDg
MDEQSLRKMVEQMVEQMVGGGTNVKSTTSTSSVGQGSATAISSECLPDITKIDIKSWFLLDHAKNKEEYLHMKSKTPARLGVGRAGARYKTMTMLRVRADHAA

SEQ ID NO: 308 - protein, 103 aa, Streptococcus sanguinis SK36
MDELQLKEMIRSLLNEMGGDSAVKETAATDQNKAEKPAVSLQEEVKQDTSVIEDGIIPDITEVDIQEQFLVPNAINEEAYRKIKKFTPARLGLWRAGDRYKTQ

SEQ ID NO: 309 - protein, 103 aa, Thermanaerovibrio acidaminovorans Su883
VKEQDLKQLVMEILNEMSRGAEPSPTQPSTPPQGAQEAPSGQEGELPDLTQVDIRTQCLVPSPKDPAALMAMKAKTPARIGVWRAGPRYKTETLLRFRADHAA

SEQ ID NO: 310 - protein, 103 aa, Enterococcus faecalis V583
MNEKELKEMIAGILTEMVADNQAVSTATVTAEEKPVTTHVTETTEIEEGLIPDITEVDLRKQLLLKNAVDPEALLKMKAFSPARLGVGRAGTRYMTSSTLRFR

SEQ ID NO: 311 - protein, 103 aa, Alkaliphilus oremlandii OhILAs
MDELNLKEMIKSILNEMVGEAPPAVINSNSTAERSVGTMQTTKPQGVEERFIPDITAVDIRKQFLVPNAADKEGYLKMKSYTPARLGLWRAGPRYMTEPSLRF

SEQ ID NO: 312 - protein, 103 aa, Clostridium difficile 630
MNEKDLKALVEQLVGQMVGELDTNVVSETVKKATEVVVDNNACIDDITEVDIRKQLLVKNPKDAEAYLDMKAKTPARLGIGRAGTRYKTETVLRFRADHAAAQ

SEQ ID NO: 313 - protein, 103 aa, Listeria monocytogenes 10403S
MNEQELKQMIEGILTEMSGGKTTDTVAAVPTKSVVETVVTEGSIPDITEVDIKKQLLVPEPADREGYLKMKQMTPARLGLWRAGPRYKTETILRFRADHAVAQ

SEQ ID NO: 314 - protein, 103 aa, Marinobacter aquaeolei VT8
MDEQTIQSIVNSVLRELGEKDLPAGQVTRVQPEGKSTQRNDPPAYKPSETAGRQGQTESADTGDGLEDLSLEKFVHWNGIENAHNASVNSDMVKQTAARVCQG

SEQ ID NO: 315 - protein, 102 aa, Yersinia intermedia ATCC 29909
MDQKQIEEIVRSVMLRMGQVEVATQPASAAASADTVECCSMDLGSEEAKQWIGVTNPQRLDVLQELRSSTAARVCTGRAGPRPRTQALLRFLADHSRSKDTV

SEQ ID NO: 316 - protein, 103 aa, Klebsiella pneumoniae
MDQKQIEDIVRSVMASMGQPQSQPQAPAASTPACHAACASEAVVESCALDLGSAEAKAWIGVQHPHRAEVLTELKRSTAARVCTGRAGPRPRTQALLRFLADH

SEQ ID NO: 317 - protein, 103 aa, Citrobacter koseri
MDQKQIEEIVRSVMASMGESQPQAPAESAPACSAKQCAAPSAPSAAESCALDLGSAEAKAWVGVENPHRADVLAELRRSTAARVCTGRAGPRPRTLALLRFLA

SEQ ID NO: 318 - protein, 103 aa, Escherichia coli HS
MDQKQIEEIVRSVMASMGQTAPAPSEAKCATTNCAAPVTSESCALDLGSAEAKAWIGVENPHRADVLTELRRSTVARVCTGRAGPRPRTQALLRFLADHSRSK

SEQ ID NO: 319 - protein, 103 aa, Salmonella Typhimurium LT2
MDQKQIEEIVRSVMASMGQDVPQPAAPSTQEGAKPQCAAPTVTESCALDLGSAEAKAWIGVENPHRADVLTELRRSTAARVCTGRAGPRPRTQALLRFLADHS

SEQ ID NO: 320 - protein, 100 aa, Salmonella enterica Paratyphi A ATCC 9150
MDQKQIEEIVRSVMASMGQDVPQPVAPSKQEGAKPQCASPTVTESCALDLGSAEAKAWIGVENPHRADVLTELRRSTAARVCTGRAGPRPRTQALLRFLA

SEQ ID NO: 321 - protein, 103 aa, Photobacterium profundum 3TCK
MNEQKIQDIVATVLAQLGETNVAASDITKVVNAVTPAAGGYVPQVSAESLPDLGDIQFKKWNGIQNAVDKKVVEDLMSQTDARVGTGRTGPRPRTTALLRFLA

SEQ ID NO: 322 - protein, 103 aa, Shewanella benthica KT99
MNEQNIKNIVATVLAQLGENNIQPSTITKVIDAASNVAGKTVISDESLPDLGEPRFKKWNGVINAANPSIVDDLMSQTNARMGTGRTGPRPRTIPLLRFLADH

SEQ ID NO: 323 - protein, 106 aa, ANHYDRO_00930
ISLEELKEALENNFGFTDSIMPGPCGGDSVSAKVGQLSEAEIYDAIKKILSNSDTTDVDEIAKKLELNNTENSSYQSACGCSANETGRFKTIQKILDNTGSFGNDD

SEQ ID NO: 324 - protein, 104 aa, PepasDRAFT_0461
ISLADLKEALEKNFGFTDSLMPGCGCNTQTVSAKVGEMNESEIYEAVKKILASTGSINVDDLENKLNEEYVVSGDCGCGSQETTGKFRTIQKILDNTDSFGNDN

SEQ ID NO: 325 - protein, 96 aa, c4537
LSLSELKSALDANFGYPVGANPHTPAAKSSLNEQDIYDVVKRIIEQHGALDPAAIKNEVYRQLTSGSAAPVQSGTMSRHEEIRRILENTPCFGNDI

SEQ ID NO: 326 - protein, 96 aa, AECO1_2293; identical to SEQ ID NO: 325

SEQ ID NO: 327 - protein, 96 aa, ecoli_01002098; identical to SEQ ID NO: 325

SEQ ID NO: 328 - protein, 93 aa, rru_A0903
ITLAEMKEALDANFGLPVGGSAPSAGGDFTEEQVFAAVRKVLSSNGSMDVSALKGEVYRTLSGQAAPAAGGSSTKYDAIRRLLDASPAFGNDI

SEQ ID NO: 329 - protein, 94 aa, rpc_1163
ITLGELKAALDANFGRPVGESAHADAGTNYTEEQVFAAVKKVLNSSGSTDVSALKGKVYSALAGANGAKSGGASSSYDALHRLLEATPAFGNDI

SEQ ID NO: 330 - protein, 91 aa, cbei_4061
IEMDQLKAALDANFGHTGVNTVSTSNNNADVTEMQIYEAVKRILSNSGSIDISEIQSRISSEFTSPKTTVSGDFDNIRRLLESTPCFGNDI

SEQ ID NO: 331 - protein, 103 aa, clobol_08236
VTMAQLKEAMANNFGYACNASAPAATADECTDEARIYEAVKRILSNNGSINLADLQAQLAGPAQACRWPSPAEPAKTEPACVNPDYAHIKRLMENTPWFGNDI

SEQ ID NO: 332 - protein, 90 aa, NT01CX_0498
VSMGDLKEALDTNFGECNSSNSLNLNSINNINPENLNRETIMAVIEKLLFKESNISVNNLNSNINLGNYQGKESLRQMLINRAPKYGNDI

SEQ ID NO: 333 - protein, 93 aa, sputw3181_0427
LSLGHLKEALDANFGVSGGIEKPDTIATESTPKQDATYELVLEAVKKVLGESGALALTSLNSNPPEPVKGANAGLTAVRQLLINGAPKFGNDI

SEQ ID NO: 334 - protein, 93 aa, SPUTCN32_0208; identical to SEQ ID NO: 333

SEQ ID NO: 335 - protein, 147 aa, CLOSTASPAR_02209
EYYATILMYTGNIIGQQNHLSCEQVDRLLEIRKNMGITGGGVPPCMNGGQLTKVCESCAAAGEKTAAAGTELAGGSCGGCAAAGGTQTGPQAPLKGVTPLVRPGDAGKMPGGGLGAGSGSPSTGSGPADKDALIAEIVRRVVVQLKA

SEQ ID NO: 336 - protein, 78 aa, BselDRAFT_1650
EHYALMTMYSTNIIQKTNELNCDQISDLMGIRSKLGIHSGGTPSCQPERQETKKDVDIEAIVAAVTQEVIGKLQERRN

SEQ ID NO: 337 - protein, 99 aa, ANACOL_01089
EYYALVTMYTGSIIGQANELSCEQIDQLVDTRTRLGISTGGRPVCQNVGKDGVPACMEQKKCGGQCTHGGQPPAGADAGTVAMEDIVDIVRQVMARTKR

SEQ ID NO: 338 - protein, 107 aa, CLOSTMETH_00022
EYFAKVSMYCRQLGGAQQLDCSQINRLLELREEFKAPGKHPGCPQCQVLPAEAVPVNTANPDGTQRRQPAAVIPGEIPAGVAPAAAAPSDNDLIAEITRKVLAQLGK

SEQ ID NO: 339 - protein, 100 aa, GCWU000342_00652
EFYAQLLYQSKMLGGPKEFDQKTVERLYEIRRQMGLPGKHPANLCQNKDGHNCHNCGLHQEIPGMPASGATTGSITSTPKEPAPEVIAEITKRVLEQLGK

SEQ ID NO: 340 - protein, 80 aa, ROSEINA2194_01705
EFYAELLYKAKQLGGPKEFDKEQIAKLYEIRRKMGLPGRHPANLCQNKGKENCHNCGGGCSSSAQVDDNKELVAAITKKY

SEQ ID NO: 341 - protein, 110 aa, RUMOBE_00095
EFYAQLLYQSKLLGGPKEFDKENIKKLYEIRRKFGMPGKHPANLCQNKDGVNCHNCGGACHSQDYKQFPGYQYDFVGSETKAEAPAATGAADAELVANITKQVMAQLGMK

SEQ ID NO: 342 - protein, 86 aa, Cphy_1177
EFYAQLLYQAKVLGGPKELSNSQVQRLYELRREFGLKGKHPANLCSNTKEGKASCHCCGEECKSGGVDNADLVASITRKVMEQLGL

SEQ ID NO: 343 - protein, 90 aa, RUMGNA_01020
EFYARLLWQTMQIGGPQELNKEQVEKLYEIRRQMGLSGKHPANLCPNAKAGKPSCHSCGGGCGAAKTEETPDADLVASITKKVMDQLGLN

SEQ ID NO: 344 - protein, 148 aa, IsopDRAFT_2610
DAYCRILILARQLGRVQYYPDEKAAELIRLKPNLGIRDVRLELGLENCDLCGNSLFREGYSDFKPEPYAFRHPRLGGDATGIGPVAGPHSTNANANVNANASPPIQVQPGSPEFEQMVQMITDEIMGHLAGRSTSVSASAAASNPGGC

SEQ ID NO: 345 - protein, 111 aa, PM8797T_14741
DAYCNILLLSKQLGRVTYFTENETRELLDLKKKLGFDDPRFHVEDCDLCGNSAFRDGYKEGIPQQKSFEPAPSYPGYLSKPSTQATPATNNGDSDQLIKAITDQVMSALGK

SEQ ID NO: 346 - protein, 117 aa, Plim_1747
DAYCRILLLSKQLGRVEYLNERESVELLDLKKKLGFDDPRFHVENCDLCGNSAFREGYKDAQPQPAAFEPAPYYPGYLERQKSTPAPAAAPSAAAAPVDTEMLVKMITEQVMAALKK

SEQ ID NO: 347 - protein, 110 aa, RB2568
DSYCRMLLLAKQLGNVSYLDETKSRELLELKDKWGFKDPRNTSEYEDCDICANDIFRDSWKDSGVERRAFAPPPPIKTSGSASSAPAGVDEEQLVKLITNEVMRQMKASS

SEQ ID NO: 348 - protein, 110 aa, DSM3645_04920
DAYCRMLILAKQLGRVEFFSEEKERELLDLKQRWGWSDPRNTEEYKDCDICANDIFRDSWKDSLIERKAFPAPPAMGPNANKAAAPVTGDQEALIQAITSRVMAELSKRS

SEQ ID NO: 349 - protein, 110 aa, Psta_3288
DAYCRMLMLAKDLGRVNYFSEKKERELLELKDKWGWKDPRNTPEYKDCDICANDIFRDSWKQSGVERKAFEAPPPMAPSAKKEAAPAAAGDQEALVRLITERVLAELSKK

(19) United States
(12) Patent Application Publication
(10) Pub. No.: US 2016/0222068 A1
(43) Pub. Date: Aug. 4, 2016

(54) CONSTRUCTS FOR EXPRESSING BIOLOGICAL MOLECULES THAT INTEGRATE INTO BACTERIAL MICROCOMPARTMENTS

(71) Applicant: The Regents of the University of California, Oakland, CA (US)

(72) Inventors: Cheryl A. Kerfeld, Walnut Creek, CA (US); James N. Kinney, Clayton, CA (US)

(21) Appl. No.: 15/134,259

(22) Filed: Apr. 20, 2016

Related U.S. Application Data
(62) Division of application No. 13/564,676, filed on Aug. 1, 2012.
Application No. 13/564,676 is a continuation of application No. PCT/US2011/023416, filed on Feb. 1, 2011.
(60) Provisional application No. 61/300,338, filed on Feb. 1, 2010.

(51) Int. Cl. C07K 14/195; C12N 9/00

(52) U.S. Cl. CPC C07K 14/195; C12N 9/00

(57) ABSTRACT

A conserved region of sequence in bacterial microcompartment (BMC) enzymes and proteins was identified. Peptide sequences derived from this conserved region of native BMC proteins and enzymes appear to target the hexameric facets of BMC shell proteins. These peptides were predicted to share general properties of a predicted alpha helical conformation, flanked by poorly conserved segment(s) of primary structure); for each type of encapsulated protein, and for each functionally distinct BMC. These peptides can be used as targeting signals for integrating biomolecules and molecules into bacterial microcompartments or for attaching molecules or biomolecules to native or non-native bacterial microcompartment shell proteins.

### CROSS-REFERENCE TO RELATED APPLICATIONS

This application is divisional application of U.S. patent application Ser. No. 13/564,676, which is a continuation-in-part application of International Patent Application No. PCT/US2011/023416, filed on Feb. 1, 2011, which claims priority to U.S. Provisional Application No. 61/300,338, filed on Feb. 1, 2010, all of which are hereby incorporated by reference in their entirety. This application is related to and incorporates by reference U.S. patent application Ser. No. 13/367,260, filed on Feb. 6, 2012 in its entirety for all purposes.

### STATEMENT OF GOVERNMENTAL SUPPORT

This invention was made with government support under Contract No. DE-AC02-05CH11231 awarded by the U.S. Department of Energy. The government has certain rights in the invention.

### REFERENCE TO SEQUENCE LISTING AND TABLES

This application also incorporates by reference the attached sequence listings which is also found in computer-readable form in a *.txt file entitled, “2785US_sequencelisting_asfiled_ST25.txt”, created on Apr. 20, 2016.

### BACKGROUND OF THE INVENTION

1. Field of the Invention

The present invention relates to synthetic biology, especially using targeting signals for integrating biomolecules and molecules into bacterial microcompartments or for attaching molecules or biomolecules to bacterial microcompartment shell proteins.

2. Related Art

Bacterial microcompartments (BMCs) encapsulate functionally related reactions. BMC shell proteins and the components they encapsulate are typically found in gene clusters (putative operons). The shells of BMCs are generally comprised of multiple paralogs of proteins containing the BMC domain (e.g., Pfam 00936) and presumably a relatively small number of proteins containing the Pfam03319 domain. There is recognizable sequence homology among the >2000 BMC domain-containing proteins now in the sequence databases, suggesting that despite functional diversity and some differences in the morphology of a specific BMC type, there are conserved structural determinants for targeting and binding of the enzymes and auxiliary proteins that are encapsulated in BMCs.

Carboxysomes are the foremost example of the polyhedral subcellular inclusions that have been termed bacterial microcompartments, self-assembling protein shells that encapsulate enzymes and other functionally related proteins. In addition to carboxysomes, two other types of bacterial microcompartments (BMCs) are relatively well characterized by others; they function in propane-diol utilization (encoded by the pdu operon) and ethanolamine utilization (encoded by the eut operon) in heterotrophic bacteria. Carboxysomes have been observed in all cyanobacteria and in many chemoautotrophs.

### BRIEF SUMMARY OF THE INVENTION

The present invention describes a common motif (peptide) found in a subset of proteins presumed to be encapsulated in functionally diverse bacterial microcompartments (BMCs). This common motif and adjacent linker region were identified as important for targeting proteins to BMCs. All BMC targeting peptides share general properties such as a region predicted to have an alpha helical conformation, adjacent to poorly conserved segment(s) of primary structure enriched in proline and glycine; for each type of encapsulated protein, for each functionally distinct BMC. Amino acid properties are conserved in many of the positions within these peptides. We have also identified a consensus amino acid sequence for the targeting peptide specific to various BMC types.

The present invention also provides for an isolated polypeptide comprising a sequence selected from SEQ ID NOS: 1-349 or a fragment thereof. An expression cassette comprising a polynucleotide encoding a peptide selected from SEQ ID NOS: 1-349 or a fragment thereof can be made. The expression cassette further comprising a cluster of microcompartment genes isolated from a bacteria, wherein the cluster comprising a set of microcompartment genes necessary for the expression of a microcompartment.

The expression cassette can be used to provide a cell comprising in its genome at least one stably incorporated expression cassette, where the expression cassette comprising a heterologous nucleotide sequence of any of SEQ ID NOS: 1-349 or a fragment thereof operably linked to a promoter that drives expression in the cell.

Also provided are methods for enhancing metabolic activity in an organism. In one method, comprising introducing into an organism at least one expression cassette operably linked to a promoter that drives expression in the organism, where the expression cassette comprising a cluster of microcompartment genes isolated from a bacteria, wherein the cluster comprising a set microcompartment genes necessary for the expression of a microcompartment that has metabolic, wherein the microcompartment genes further comprise a polynucleotide expressing a peptide of SEQ ID NOS: 1-349 or a fragment thereof.

### BRIEF DESCRIPTION OF THE SEQUENCES

SEQ ID NOS: 1-22 are actual localization peptide sequences from proxy organisms and shown in Table 2.

SEQ ID NOS: 23-44 are consensus peptide sequences for specific BMC-associated pathway enzymes and proteins as shown in Table 3.

SEQ ID NO: 45 is the consensus peptide motif as described in FIG. 3C.

SEQ ID NO: 46 is a consensus peptide sequence derived from the conserved C-termini in carboxysomal protein, CcmN, in Synechocystis sp. PCC6803 and Synechococcus elongatus PCC7942.

SEQ ID NOS: 47-69 are peptide sequences obtained from GenBank for organisms listed in FIGS. 1, 2a, and 2b.

SEQ ID NOS: 70-82 are peptide sequences obtained from GenBank for organisms listed in FIG. 5a.

SEQ ID NOS: 83-94 are peptide sequences obtained from GenBank for organisms listed in FIG. 6.

SEQ ID NOS: 95-117 are peptide sequences obtained from GenBank for organisms listed in FIG. 8.

SEQ ID NOS: 118-129 are peptide sequences obtained from GenBank for organisms listed in FIG. 10.

SEQ ID NOS: 130-144 are peptide sequences obtained from GenBank for organisms listed in FIG. 11a.

SEQ ID NOS: 145-190 peptide sequences used for helical wheel projection of the predicted alpha helix of various regions of CcmN in various organisms from FIGS. 4a, 4b, 5b, 5c, 7a, 7b, 9a 9b 12-24.

SEQ ID NOS: 191-193 are various parts of the CcmN protein sequences used in transformation in Examples 2 and 3.

SEQ ID NOS: 194-205 are peptide sequences of the N-terminal region of a pdu-associated aldehyde dehydrogenase (PduP) from 12 microorganisms from FIG. 3a.

SEQ ID NOS: 206-228 are sequences for CcmN protein of various cyanobacteria from FIG. 1.

SEQ ID NOS: 229-251 are peptide sequences of the conserved N-terminal domain and variable regions of the CcmN protein of various organisms from FIG. 2a.

SEQ ID NOS: 252-274 are peptide sequences for the targeting peptide region of the CcmN protein of various organisms from FIG. 2b.

SEQ ID NOS: 275-287 FIG. 5a are peptide sequences for the N-terminal region of Diol dehydratase medium subunit (PduD) from 13 microorganisms from FIG. 5a.

SEQ ID NOS: 288-299 are peptide sequences for the N-terminal region of diol dehydratase small subunit (PduE) from 11 microorganisms from FIG. 6.

SEQ ID NOS: 300-322 are peptide sequences for the N-terminal region of the EutC (Ammonia lyase light chain) N-terminal region from 23 microorganisms from FIG. 8.

SEQ ID NOS: 323-334 are peptide sequences for B12-independent diol dehydratase interdomain peptide of various organisms from FIG. 10.

SEQ ID NOS: 335-349 are peptide sequences for L-Fuculose phosphate aldolase C-terminal region of various organisms from FIG. 11a

### BRIEF DESCRIPTION OF THE DRAWINGS AND TABLES

FIG. 1 is an alignment of the primary structure of CcmN, a protein encapsulated in the carboxysome, from various cyanobacteria with secondary structure prediction. SEQ ID NO: 206 is Synechococcus_sp._JA-3-3Ab, SEQ ID NO: 207 is Synechococcus_sp._JA-2-3B′a(2-13), SEQ ID NO: 208 is Trichodesmium_erythraeum, SEQ ID NO: 209 is Synechococcus_sp_PCC7002, SEQ ID NO: 210 is Cyanothece_sp_PCC8801, SEQ ID NO: 211 is Cyanothece_sp_PCC8802, SEQ ID NO: 212 is Crocosphaera_watsonii, SEQ ID NO: 213 is Cyanothece_sp_CCY0110, SEQ ID NO: 214 is Cyanothece_sp_ATCC51142, SEQ ID NO: 215 is Acaryochloris_marina_MBIC11017, SEQ ID NO: 216 is Cynotece_sp_PCC7822, SEQ ID NO: 217 is Microcystis_aeruginosa, SEQ ID NO: 218 is Synechocytis_sp_PCC6803, SEQ ID NO: 219 is Gloeobacter_violaceus, SEQ ID NO: 220 is Lyngbya_sp_PCC8106, SEQ ID NO: 221 is Nostoc_sp._PCC7120, SEQ ID NO: 222 is Anabaena_variabilis, SEQ ID NO: 223 is Nodularia_spumigena, SEQ ID NO: 224 is Nostoc_punctiforme, SEQ ID NO: 225 is Cyanothece_sp_PCC7425, SEQ ID NO: 226 is Thermosynechococcus_elongatus, SEQ ID NO: 227 is Synechococcus_elongatus_PCC6301 and SEQ ID NO: 228 is Synechococcus_elongatus_PCC7942.

FIGS. 2a and 2b are close-ups of the alignment and secondary structure prediction of the C-terminal region of the CcmN protein in various organisms. FIG. 2A shows the CcmN, C-terminal alignment and secondary structure predictions of the conserved N-terminal domain and variable regions of various organisms. SEQ ID NO: 229 is Synechococcus_sp._JA-3-3Ab, SEQ ID NO: 230 is Synechococcus_sp._JA-2-3B′a(2-13), SEQ ID NO: 231 is Trichodesmium_erythraeum_IMS101, SEQ ID NO: 232 is Synechococcus_sp. 7002, SEQ ID NO: 233 is Cyanothece_sp._PCC8801, SEQ ID NO: 234 is Cyanothece_sp._PCC8802, SEQ ID NO: 235 is Crocosphaera_watsonii_WH8501, SEQ ID NO: 236 is Cyanothece_sp._CCY0110, SEQ ID NO: 237 is Cyanothece_sp._ATCC51142, SEQ ID NO: 238 is Acaryochloris_marina_MBIC11017, SEQ ID NO: 239 is Cynotece_sp._PCC7822, SEQ ID NO: 240 is Microcystis_aeruginosa, SEQ ID NO: 241 is Synechocytis_sp._PCC6803, SEQ ID NO: 242 is Gloeobacter_violaceus, SEQ ID NO: 243 is Lyngbya_sp._PCC8106, SEQ ID NO: 244 is Nostoc_sp._PCC7120, SEQ ID NO: 245 is Anabaena_variabilis_ATCC29413, SEQ ID NO: 246 is Nodularia_spumigena, SEQ ID NO: 247 is Nostoc_punctiform, SEQ ID NO: 248 is Cyanothece_sp._PCC7425, SEQ ID NO: 249 is Thermosynechococcus_elongatus BP1, SEQ ID NO: 250 is Synechococcus_elongatus_PCC6301 and SEQ ID NO: 251 is Synechococcus_elongatus_PCC7942. FIG. 2B shows the CcmN, C-terminal alignment and secondary structure prediction of the targeting peptide region of the CcmN protein. SEQ ID NOs: 252-274 correspond to the targeting peptide region of the CcmN protein from Acaryochloris marina MBIC11017 (SEQ ID NO: 252), Trichodesmium erythraeum (SEQ ID NO: 253), Synechococcus elongatus PCC 6301 (SEQ ID NO: 254), Synechococcus elongatus PCC 794 (SEQ ID NO: 255), Gloeobacter violaceus (SEQ ID NO: 256), Synechococcus sp. JA-3-3Ab (SEQ ID NO: 257), Synechococcus sp. JA-2-3B′a(2-13) (SEQ ID NO: 258), Nodularia spumigena (SEQ ID NO: 259), Nostoc punctiforme (SEQ ID NO: 260), Anabaena variabilis (SEQ ID NO: 261), Nostoc sp PCC 7120 (SEQ ID NO: 262), Lyngbya sp PCC 8106 (SEQ ID NO: 263), Synechococcus sp PCC7002 (SEQ ID NO: 264), Microcystis aeruginosa (SEQ ID NO: 265), Cyanothece sp PCC8801 (SEQ ID NO: 266), Cyanothece sp PCC8802 (SEQ ID NO: 267), Cyanothece sp CCY0110 (SEQ ID NO: 268), Cyanothece sp ATCC51142 (SEQ ID NO: 269), Crocosphaera watsonii (SEQ ID NO: 270), Synechocystis sp PCC 6803 (SEQ ID NO: 271), Cyanothece sp PCC 7822 (SEQ ID NO: 272), Thermosynechococcus elongatus (SEQ ID NO: 273) and Cyanothece sp PCC 7425 (SEQ ID NO: 274).

FIG. 3A shows the alignment and secondary structure prediction of the N-terminal region of a pdu-associated aldehyde dehydrogenase (PduP) from 12 microorganisms including an ortholog of PduP (from Propionibacterium acnes) that is not associated with bacterial microcompartments and does not contain a targeting peptide. The N-terminal peptide of the Salmonella typhimurium LT2 PduP has been shown to target a pdu-type bacterial microcompartment in Fan et al. 2010. The helical wheel representation for this peptide is shown in FIG. 3C(II). The first sequence of the alignment is an ortholog of PduP that is not associated with bacterial microcompartments and therefore does not contain a targeting peptide. SEQ ID NOs: 194-205 correspond to Propionibacterium acnes J139 (SEQ ID NO: 194), Fusobacterium ulcerans ATCC 49185 (SEQ ID NO: 195), Escherichia coli CFT073 (SEQ ID NO: 196), Pectobacterium wasabiae WPP163 (SEQ ID NO: 197), Listeria monocytogenes 104035 (SEQ ID NO: 198), Shewanella sp W3-18-1 (SEQ ID NO: 199), Tolumonas aurensis DSM 9187(SEQ ID NO: 200), Yersinia frederiksenii ATCC 33641 (SEQ ID NO: 201), Klebsiella pneumoniae 342 (SEQ ID NO: 202), Salmonella typhimurium LT2 (SEQ ID NO: 203), Salmonella enterica Paratyphi B str. Sp87 (SEQ ID NO: 204) and Citrobacter koseri ATCC BAA 895 (SEQ ID NO: 205).

FIG. 3B shows an alignment overview of all BMC targeting peptides (305 unique sequences of N- and C-terminal and inter-domain peptides). All unique BMC targeting peptides are colored based on amino acid property with positional amino acid variations indicated as percentages and consensus amino acid properties at each position indicated. The position of the consensus predicted helix is indicated by the thick, black bar under residues 3-13.

Helical wheel representations of the targeting peptides in various organisms are shown in the figures. In the helical wheel representations of the predicted alpha helix on the left panel represents the predicted helical targeting peptide for the organism protein listed. Hydrophobic residues are represented as diamonds where the color scale is from dark gray, for most hydrophobic, with amount of gray decreasing proportionally to the hydrophobicity, to light gray. Hydrophilic residues are represented as circles where the color scale is from black, for most hydrophilic, with amount of black decreasing proportionally to the hydrophilicity, to light gray. Potential negatively charged residues are represented as triangles colored light gray. Potential positively charged residues are represented as pentagons colored light gray.

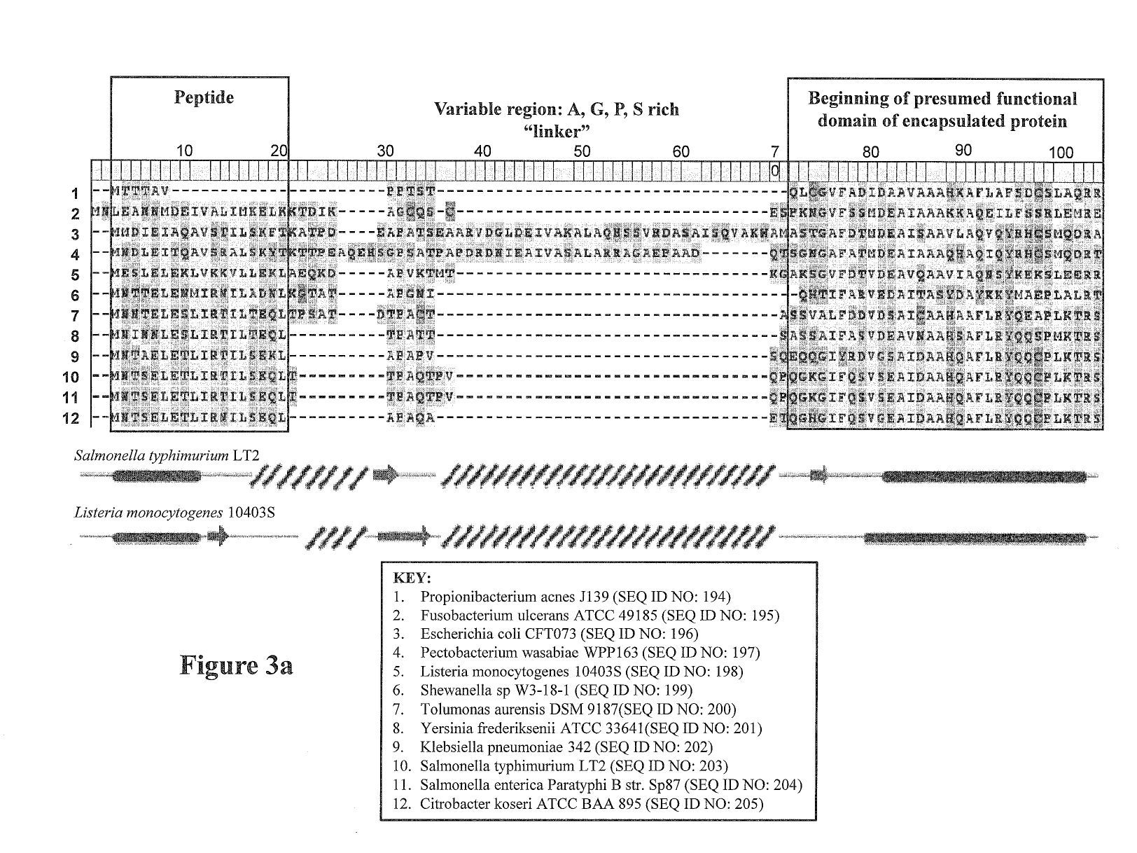

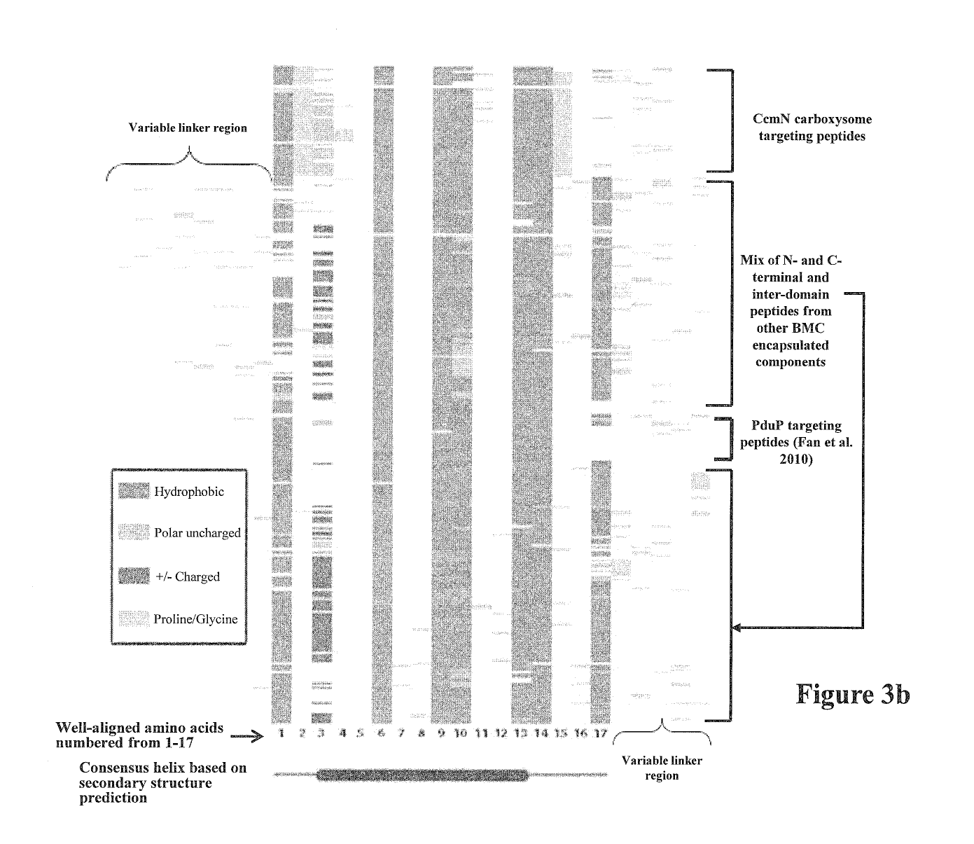

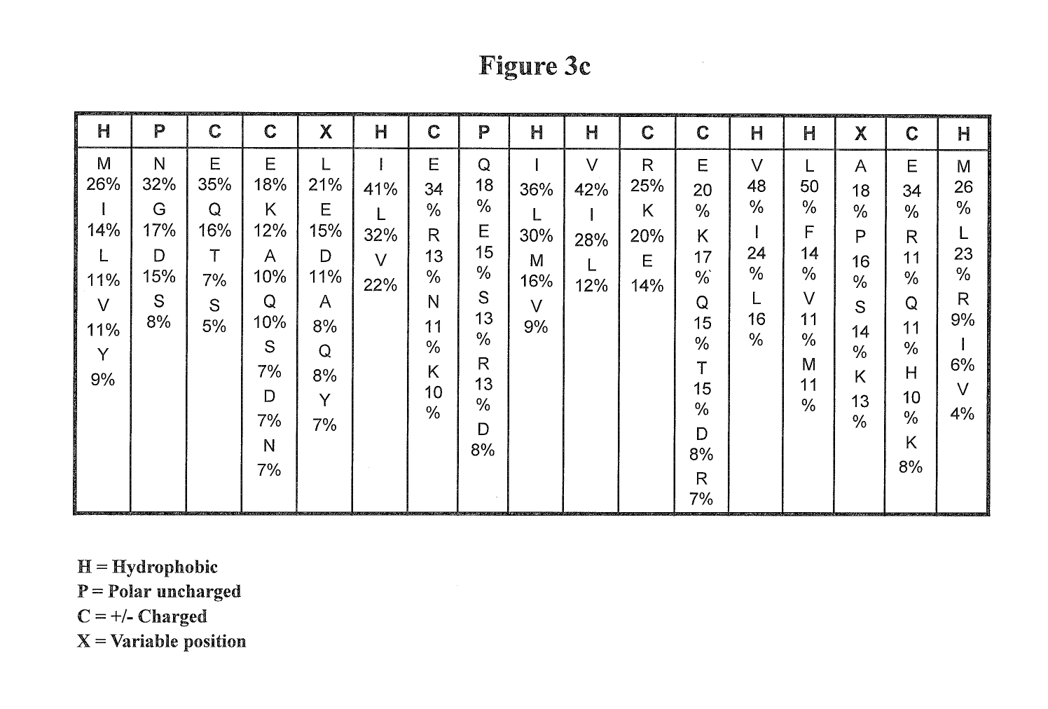

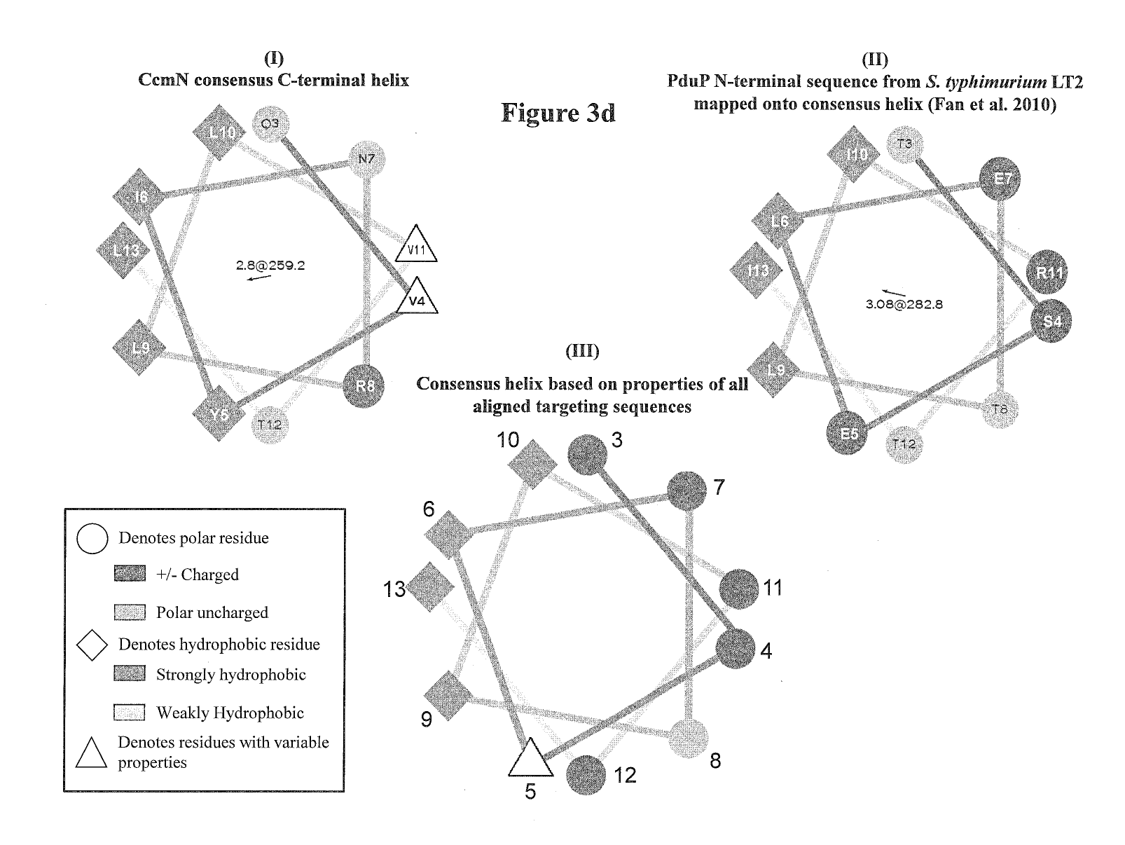

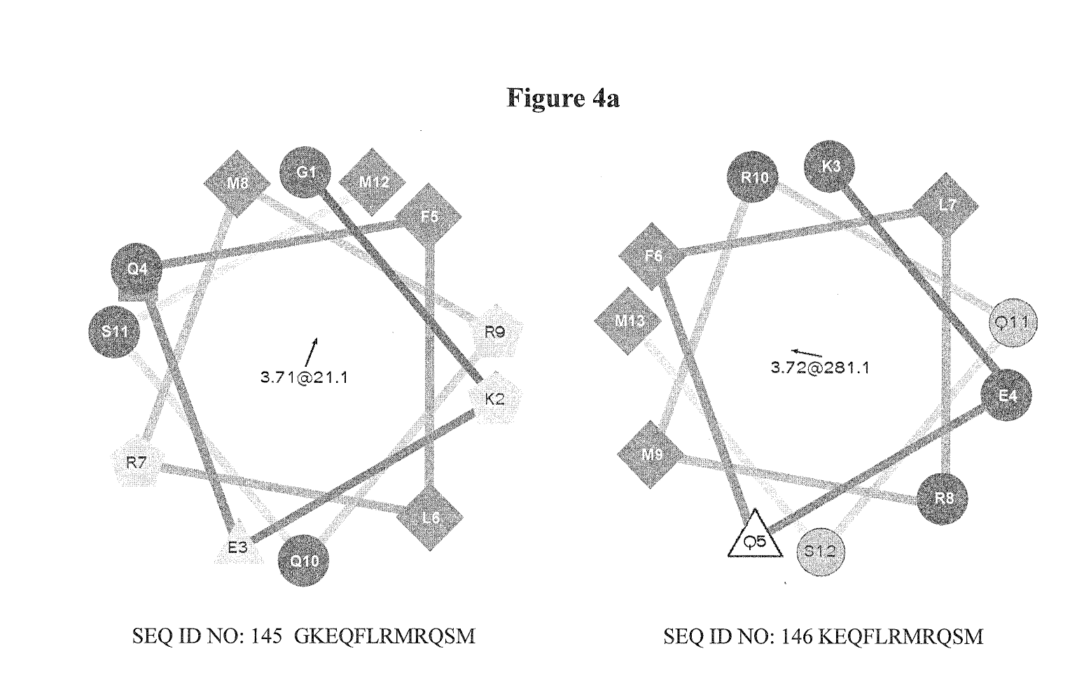

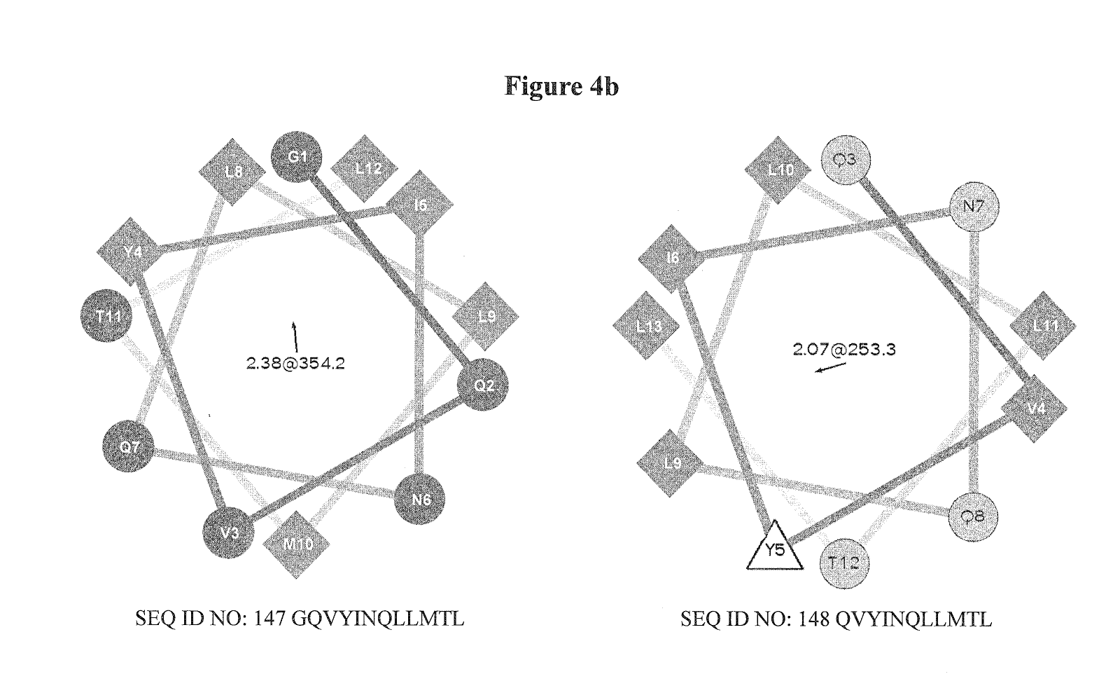

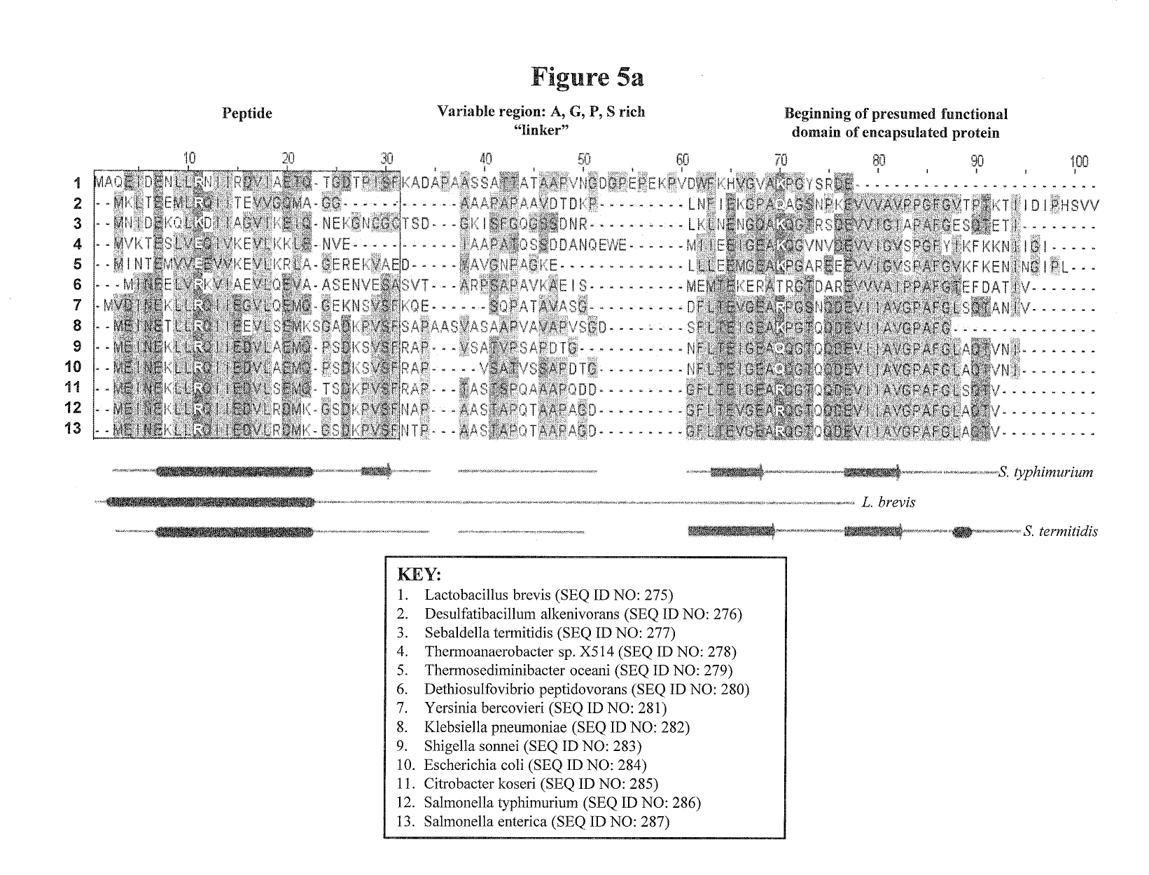

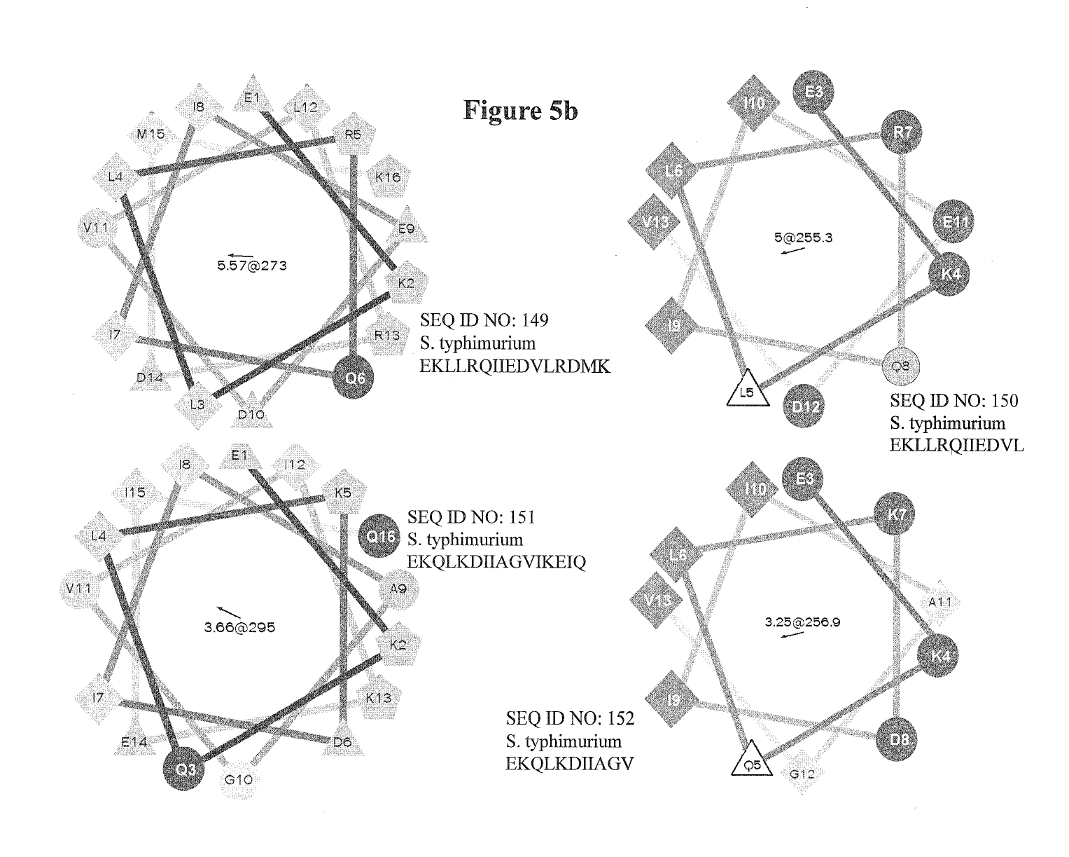

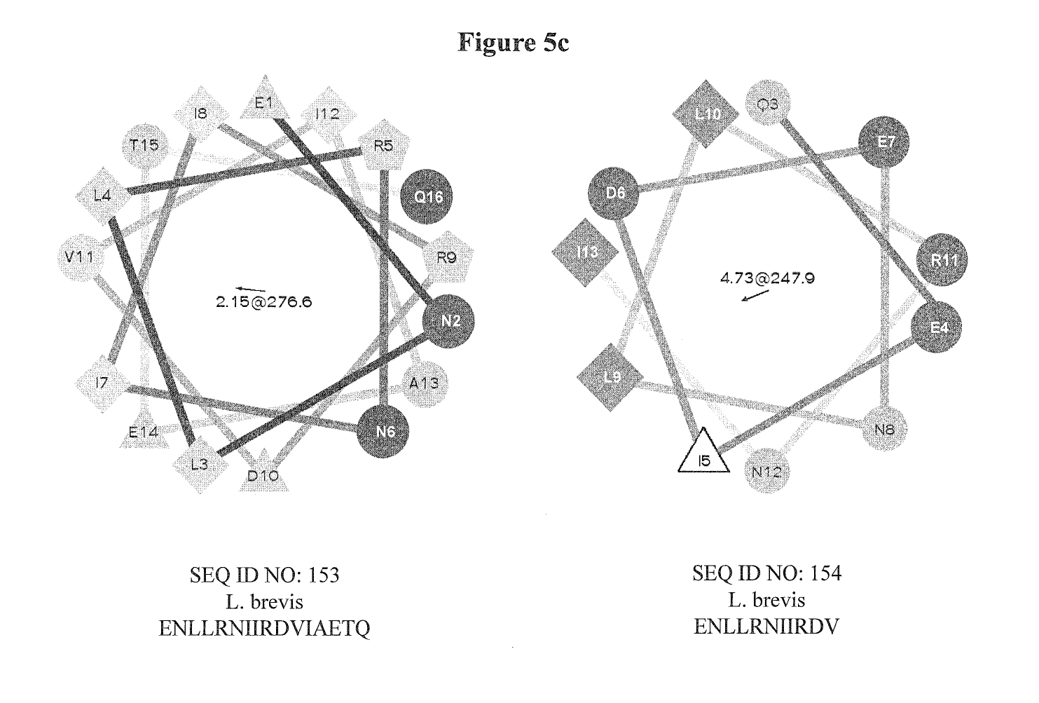

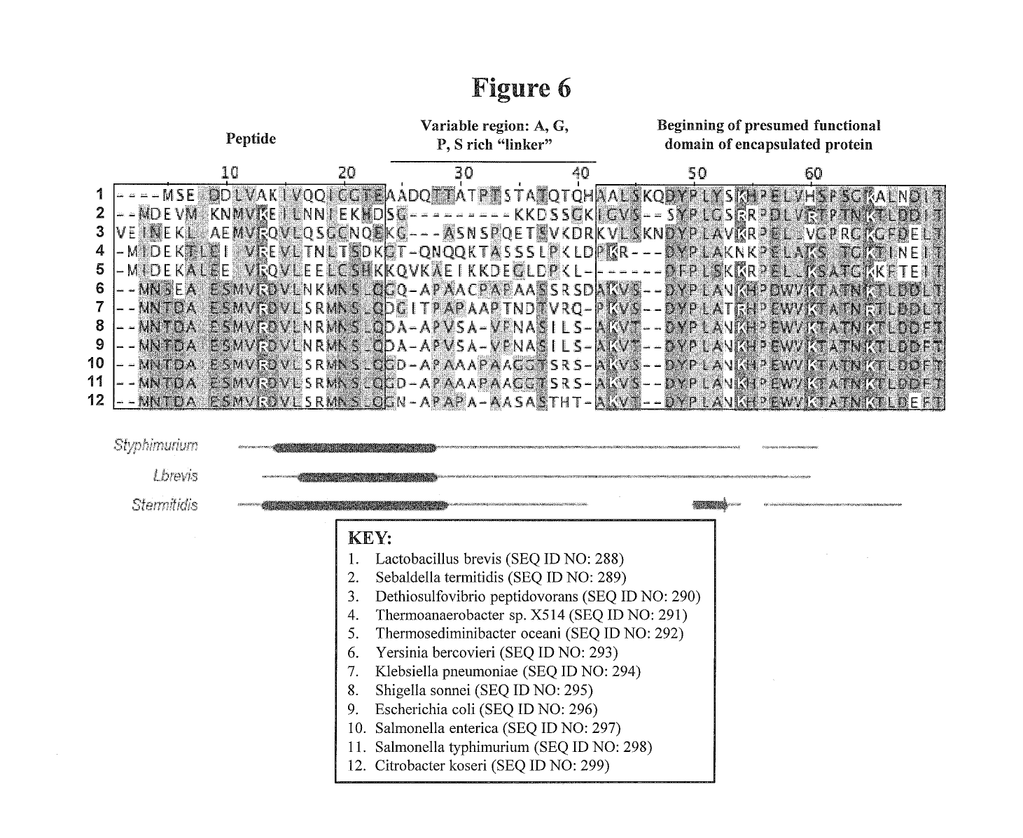

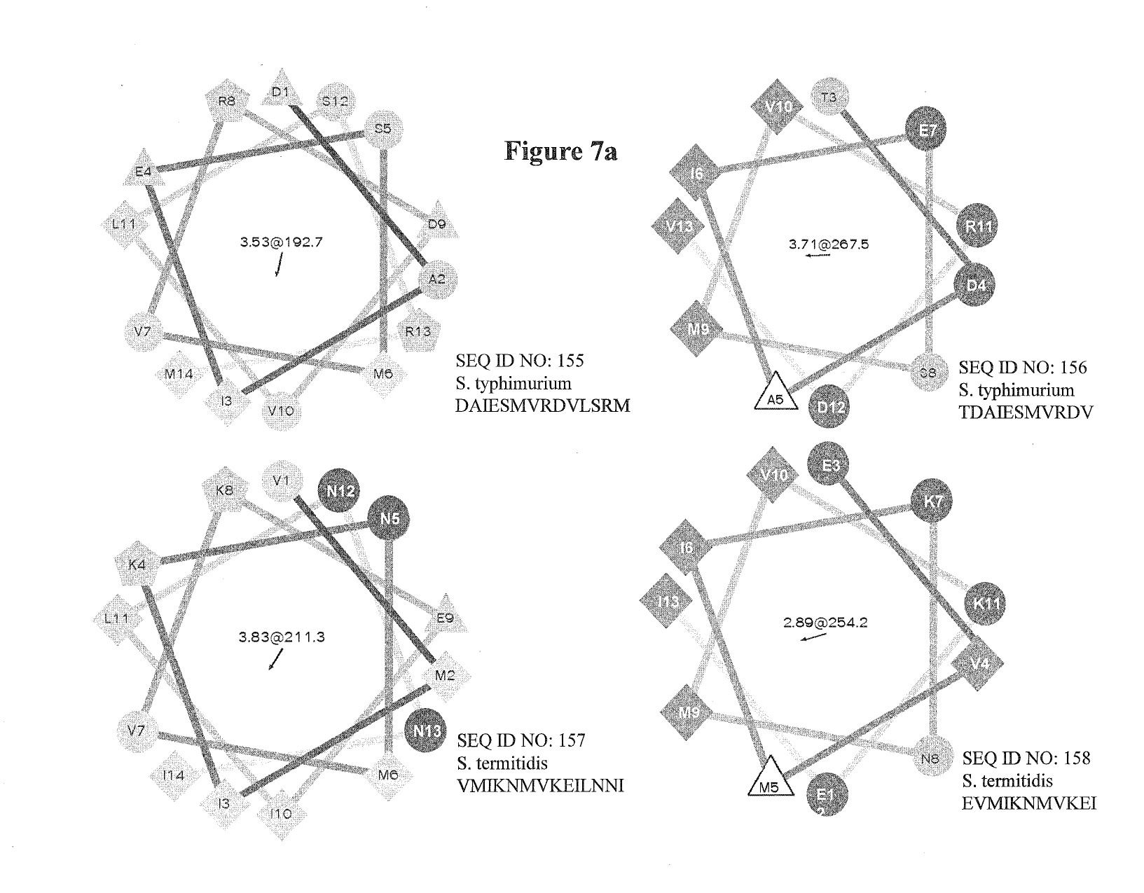

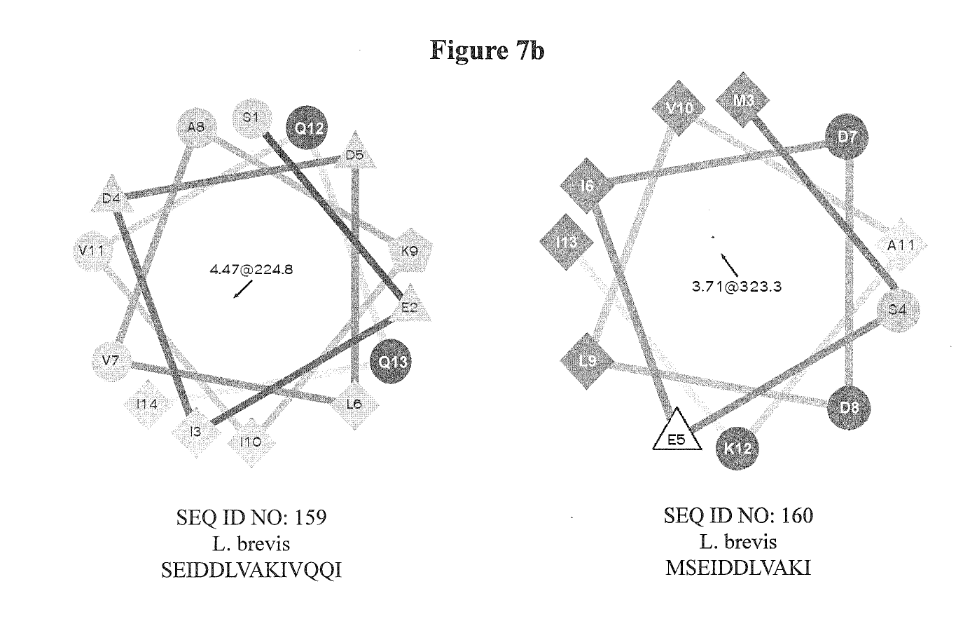

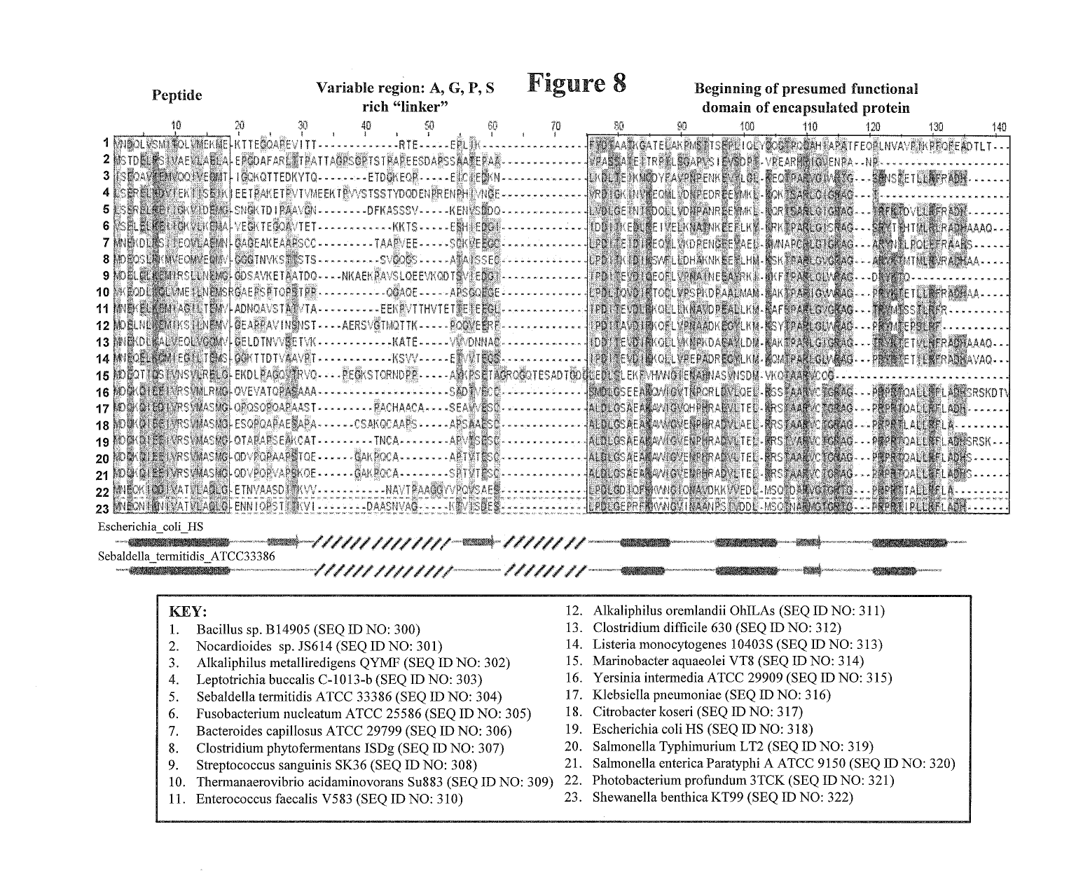

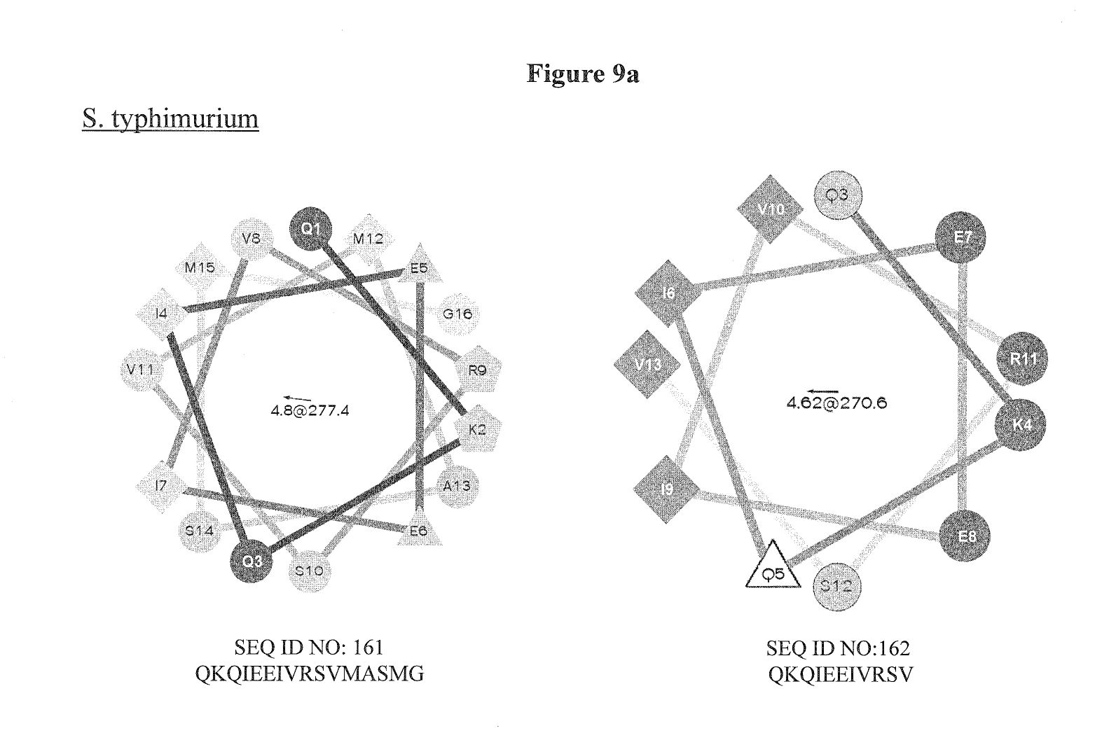

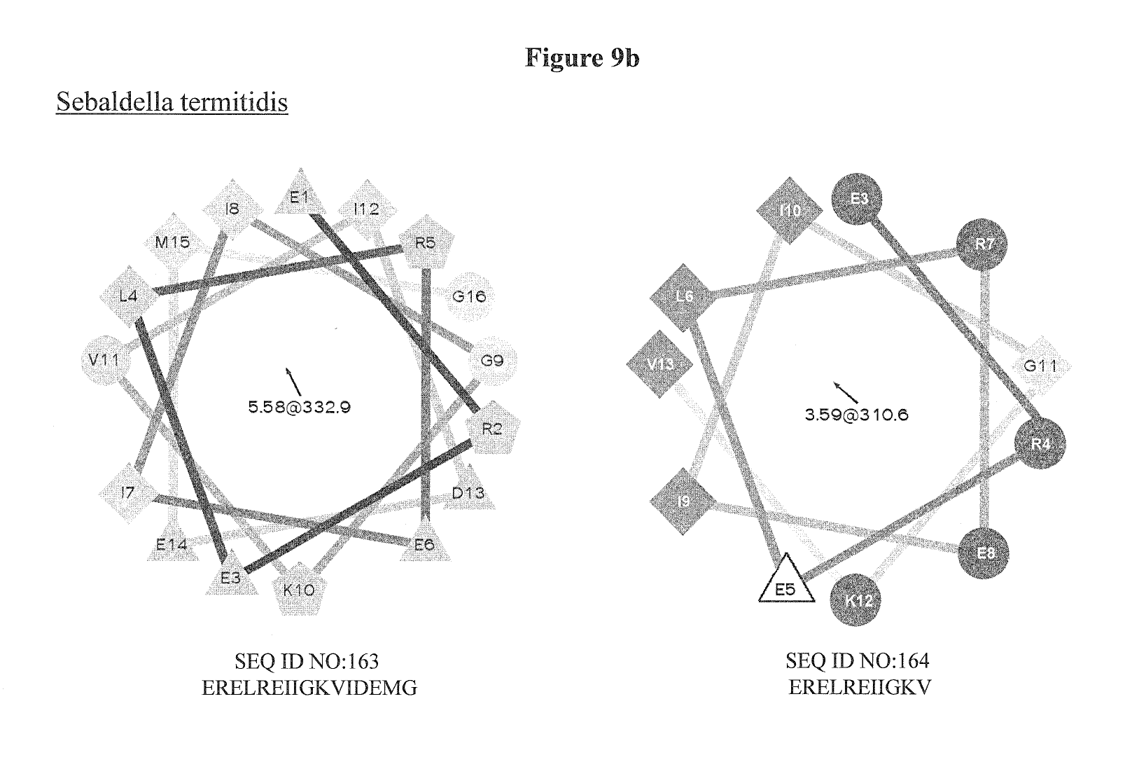

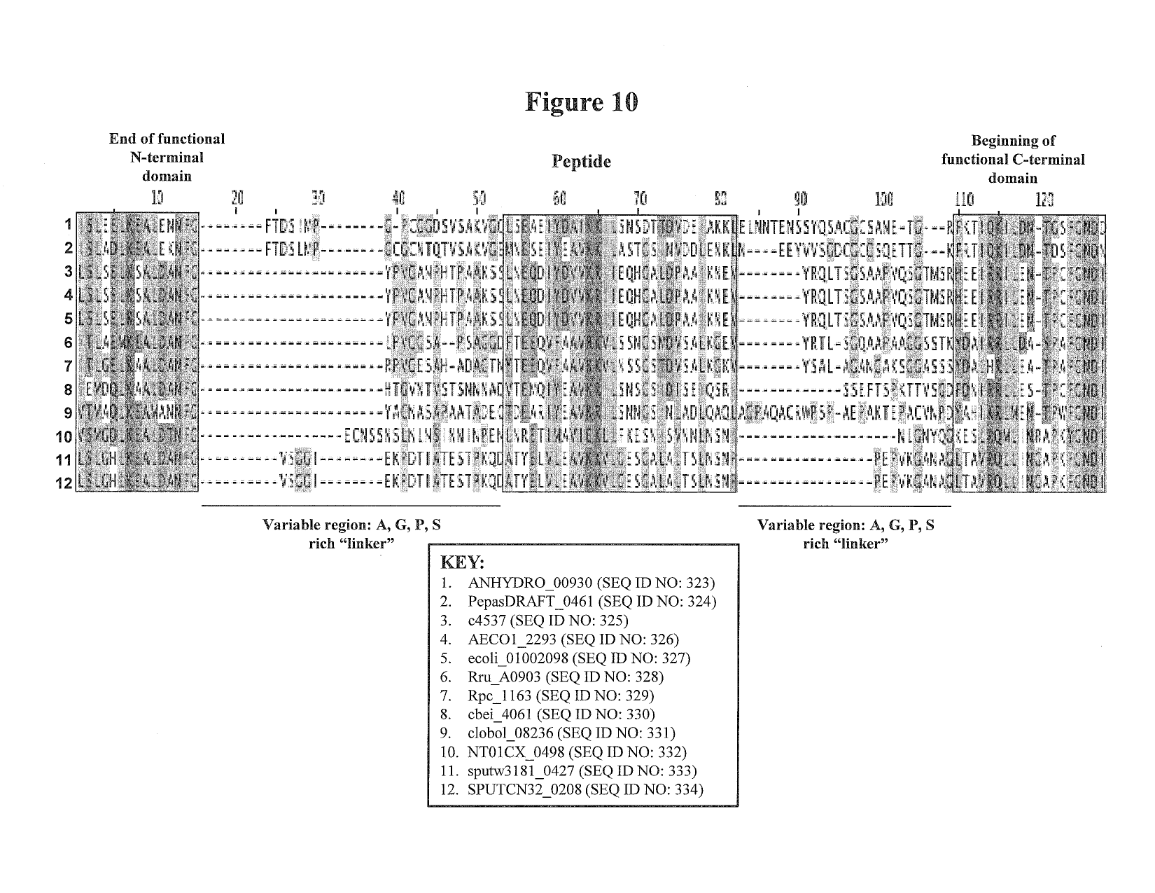

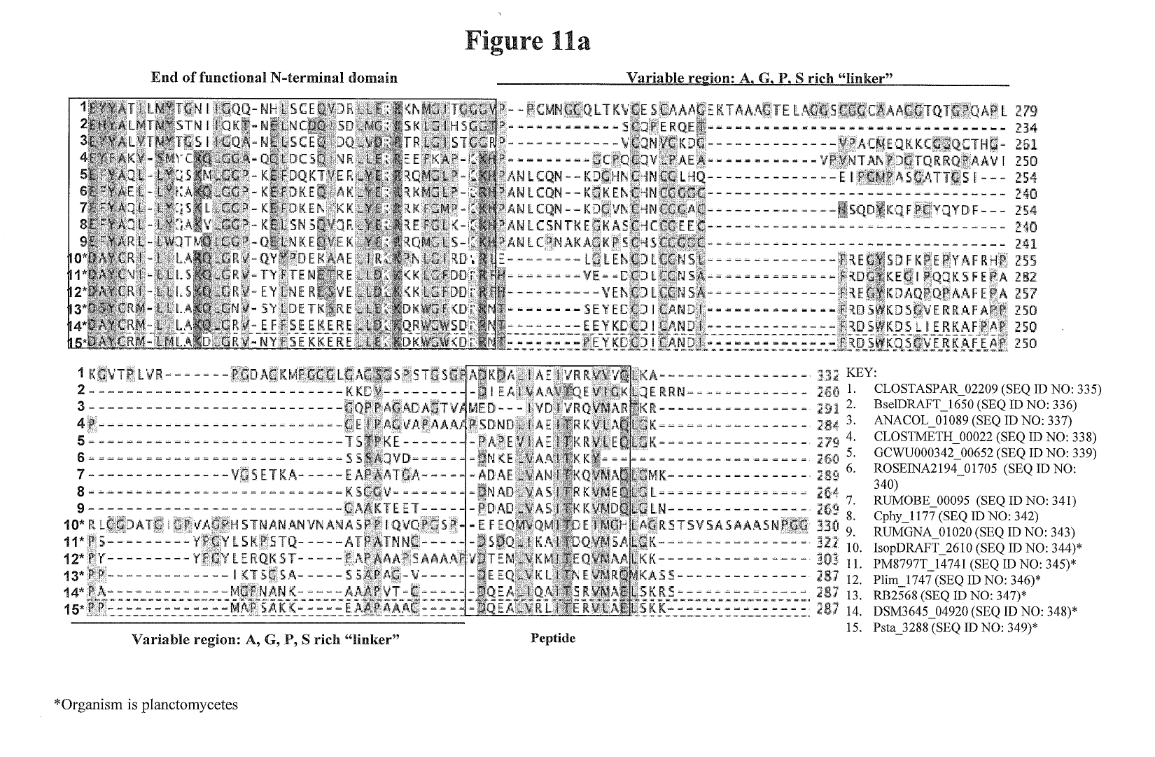

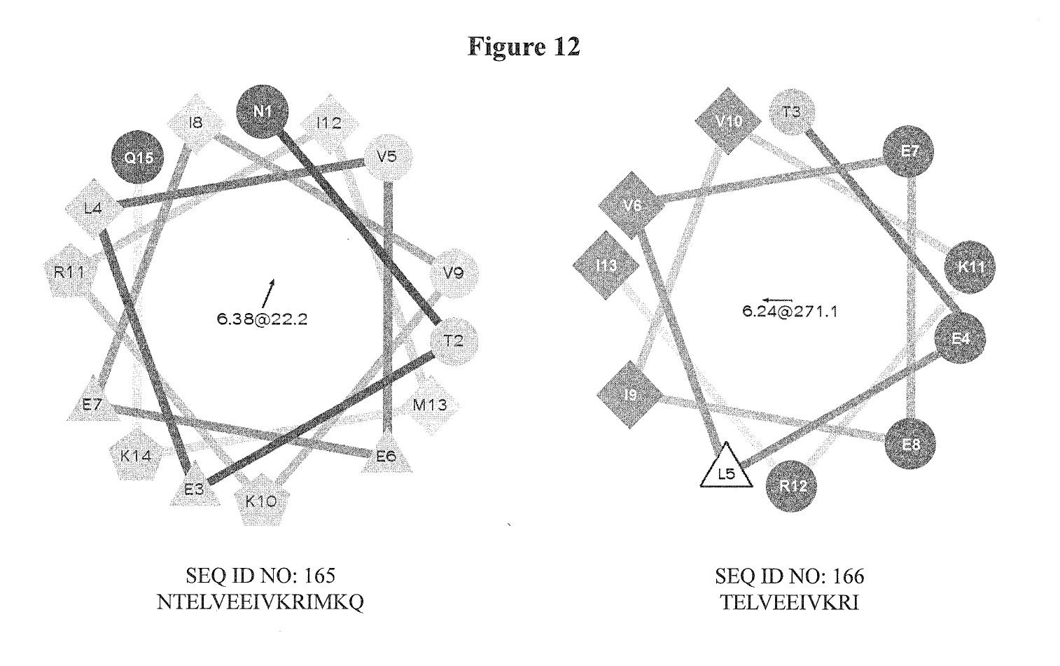

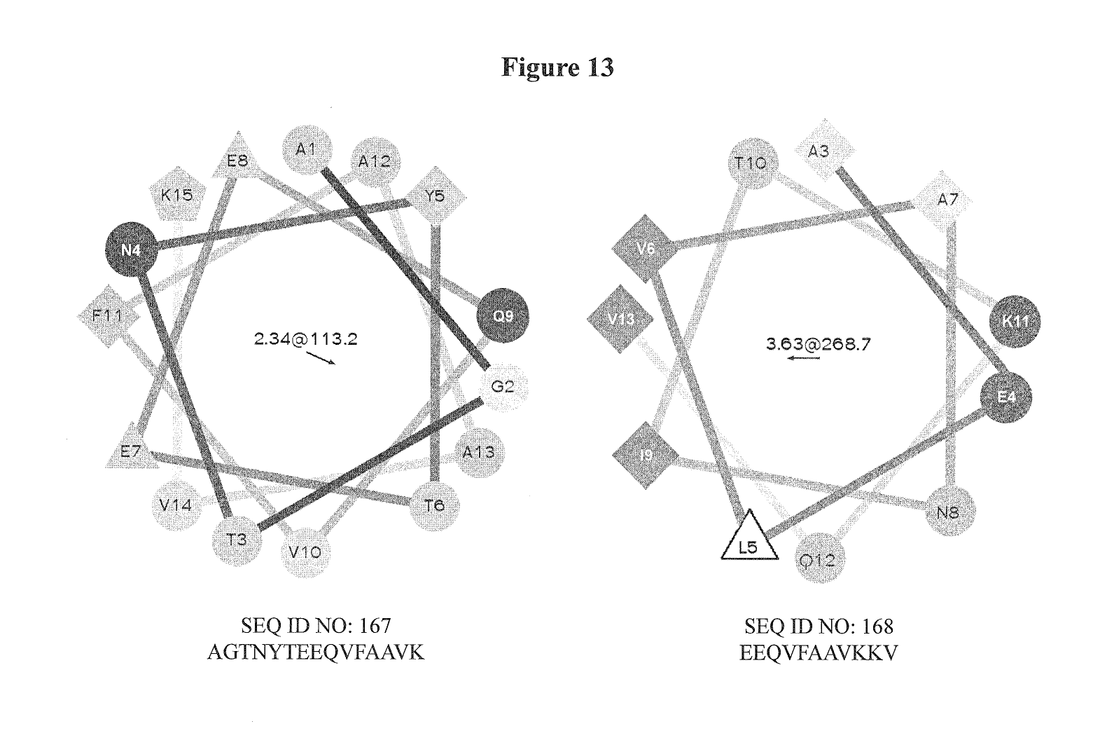

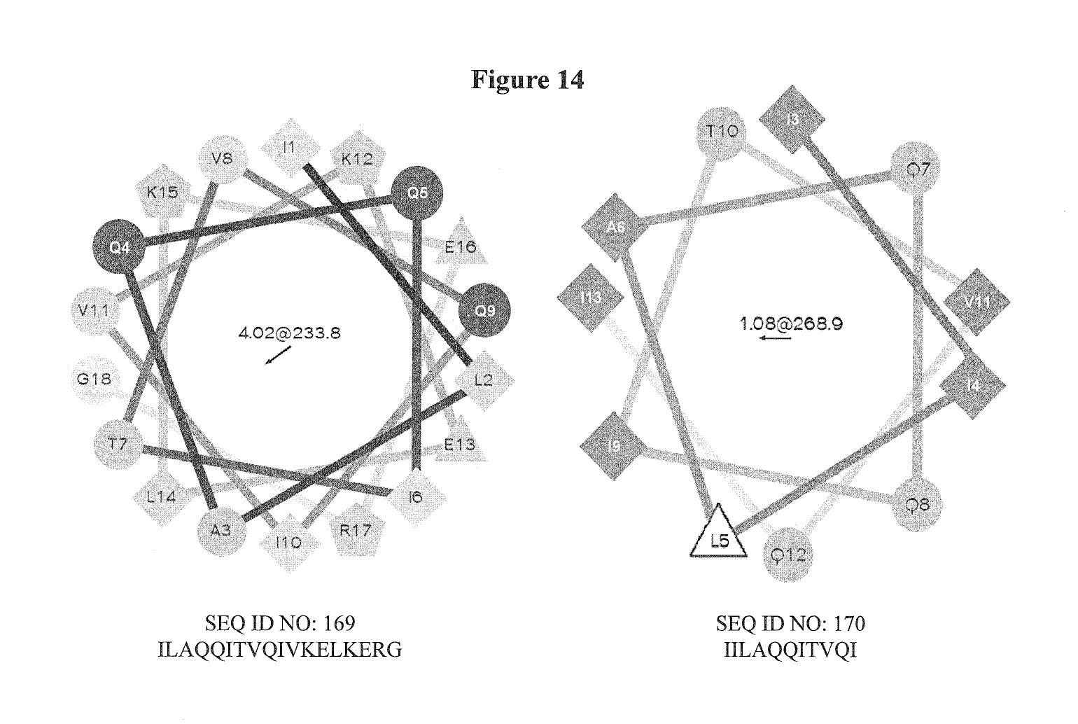

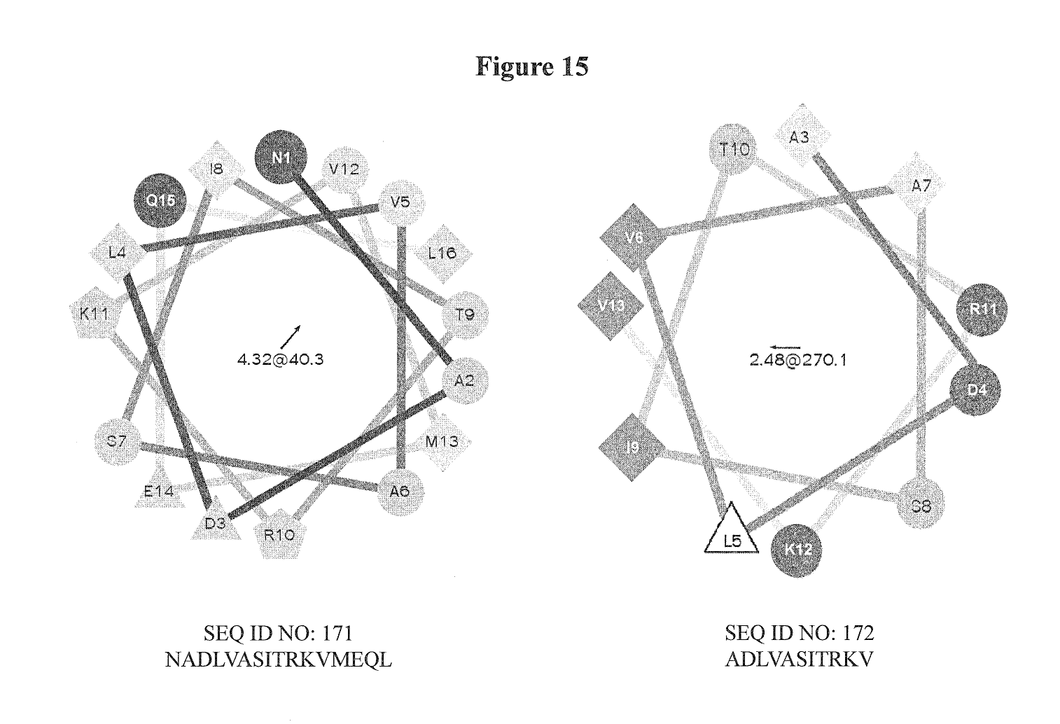

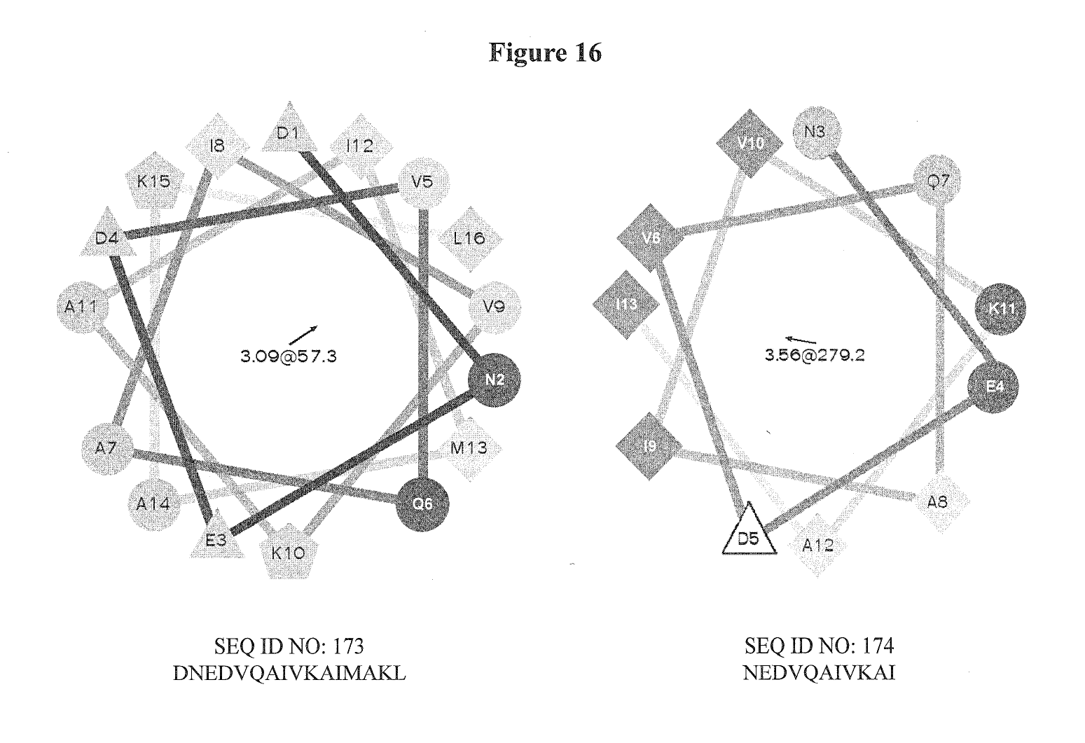

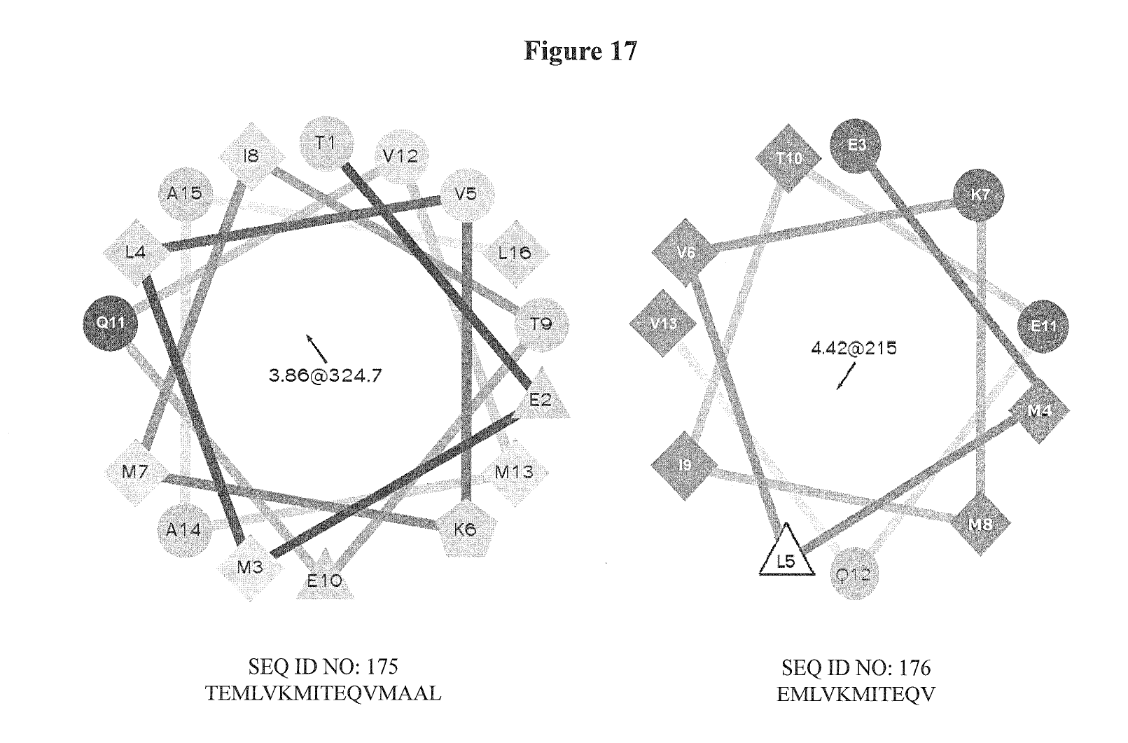

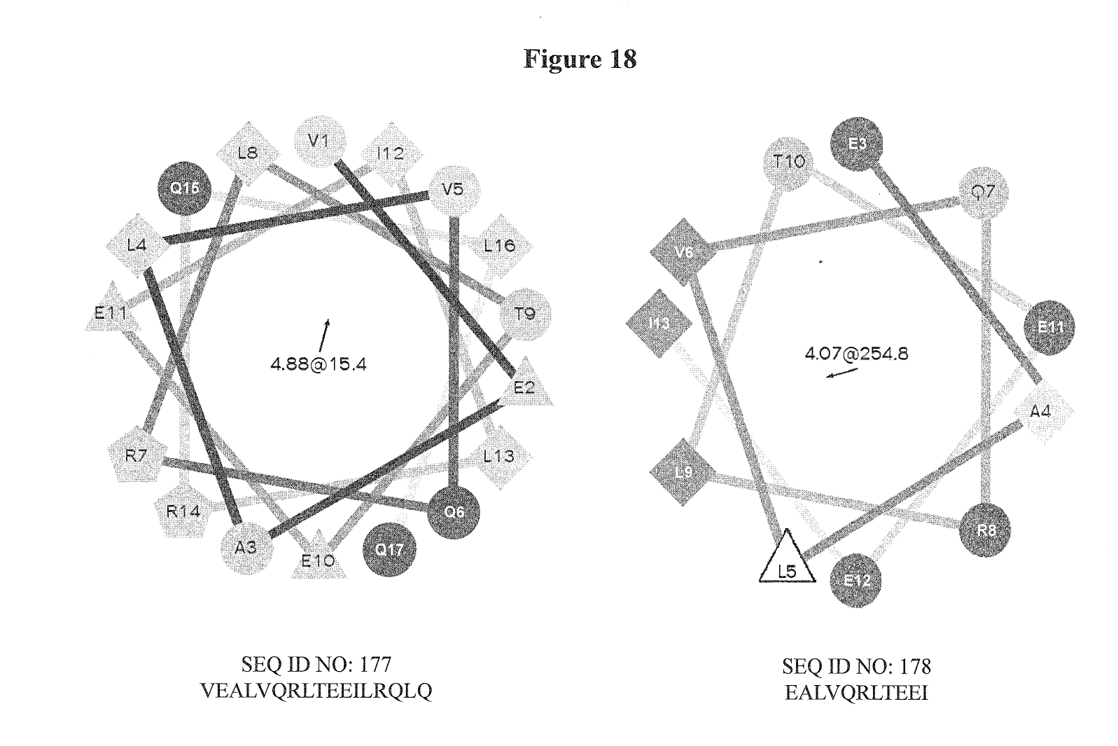

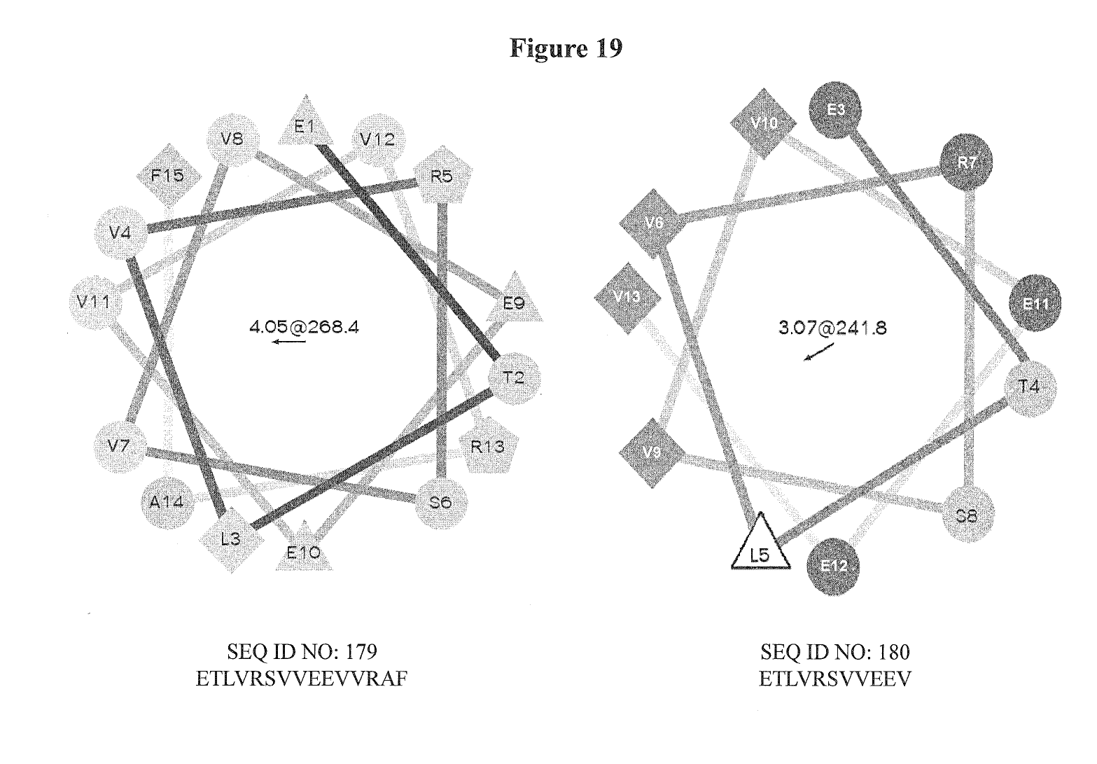

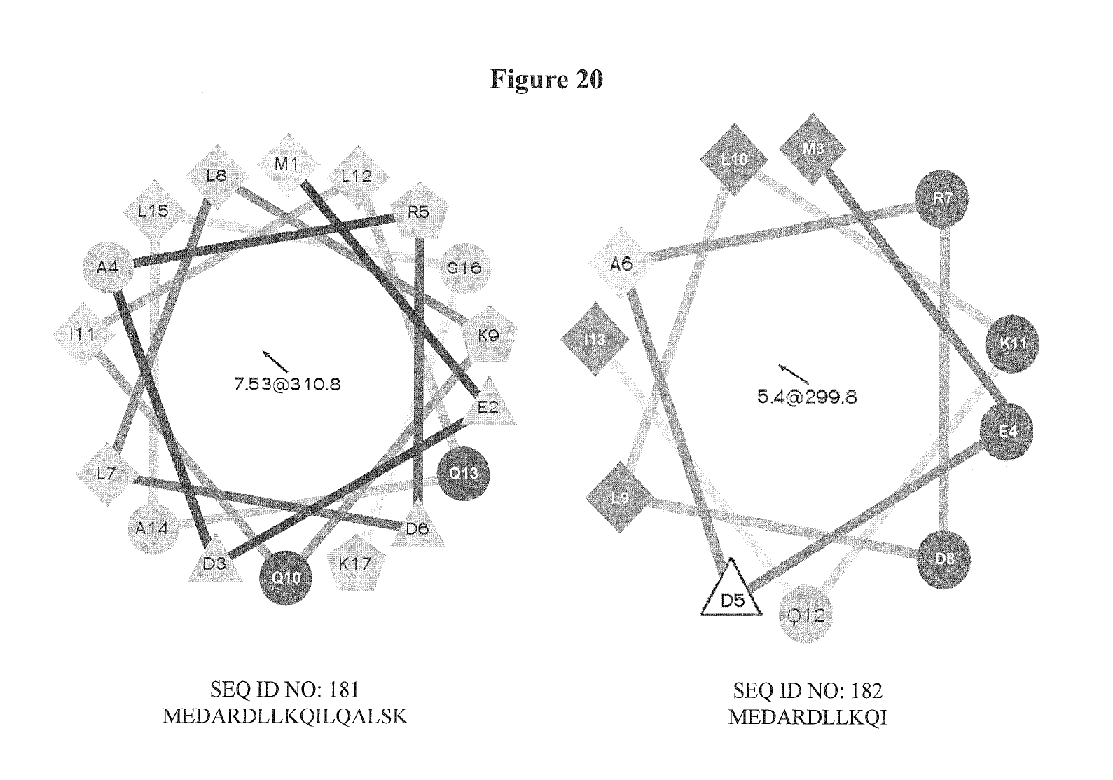

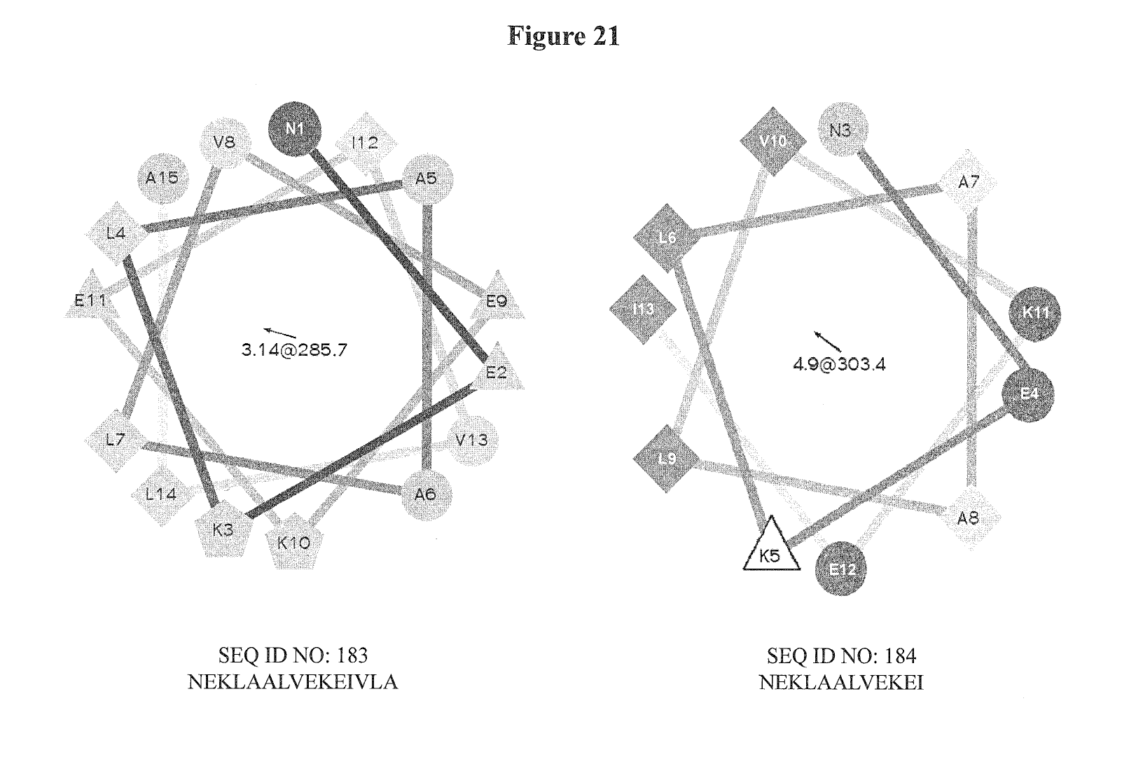

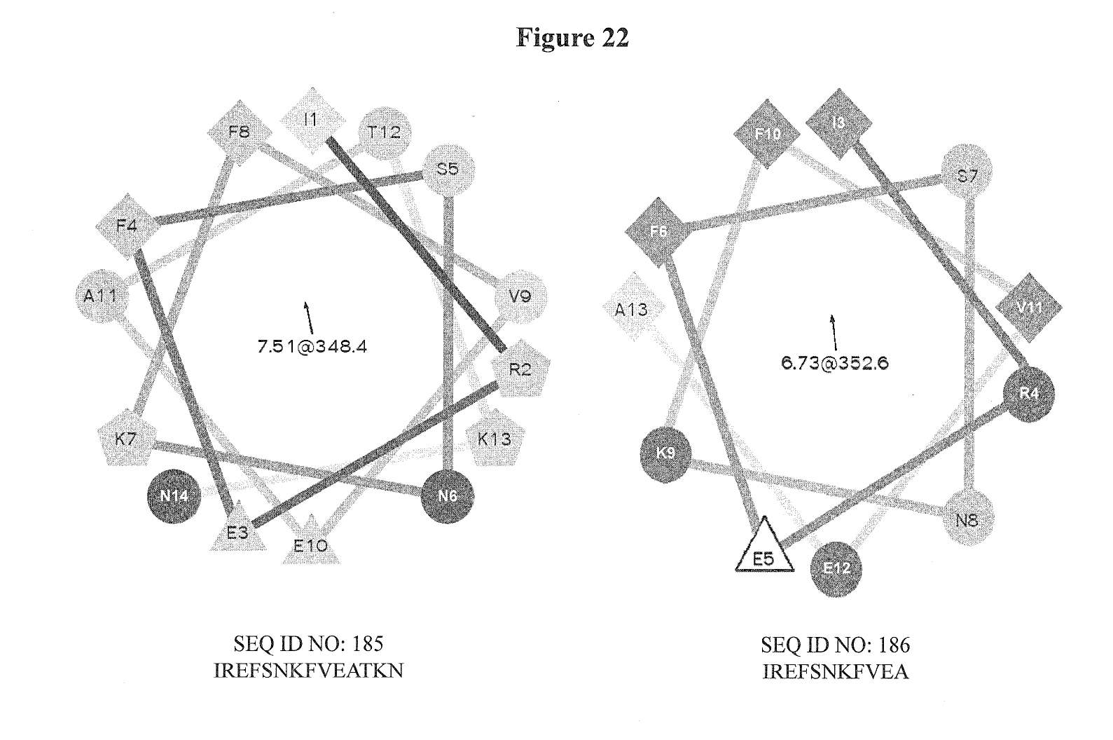

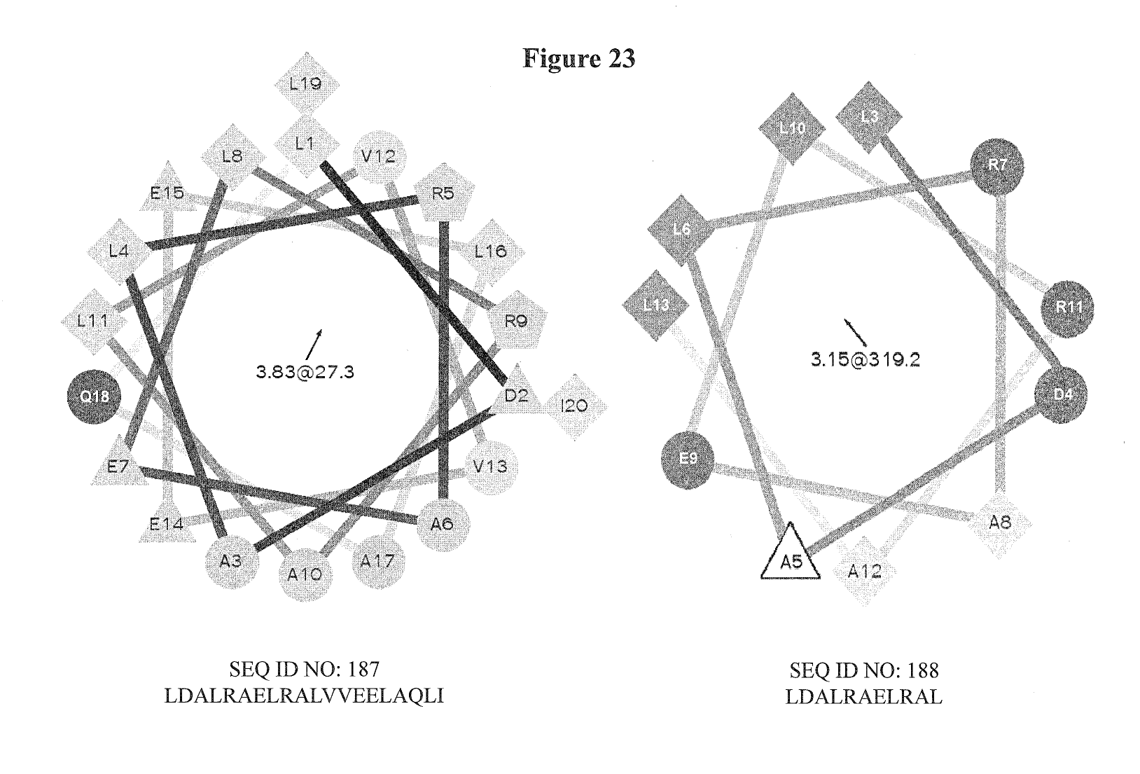

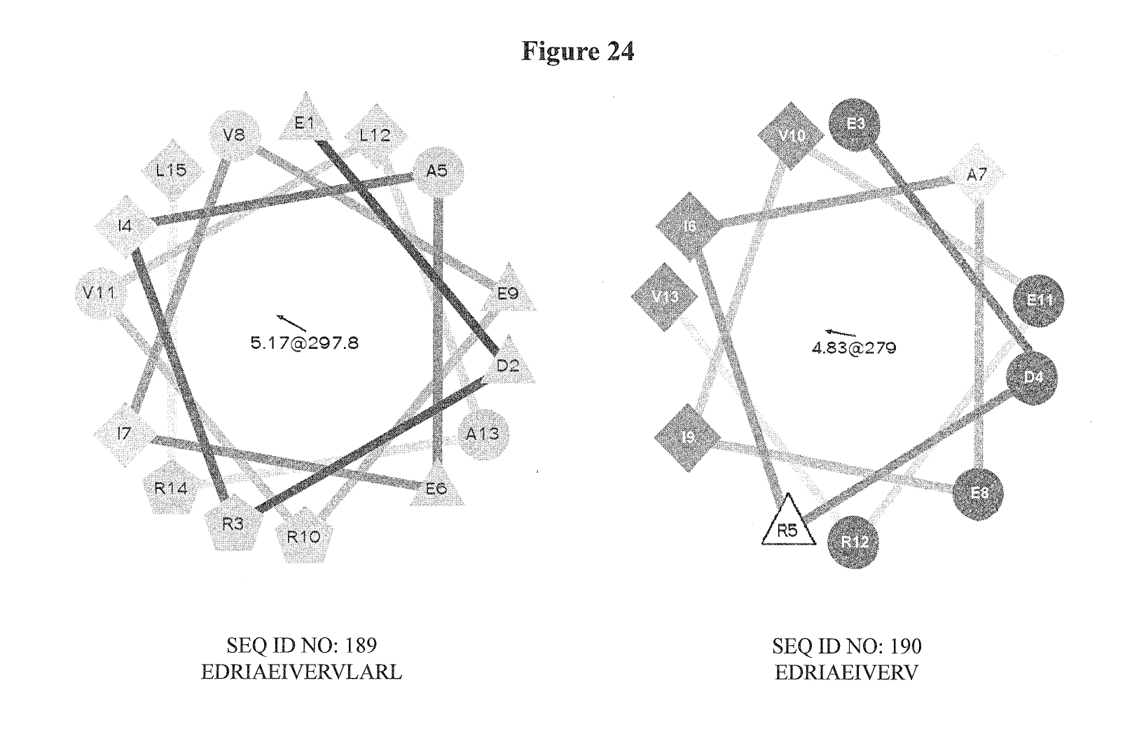

In the helical wheel representations shown in the figures, the alpha helix on the right panel of each figure represents the portion of the predicted helical targeting peptide for the organism as mapped onto the consensus helical wheel prediction for all targeting peptides shown in FIG. 3C and using the scheme shown in FIG. 3D. Hydrophobic residues are represented as diamonds. Hydrophilic residues are represented as circles where light gray shading represents polar uncharged residues and dark gray shading represents positively or negatively charged residues. In the consensus helical wheel representations, positions with variable amino acid composition are denoted with a triangle.

FIG. 3C shows the consensus peptide motif Majority amino acid percentages at each well-aligned position were calculated in Jalview. Amino acid property at each position was given based on the majority amino acid property (H=hydrophobic, C=charged, P=polar) at each aligned position. Positions 5 and 15 were highly variable based on identity and property and no consensus property denoted by an X.

FIG. 3D describes mapping of consensus residues and known PduP targeting sequence onto consensus helix prediction. Panel I shows a portion of the consensus sequence of the CcmN C-terminal peptide mapped onto a helical wheel diagram based on a consensus helix prediction for all BMC targeting peptides. Panel II shows a portion of the known targeting peptide sequence from PduP (Fan et al. 2010) mapped onto the consensus helix and the consensus amino acid property at each position based on the alignment of all BMC targeting peptides (FIG. 3B) mapped on the consensus helix. The numbering is based on the 17 well-aligned residues shown in the motif in FIG. 3C. Panel III shows the consensus helix based on properties of all aligned targeting sequences.

FIGS. 4A and B show helical wheel projection of the predicted alpha helix of the C-terminal region of CcmN of Synechococcus elongatus PCC7942 (SEQ ID NO: 145 and 146) and Synechocystis PCC 6803 (SEQ ID NO: 147-148).

FIG. 5A shows the alignment and secondary structure prediction of the N-terminal region of Diol dehydratase medium subunit (PduD) from 13 microorganisms. SEQ ID NOs: 275-287 correspond to Lactobacillus brevis (SEQ ID NO: 275), Desulfatibacillum alkenivorans (SEQ ID NO: 276), Sebaldella termitidis (SEQ ID NO: 277), Thermoanaerobacter sp. X514 (SEQ ID NO: 278), Thermosediminibacter oceani (SEQ ID NO: 279), Dethiosulfovibrio peptidovorans (SEQ ID NO: 280), Yersinia bercovieri (SEQ ID NO: 281), Klebsiella pneumoniae (SEQ ID NO: 282), Shigella sonnei (SEQ ID NO: 283), Escherichia coli (SEQ ID NO: 284), Citrobacter koseri (SEQ ID NO: 285), Salmonella typhimurium (SEQ ID NO: 286) and Salmonella enterica (SEQ ID NO: 287). FIGS. 5B and 5C shows helical wheel projections of a peptide from the diol dehydratase medium subunit (PduD) N-terminal region in Salmonella typhimurium (SEQ ID NO: 149-152) and Lactobacillus brevis (SEQ ID NO: 153-154). The peptides shown fall within the protein sequences are shown boxed in FIG. 5A. The peptides on the right panels in FIGS. 5B and 5C are helical wheel projections of the portion of the predicted peptide that map onto the consensus helical wheel prediction for all peptides.

FIG. 6 shows the alignment and secondary structure prediction of the N-terminal region of diol dehydratase small subunit (PduE) from 11 microorganisms. SEQ ID NOs: 288-299 correspond to: Lactobacillus brevis (SEQ ID NO: 288), Sebaldella termitidis (SEQ ID NO: 289), Dethiosulfovibrio peptidovorans (SEQ ID NO: 290), Thermoanaerobacter sp. X514 (SEQ ID NO: 291), Thermosediminibacter oceani (SEQ ID NO: 292), Yersinia bercovieri (SEQ ID NO: 293), Klebsiella pneumoniae (SEQ ID NO: 294), Shigella sonnei (SEQ ID NO: 295), Escherichia coli (SEQ ID NO: 296), Salmonella enterica (SEQ ID NO: 297), Salmonella typhimurium (SEQ ID NO: 298) and Citrobacter koseri (SEQ ID NO: 299).

FIGS. 7A and 7B shows the helical wheel projections of the N-terminal region (boxed in FIG. 6) from the diol dehydratase small subunit (PduE) in S. typhimurium (SEQ ID NO: 155-156), S. termitidis (SEQ ID NO: 157-158) and L. brevis (SEQ ID NO: 159 and 160) on the left hand side of the figures. The region of the peptides within the protein sequences are shown boxed in FIG. 6. The peptides on the right panels in FIGS. 7A and 7B are helical wheel projections of the portion of the predicted peptide that map onto the consensus helical wheel prediction for all peptides.

FIG. 8 shows the alignment and secondary structure prediction of the N-terminal region of the EutC (Ammonia lyase light chain) N-terminal region from 23 microorganisms. SEQ ID NOs : 300-322 corresponds to: Bacillus sp. B14905 (SEQ ID NO: 300), Nocardioides sp. JS614 (SEQ ID NO: 301), Alkaliphilus metalliredigens QYMF (SEQ ID NO: 302), Leptotrichia buccalis C-1013-b (SEQ ID NO: 303), Sebaldella termitidis ATCC 33386 (SEQ ID NO: 304), Fusobacterium nucleatum ATCC 25586 (SEQ ID NO: 305), Bacteroides capillosus ATCC 29799 (SEQ ID NO: 306), Clostridium phytofermentans ISDg (SEQ ID NO: 307), Streptococcus sanguinis SK36 (SEQ ID NO: 308), Thermanaerovibrio acidaminovorans Su883 (SEQ ID NO: 309), Enterococcus faecalis V583 (SEQ ID NO: 310), Alkaliphilus oremlandii OhILAs (SEQ ID NO: 311), Clostridium difficile 630 (SEQ ID NO: 312), Listeria monocytogenes 10403S (SEQ ID NO: 313), Marinobacter aquaeolei VT8 (SEQ ID NO: 314), Yersinia intermedia ATCC 29909 (SEQ ID NO: 315), Klebsiella pneumoniae (SEQ ID NO: 316), Citrobacter koseri (SEQ ID NO: 317), Escherichia coli HS (SEQ ID NO: 318), Salmonella Typhimurium LT2 (SEQ ID NO: 319), Salmonella enterica Paratyphi A ATCC 9150 (SEQ ID NO: 320), Photobacterium profundum 3TCK (SEQ ID NO: 321) and Shewanella benthica KT99 (SEQ ID NO: 322).

FIGS. 9A and 9B shows the helical wheel projections of targeting peptides from the EutC N-terminal helix region in S. typhimurium (SEQ ID NO: 161-162), and S. termitidis (SEQ ID NO: 163-164). The region of the peptides in the native sequence is shown boxed in FIG. 8 and the predicted helical targeting peptides are shown on the left panels. The peptides on the right panels in FIGS. 9A and 9B are helical wheel projections of the portion of the predicted peptide that map onto the consensus helical wheel prediction for all peptides

FIG. 10 shows the alignment and secondary structure prediction of B12-independent diol dehydratase showing interdomain peptide (Group 4). SEQ ID NOs: 323-334 correspond to ANHYDRO_00930 (SEQ ID NO: 323), PepasDRAFT_0461 (SEQ ID NO: 324), c4537 (SEQ ID NO: 325), AECO1_2293 (SEQ ID NO: 326), ecoli_01002098 (SEQ ID NO: 327), Rru_A0903 (SEQ ID NO: 328), Rpc_1163 (SEQ ID NO: 329), cbei_4061 (SEQ ID NO: 330), clobol_08236 (SEQ ID NO: 331), NT01CX_0498 (SEQ ID NO: 332), sputw3181_0427 (SEQ ID NO: 333) and SPUTCN32_0208 (SEQ ID NO: 334).

FIG. 11A shows the alignment and secondary structure prediction of L-Fuculose phosphate aldolase C-terminal region (peptide) presumed to be encapsulated in BMCs of some Planctomycetes and selection of Firmicutes. SEQ ID NOs: 335-359 corresponds to CLOSTASPAR_02209 (SEQ ID NO: 335), BselDRAFT_1650 (SEQ ID NO: 336), ANACOL_01089 (SEQ ID NO: 337), CLOSTMETH_00022 (SEQ ID NO: 338), GCWU000342_00652 (SEQ ID NO: 339), ROSEINA2194_01705 (SEQ ID NO: 340), RUMOBE_00095 (SEQ ID NO: 341), Cphy_1177 (SEQ ID NO: 342), RUMGNA_01020 (SEQ ID NO: 343), IsopDRAFT_2610 (SEQ ID NO: 344), PM8797T_14741 (SEQ ID NO: 345), Plim_1747 (SEQ ID NO: 346), RB2568 (SEQ ID NO: 347), DSM3645_04920 (SEQ ID NO: 348) and Psta_3288 (SEQ ID NO: 349).

FIGS. 12-24 shows the helical wheel projection for various peptides from various organisms The helical wheel representative peptide on the right panel in FIGS. 12-24 are the fragments of the larger peptide shown in the left, mapped onto the consensus peptide motif shown in FIGS. 3B and C according to the scheme described in FIG. 3D.

FIG. 12 shows the EutE homologue from C. phytofermentans C-terminal peptide helical wheel representative peptides (SEQ ID NO: 165 and 166).

FIG. 13 shows the B12-independent propanediol dehydratase from R. palustris BisB18 Interdomain-linker peptide helical wheel representations (SEQ ID NO: 167 and 168).

FIG. 14 shows the B12-independent propanediol dehydratase from C. phytofermentans Interdomain-linker peptide helical wheel representations (SEQ ID NO: 169 and 170).

FIG. 15 shows the Fuculose phosphate aldolase from C. phytofermentans C-terminal peptide helical wheel representations (SEQ ID NO: 171 and 172).

FIG. 16 shows the Aldehyde dehydrogenase from C. kluyveri C-terminal peptide helical wheel representations (SEQ ID NO: 173 and 174).

FIG. 17 shows the Fuculose phosphate aldolase from P. limnophilus C-terminal peptide helical wheel representations (SEQ ID NO: 175 and 176).

FIG. 18 shows the Fuculose/rhamnose phosphate aldolase from O. terrae PB90-1 C-terminal peptide helical wheel representations (SEQ ID NO: 177 and 178).

FIG. 19 shows the Aldehyde dehydrogenase from O. terrae PB90-1 N-terminal peptide helical wheel representations (SEQ ID NO: 179 and 180).

FIG. 20 shows the Aldehyde dehydrogenase (Cphy_1416) from C. phytofermentans C-terminal peptide helical wheel representations (SEQ ID NO: 181 and 182).

FIG. 21 shows the Aldehyde dehydrogenase (Cphy_1428) from C. phytofermentans C-terminal peptide helical wheel representations (SEQ ID NO: 183 and 184).

FIG. 22 shows the Unknown glycyl radical enyzme (Cphy_1417) from C. phytofermentans N-terminal peptide helical wheel representations (SEQ ID NO: 185 and 186).

FIG. 23 shows the Aldehyde dehydrogenase from M. smegmatis C-terminal peptide helical wheel representations (SEQ ID NO: 187 and 188).

FIG. 24 shows the Aldehyde dehydrogenase from H. ochraceum N-terminal peptide helical wheel representations (SEQ ID NO: 189 and 190).

Table 4 is a compilation of Tables 1-3 plus additional notes and information.

### DETAILED DESCRIPTION

### Introduction

Bacterial microcompartments (BMCs) encapsulate functionally related proteins. The bacterial microcompartment shell is composed of multiple paralogs of proteins containing the BMC domain (Pfam 00936) and presumably a relatively small number of proteins containing the Pfam03319 domain. There is recognizable sequence homology among the >2000 BMC domains in the sequence databases, suggesting that despite functional diversity and some differences in the morphology of a specific bacterial microcompartment type, there are conserved structural determinants for targeting and binding of the enzymes and auxiliary proteins that are encapsulated in BMCs.

BMC shell proteins and the components they encapsulate are typically found in gene clusters (putative operons). We have identified a common region of primary structure on a subset of the proteins presumed to be encapsulated in functionally diverse BMCs. The common region is ˜20 amino acids long and is located at either the N- or the C-terminus of encapsulated proteins, and in a few cases, in between domains of a single protein. This peptide is separated from the rest of the protein by a poorly conserved linker region that is rich in small amino acids. The peptide and linker are present on numerous proteins presumed to be targeted to the interiors of 11 of the 15 types of BMCs; for the remaining 4 types of BMCs, the identity of the encapsulated proteins remains unknown, however a subset of these proteins are expected to contain a similar peptide for targeting.

The similarity among peptides targeted to distinct bacterial types implies that the recognition site for the BMC targeting region is located on the BMC shell rather than on other encapsulated components of the BMCs, because the latter vary among BMC type. Sequence comparison indicates that the most strongly conserved positions among the more 2000 BMC shell proteins currently in the database are found at the edges of the shell proteins.

In vitro pull-down assays for interaction used the region found on the C-terminus of the CcmN gene as an isolated peptide (SEQ ID NO:1). The results indicated that the peptide interacted with shell proteins and the CA homolog, CcmM. Fusion of the peptide of SEQ ID NO:1 to YFP appears to result in targeting of the YFP to the carboxysome shell in the cyanobacterium Synechococcus PCC7942 (data not shown).

Thus the region of primary structure (the peptide) appears to be a universal targeting signal for BMCs (and is herein referred to as the “BMC targeting region”).

The secondary structure of the region is predicted to be a single alpha helix flanked on one or both sides by regions predicted to be coil. Most of the predicted alpha helices, which are observed in very different encapsulated proteins, are also predicted to be amphipathic; the helices tend to be characterized by a four (4) residue hydrophobic polar face (positions 10, 6, 9 and 13 in SEQ ID NO:45) opposite a polar face. The conservation of amino acid properties, but lack of absolute sequence identity at each position in the peptide among the targeting/localization regions likely arises from the variability in the amino acid sidechain properties of their cognate shell protein binding partners. However for a given peptide type (e.g. PduP or CcmN) the sequence conservation is strong.

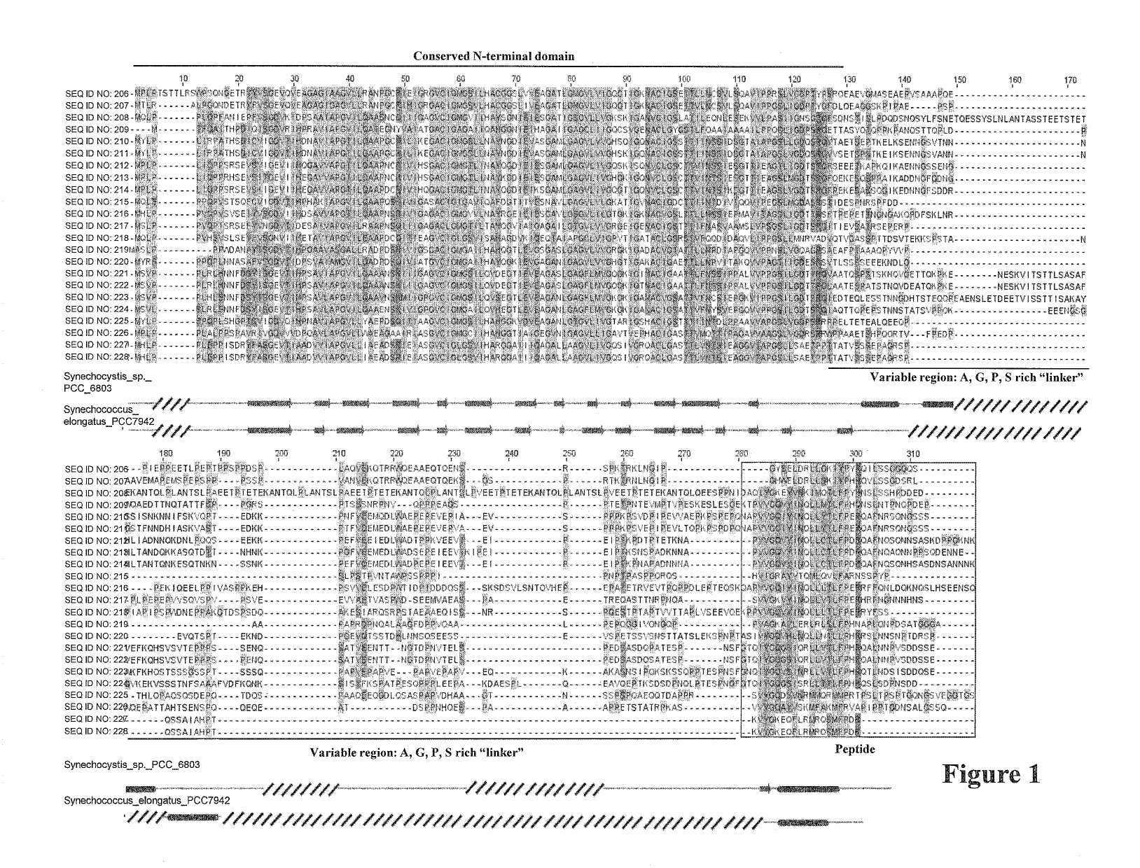

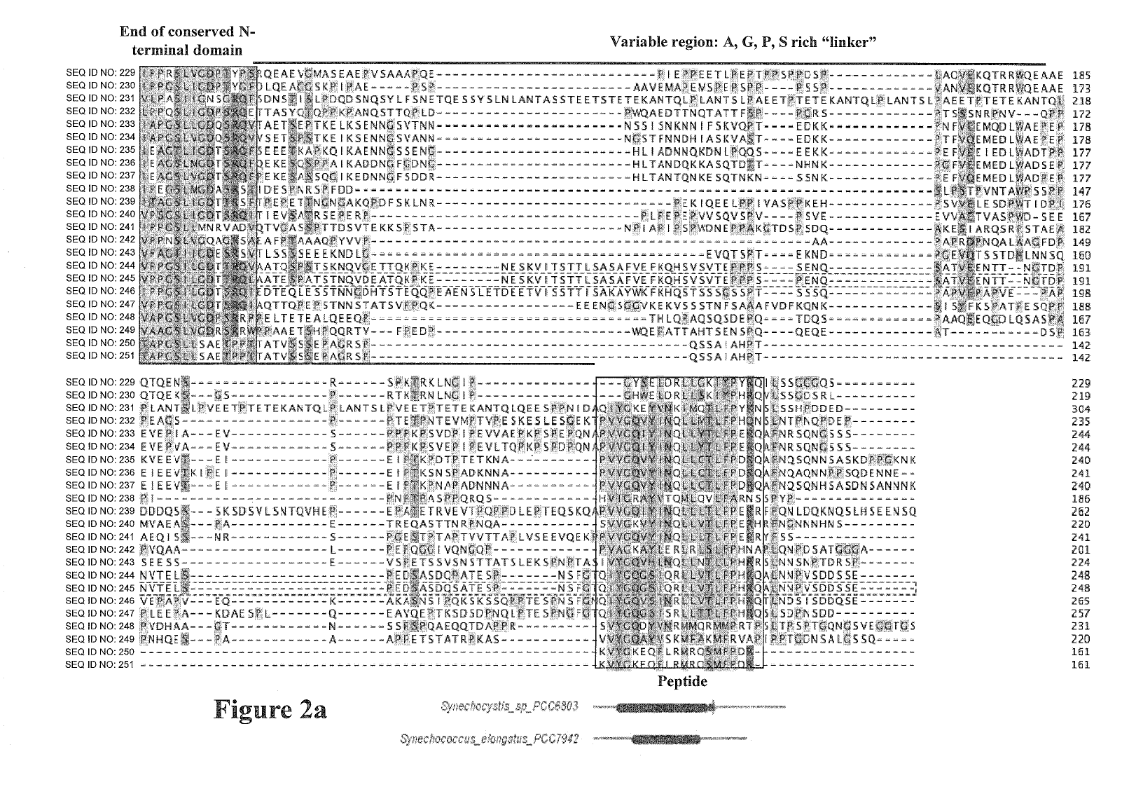

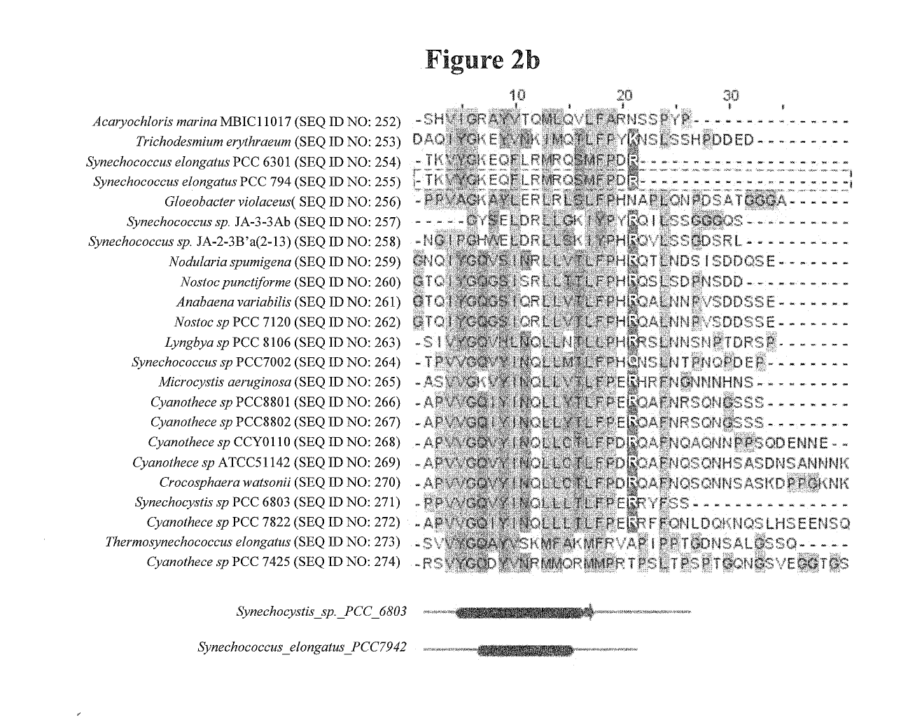

Irrespective of its location in the polypeptide chain, the targeting peptide region is always adjacent to poorly conserved region of amino acids that is rich in proline, glycine, and alanine (the linker region). If the targeting region is located at the N-terminus of an encapsulated protein, it is followed by the linker region and subsequently the functional domain(s) of the protein (See FIGS. 1, 2, 3, 5, 6, 8, 10 and 11). If the region is located on the C-terminus of an encapsulated protein, the functional domain of the protein, followed by the linker precedes it (FIG. 1). If the region is in the middle of a protein encapsulated in a BMC it is flanked on both sides by linker regions (FIG. 10).

All BMC targeting regions share general properties (predicted alpha helical conformation, adjacent to poorly conserved segment(s) of primary structure); for each type of encapsulated protein, for each functionally distinct BMC, we have also identified a consensus amino acid sequence for the targeting region specific to that BMC (Tables 1-3).

Thus, in one embodiment, a common motif found in a subset of proteins presumed to be encapsulated in functionally diverse bacterial microcompartments (BMCs). In another embodiment, targeting peptides which share general properties (predicted alpha helical conformation, flanked by poorly conserved segment(s) of primary structure); for each type of encapsulated protein, for various identified functionally distinct BMC proteins, an identified consensus amino acid sequence for the targeting peptide specific to each of the identified BMCs.

### Definitions

The term “amphipathic alpha helix” or “amphipathic a helix” refers to a polypeptide sequence that can adopt a secondary structure that is helical with one surface, i.e., face, being polar and comprised primarily of hydrophilic amino acids (e.g., Asp, Glu, Lys, Arg, His, Gly, Ser, Thr, Cys, Tyr, Asn and Gln), and the other surface being a nonpolar face that comprises primarily hydrophobic amino acids (e.g., Leu, Ala, Val, Ile, Pro, Phe, Trp and Met) (see, e.g., Kaiser and Kezdy, Ann. Rev. Biophys. Biophys. Chem. 16: 561 (1987), and Science 223:249 (1984)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid polymers may comprise entirely L-amino acids, entirely D-amino acids, or a mixture of L and D amino acids. The use of the term “peptide or peptidomimetic” in the current application merely emphasizes that peptides comprising naturally occurring amino acids as well as modified amino acids are contemplated

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel.

The terms “identical” or percent “identity,” in the context of two or more polypeptide sequences (or two or more nucleic acids), refer to two or more sequences or subsequences that are the same or have a specified percentage of amino acid residues or nucleotides that are the same e.g., 60% identity, preferably 65%, 70%, 75%, 80%, 85%, 90%, 91%, 92%, 93%, 94%, 95%, 96%, 97%, 98%, 99% or 100% identity over a specified region (such as the first 15 out of the 18 amino acids of SEQ ID NO:1), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are typically used.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, polypeptide-nucleic acids (PNAs). Unless otherwise indicated, a particular nucleic acid sequence also encompasses “conservatively modified variants” thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem., 260:2605-2608 (1985); Rossolini et al., Mol. Cell. Probes, 8:91-98 (1994)). The term nucleic acid can be used interchangeably with gene, cDNA, mRNA, oligonucleotide, and polynucleotide.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

A “label” or “detectable label” is a composition detectable by spectroscopic, photochemical, biochemical, immunochemical, or chemical means. For example, useful labels include radioisotopes (e.g., ³H, ³⁵S, ³²P ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available (e.g., the polypeptide such as SEQ ID NOS: 1 or 2 can be made detectable, e.g., by incorporating a radiolabel into the polypeptide, and used to detect antibodies specifically reactive with the polypeptide).

### DESCRIPTIONS OF THE EMBODIMENTS

It will be readily understood by those of skill in the art that the foregoing polypeptides are not fully inclusive of the family of polypeptides of the present invention. In fact, using the teachings provided herein, other suitable polypeptides (e.g., conservative variants) can be routinely produced by, for example, conservative or semi-conservative substitutions (e.g., Asp (D) replaced by Glu (E)), extensions, deletions and the like. In addition, it is contemplated that using the motif described, other suitable polypeptides can be found and screened for desired targeting activities.

Regarding amphipathic α-helix peptides, hydrophobic amino acids are concentrated on one side of the helix, usually with polar or charged amino acids on the other. Different amino-acid sequences have different propensities for forming α-helical structure. Methionine, alanine, leucine, glutamate, and lysine all have especially high helix-forming propensities, whereas proline, glycine, tyrosine, and serine have relatively poor helix-forming propensities. Proline tends to break or kink helices because it cannot donate an amide hydrogen bond (having no amide hydrogen), and because its side chain interferes sterically. Its ring structure also restricts its backbone dihedral angle to the vicinity of −70°, which is less common in α-helices. One of skill understands that although proline may be present at certain positions in the sequences described herein, e.g., at certain positions in the sequence of SEQ ID NO:10 or 31, the presence of more than three prolines within the sequence would be expected to disrupt the helical structure. Accordingly, the polypeptides of the invention do not have more than three prolines, and commonly do not have more than two prolines, present at positions in the alpha-helix forming sequence.

In the presently described peptides and motif, hydrophobic amino acids are considered primarily to include amino acid residues, such as Ile (I), Leu (L), Val (V), Met (M), Phe (F), Tyr (Y), Ala (A), Trp (W). Polar uncharged amino acids are considered primarily to include amino acids such as Gln (Q), Asn (N), Thr (T), Ser (S), and Cys (C). Charged amino acids are considered primarily to include amino acids such as Asp (D), Glu (E), Arg (R), Lys (K), and His (H). When the polar uncharged residues out numbered the charged residues the amino acid property assigned was polar. Proline and glycine are considered neutral amino acids and are not assigned to a specific group.

Thus, in one embodiment, the present invention provides an isolated polypeptide comprising an amino acid sequence in the N-terminal or C-terminal region or inter-domain region of an enzyme in a BMC-associated metabolic pathway in a microorganism comprising the peptides of SEQ ID NOS: 1-192. Table 1 shows the BMC-associated pathway, and the protein and organisms where the peptide is used natively. Also shown is the GenBank Accession number of the protein and the confidence level of the functional prediction of the peptide. Also shown are four organisms and/or metabolic pathways where a conserved region for a peptide may be found using the description of the region as described herein. Each of the GenBank Accessions are hereby incorporated by reference.

TABLE 1

| | Confidence | | | | |
|---|---|---|---|---|---|
| BMC-associated | Level of | | Peptide-containing | | SEQ |
| metabolic | Functional | Representative | ORFs with Locus | Accession | ID |
| pathway | Prediction | organism | Tag | Number | NO: |
| 1 | High (exp) | Synechococcus elongatus | CcmN Cterm | YP_400441 | 1 |
| Calvin cycle | | PCC7942 | (Synpcc7942_1424) | | |
| 1. | High (exp) | Synechococcus elongatus | CcaA | YP_400464 | 2 |
| Calvin cycle | | PCC7942 | (Synpcc7942_1447) | | |
| 2. Ethanolamine | High (exp) | Salmonella typhimurium | EutC Nterm | NP_461392 | 3 |
| utilization | | LT2 (Proteobacteria) | (STM2457) | | |
| | | Clostridium | | | |
| | | phytofermentans ISDg | | | |
| | | (Firmicutes) | | | |
| 2. | High (exp) | Salmonella typhimurium | EutE Nterm | NP_461398 | 4 |
| Ethanolamine | | LT2 (Proteobacteria) | (STM2463) | | |
| utilization | | Clostridium | | | |
| | | phytofermentans ISDg | | | |
| | | (Firmicutes) | | | |
| 2. | High (exp) | Salmonella typhimurium | EutE (Cphy_2642) | YP_001559742 | 5 |
| Ethanolamine | | LT2 (Proteobacteria) | | | |
| utilization | | Clostridium | | | |
| | | phytofermentans ISDg | | | |
| | | (Firmicutes) | | | |
| 3. | High (exp) | Salmonella typhimurium | PduD Nterm | NP_460986 | 6 |
| Propanediol | | LT2 | (STM2041) | | |
| utilization (B12 | | | | | |
| dependent) | | | | | |
| 3. Propanediol | High (exp) | Salmonella typhimurium | PduE Nterm | NP_460987 | 7 |
| utilization (B12 | | LT2 | (STM2042) | | |
| dependent) | | | | | |
| 3. Propanediol | High (exp) | Salmonella typhimurium | PduP Nterm | NP_460996 | 8 |
| utilization (B12 | | LT2 | (STM2051) | | |
| dependent) | | | | | |
| 4. | High (pred) | Rhodopseudomonas | Putative B12- | YP_531045 | 9 |
| 1,2-propanediol | | palustris BisB18 | independent | | |
| utilization (B12 | | | propanediol | | |
| independent) | | | dehydratase | | |
| (putative) | | | (RPC_1163) | | |
| 4. | High (pred) | Rhodopseudomonas | Aldehyde | YP_531056 | 10 |
| 1,2-propanediol | | palustris BisB18 | dehydrogenase | | |
| utilization (B12 | | | Nterm (RPC_1174) | | |
| independent) | | | | | |
| (putative) | | | | | |
| 5. Dissimilation of | High (exp) | Clostridium | Putative B12- | YP_001558291 | 11 |
| fucose and | | phytofermentans ISDg | independeent | | |
| rhamnose to | | | propanediol | | |
| primary alcohols | | | dehydratase | | |
| (putative) | | | (Cphy_1174) | | |
| 5. | High (exp) | Clostridium | Fuculose-phosphate | YP_001558294 | 12 |
| Dissimilation of | | phytofermentans ISDg | aldolase Cterm | | |
| fucose and | | | (Cphy_1177) | | |
| rhamnose to | | | | | |
| primary alcohols | | | | | |
| (putative) | | | | | |
| 5. | High (exp) | Clostridium | Aldehyde | YP_001558295 | 13 |
| Dissimilation of | | phytofermentans ISDg | dehydrogenase | | |
| fucose and | | | (Cphy_1178) Nterm | | |
| rhamnose to | | | | | |
| primary alcohols | | | | | |
| (putative) | | | | | |
| 6. | High (exp) | Clostridium kluyveri DSM | Aldehyde | YP_001394464 | 14 |
| Ethanol utilization | | 555 | dehydrogenases | YP_001394466 | |
| | | | Cterm (Ckl_1074) | | |
| | | | (Ckl_1076) | | |
| 7. | Medium | Planctomyces limnophilus | Aldolase Cterm | | 15 |
| Fuculose-1- | (pred) | DSM 3776 | (Plim_1747) | | |
| phosphate | | | | | |
| metabolism | | | | | |
| (putative) | | | | | |
| 7. | Medium | Planctomyces limnophilus | Aldehyde | | 16 |
| Fuculose-1- | (pred) | DSM 3776 | dehydrogenase | | |
| phosphate | | | Nterm (Plim_1751) | | |
| metabolism | | | | | |
| (putative) | | | | | |
| 8. | Medium | Opitutus terrae PB90-1 | Aldolase Cterm | YP_001818183 | 17 |
| Fuculose-1- | (pred) | | (Oter_1298) | | |
| phosphate and | | | | | |
| rhamnulose-1- | | | | | |
| phosphate | | | | | |
| conversion to | | | | | |
| acetate or pyruvate | | | | | |
| (putative) | | | | | |
| 8. | Medium | Opitutus terrae PB90-1 | Aldehyde | YP_001818180 | 18 |
| Fuculose-1- | (pred) | | dehydrogenase | | |
| phosphate and | | | (Oter_1295) | | |
| rhamnulose-1- | | | | | |
| phosphate | | | | | |
| conversion to | | | | | |
| acetate or pyruvate | | | | | |
| (putative) | | | | | |
| 9. | Medium | Clostridium | Aldehyde | YP_001558530 | 19 |
| Unknown glycyl | (pred) | phytofermentans ISDg | dehydrogenase I | YP_001558542 | |
| radical enzyme | | | (Cphy_1416) Cterm | | |
| (putative) | | | Aldehyde | | |
| | | | dehydrogenase II | | |
| | | | (Cphy_1428) Cterm | | |
| 9. | Medium | Clostridium | unknown glycyl | YP_001558531 | 20 |
| Unknown glycyl | (pred) | phytofermentans ISDg | radical enzyme Nterm | | |
| radical enzyme | | | (Cphy_1417) | | |
| (putative) | | | | | |
| 10. | Med (pred) | Mycobacterium | Aldehyde | YP_884691 | 21 |
| Amino alcohol | Urano et al., | smegmatis MC2 155 | dehydrogenase | | |
| metabolism | 2011 | | Cterm | | |
| (putative) | | | (MSMEG_0276) | | |
| 11. | Low (pred) | Haliangium ochraceum | Aldehyde | ZP_03875711 | 22 |
| Serine-threonine | | SMP-2 | dehydrogenase | | |
| metabolism | | | Nterm | | |
| (putative) | | | (HochDRAFT_00990) | | |
| 12. | Medium | Bacteroides capillosus | unknown | unknown | |
| Glutamate-arginine | (pred) | ATCC 29799 | | | |
| metabolism | | | | | |
| (putative) | | | | | |
| 13. | Low (pred) | Alkaliphilus | unknown | Unknown | |
| Anaerobic purine | | metalliredigens QYMF | | | |
| metabolism | | | | | |
| (putative) | | | | | |
| 14 | Low (pred) | Methylibium | unknown | Unknown | |
| Unknown | | petroleiphilum PM1 | | | |
| 15 | Zero | Chloroherpeton | unknown | Unknown | |
| Unknown | | thalassium ATCC 35110 | | | |

Table 2 shows the actual isolated peptide sequences from the localization region found in the proxy organisms. The BMC associated metabolic pathway is predicted based on experimental evidence and the annotation (using the Integrated Microbial Genomes database found at the Joint Genomes Institute website) of gene products clustered with BMC shell protein genes on the chromosome.

TABLE 2

| | | | Actual ORF peptide |
|---|---|---|---|
| | | | sequence from |
| | | | proxy organism |
| | | | (BOLD = well |
| | | | predicted helical |
| | | | portion; italics = |
| Peptide-containing ORFs | Accession | SEQ | lower confidence in |
| with Locus Tag | Number | ID NO: | predicted helical portion) |
| CcmN Cterm | YP_400441 | 1 | VYGKEQFLRMRQSMFPDR |
| (Synpcc7942_1424) | | | |
| CcaA (Synpcc7942_1447) | YP_400464 | 2 | LAPEQQQRIYRGN |
| EutC Nterm (STM2457) | NP_461392 | 3 | MDQKQIEEIVRSVMAS |
| EutE Nterm (STM2463) | NP_461398 | 4 | MNQQDIEQVVKAVLLKM |
| EutE (Cphy_2642) | YP_001559742 | 5 | NTELVEEIVKRIMKQL |
| PduD Nterm (STM2041) | NP_460986 | 6 | MEINEKLLRQIIEDVLRDM |
| PduE Nterm (STM2042) | NP_460987 | 7 | MNTDAIESMVRDVLSRMNS |
| PduP Nterm (STM2051) | NP_460996 | 8 | MNTSELETLIRTILSE |
| Putative B12 independent | YP_531045 | 9 | AGTNYTEEQVFAAVKKVLNSSGSTDV |
| propanediol dehydratase | | | |
| inter-domain (RPC_1163) | | | |
| Aldehyde dehydrogenase | YP_531056 | 10 | MVAKAIRDHAGTAQPSGNA |
| Nterm (RPC_1174) | | | |
| Putative B12-independeent | YP_001558291 | 11 | IDIILAQQITVQIVKELKERG |
| propanediol dehydratase | | | |
| inter-domain (Cphy_1174) | | | |
| Fuculose-phosphate | YP_001558294 | 12 | DNADLVASITRKVMEQLG |
| aldolase Cterm | | | |
| (Cphy_1177) | | | |
| Aldehyde dehydrogenase | YP_001558295 | 13 | VNEQLVQDIIKNVVASMQLT |
| (Cphy_1178) Nterm | | | |
| Aldehyde dehydrogenases | YP_001394464 | 14 | EPEDNEDVQAIVKAIMAKLNL |
| Cterm(Ckl_1074) | YP_001394466 | | |
| (Ckl_1076) | | | |
| Aldolase Cterm | | 15 | DTEMLVKMITEQVMAALKK |
| (Plim_1747) | | | |
| Aldehyde dehydrogenase | | 16 | MQATEQAIRQVVQEVLAQLN |
| Nterm (Plim_1751) | | | |
| Aldolase Cterm | YP_001818183 | 17 | EVEALVQRLTEEILRQLQ |
| (Oter_1298) | | | |
| Aldehyde dehydrogenase | YP_001818180 | 18 | IDETLVRSVVEEVVRAF |
| (Oter_1295) | | | |
| Aldehyde dehydrogenase I | YP_001558530 | 19 | EDARDLLKQILQALS |
| (Cphy_1416) Cterm | | | |
| Aldehyde dehydrogenase II | YP_001558542 | | |
| (Cphy_1428) Cterm | | | |
| Unknown glycyl radical | YP_001558531 | 20 | MDIREFSNKFVEATKNM |
| enzyme Nterm (Cphy_1417) | | | |
| Aldehyde dehydrogenase | YP_884691 | 21 | LDALRAELRALVVEELAQLIKR |
| Cterm (MSMEG_0276) | | | |
| Aldehyde dehydrogenase | ZP_03875711 | 22 | MALREDRIAEIVERVLARL |
| Nterm (HochDRAFT_00990) | | | |

In another embodiment, consensus peptides SEQ ID NOS: 23-45 are provided for specific BMC-associated pathway enzymes and proteins as shown in Table 3. The residues in parentheses and separated by slashes in the consensus peptides represent that the amino acid at that residue position in the peptide can be chosen from any of the amino acids shown in the parenthesis.

TABLE 3

| | | Metabolic group |
|---|---|---|
| BMC-associated | SEQ | peptide consensus |
| metabolic pathway | ID NO: | (from alignment) |
| Calvin cycle | 23 | (V/I)(V/Y)G(Q/K) |
| | | (V/A/G/E)(Y/S/Q) |
| | | (I/V/L/F)(N/Q/S/L) |
| | | (K/Q/R)(M/L) |
| | | (L/M/R)(V/L/C/Q) |
| | | (T/S)(L/M)FP |
| | | (H/D/E)(R/N/Q) |
| Calvin cycle | 24 | (L/F)(S/P/A)(P/V) |
| | | (E/Q)Q(A/S/Q/W) |
| | | (Q/E/R)RIY(R/Q)G |
| | | (S/N) |
| Ethanolamine | 25 | M(D/N)(E/Q)(K/Q) |
| utilization | | (Q/E)(L/I)(K/R/E) |
| | | (E/D)(I/M)(V/I) |
| | | (R/E)(S/Q)(V/I) |
| | | (L/M)A(E/Q/S) |
| Ethanolamine | 26 | MNQQDIEQVVKAVLLKM |
| utilization | | |
| Ethanolamine | 27 | (A/K/S)(E/D)(A/E) |
| utilization | | L(I/V)(E/D/N) |
| | | (L/E/S)(I/L)(V/I) |
| | | (R/K/E/Q)(K/R)VL |
| | | (E/A)(E/K)L |
| Propanediol | 28 | MEI(N/D/T)E(K/E) |
| utilization | | (L/V)(L/V)(R/E)Q |
| (B12 dependent) | | (I/V)(I/V)(E/K/A) |
| | | (D/E)VL(K/S/R/A) |
| | | (E/D)(M/L) |
| Propanediol | 29 | (M/I)(N/D)(T/E) |
| utilization | | (D/K)(A/L)(I/L)E |
| (B12 dependent) | | (S/E)(M/I)V(R/K) |
| | | (D/E/Q)VL(S,N) |
| | | (M/L)(N/E/G)S |
| Propanediol | 30 | M(N/D/E)(T/S/E) |
| utilization | | (S/L)E(L/V)E |
| (B12 dependent) | | (T/Q/K/D)(L/I) |
| | | (I/V)(R/K)(T/N/K) |
| | | (I/V)(L/I) |
| | | (S/L/R/N)E |
| 1,2-propanediol | 31 | (A/P)(K/G)(S/Q) |
| utilization (B12 | | (S/D)(L/A)(T/N)E |
| independent) | | (E/Q)(D/Q)(I/V)Y |
| (putative) | | (D/E)AVK(K/R) |
| | | (V/I)(L/I)(E/G) |
| | | (Q/E/S)(H/S)G |
| | | (A/S)LD(P/V) |
| 1,2-propanediol | 32 | MN(D/T)(I/T)(E/Q) |
| utilization (B12 | | (I/L)(A/E)(Q/N) |
| independent) | | (A/M)(V/I)(S/R/A) |
| (putative) | | (T/K/N)IL |
| | | (S/A/E/R)(D/K) |
| | | (N/F/Y)(T/L/G)K |
| Dissimilation of | 33 | LD(A/E)ES(A/V) |
| fucose and rhamnose | | (A/G)D(M/I)(T/A) |
| to primary alcohols | | E(M/Q)I(A/L)K |
| (putative) | | (E/G)(L/M)(K/Q) |
| | | (E/D)AG |
| Dissimilation of | 34 | (D/P)(D/N)(A/E) |
| fucose and rhamnose | | (D/E/A)L(V/I)A |
| to primary alcohols | | (E/A/S)IT(K/R) |
| (putative) | | (K/R/Q)V(M/L) |
| | | (A/E)QL(G/K) |
| Dissimilation of | 35 | VNEQ(L/M)VQDIV |
| fucose and rhamnose | | (Q/R/K)EVVA(K/R) |
| to primary alcohols | | MQI(S/T) |
| (putative) | | |
| Ethanol utilization | 36 | EPEDNEDVQAIVKAIM |
| | | AKLNL Aldehyde |
| | | dehydrogenase |
| | | Cterm- unique as |
| | | a group but |
| | | similar to other |
| | | Cterm Aldehyde |
| | | dehydrogenase |
| | | tags |
| Fuculose-1- | 37 | DQE(A/Q)LV(K/Q) |
| phosphate | | (A/L)IT(D/E) |
| metabolism (putative) | | (Q/R/E)VMA(A/E) |
| | | L(K/S)K |
| Fuculose-1- | 38 | MQ(I/A)(D/T)EE |
| phosphate | | (L/A)IRSVV(A/Q) |
| metabolism (putative) | | (Q/E)VL(A/S) |
| | | (E/Q)(V/L)(G/N) |
| Fuculose-1- | 39 | EVEALVQRLTEEILRQ |
| phosphate and | | LQ Aldolase |
| rhamnulose-1- | | Cterm- unique as |
| phosphate conversion | | a group but |
| to acetate or | | similar to other |
| pyruvate (putative) | | Cterm aldolase |
| | | tags |
| Fuculose-1- | 40 | IDETLVRSWEEWRAF |
| phosphate and | | Aldehyde |
| rhamnulose-1- | | dehydrogenase |
| phosphate conversion | | Nterm- unique as |
| to acetate or | | a group but |
| pyruvate (putative) | | similar to other |
| | | Nterm Aldehyde |
| | | dehydrogenase |
| | | tags |
| Unknown glycyl | 41 | (E/Q/D)(N/E/D) |
| radical enzyme | | (V/I/L)(E/Q/A) |
| (putative) | | (R/Q/D)(I/L/V) |
| | | (I/L/V)(K/R/N) |
| | | (E/Q/K)(V/I/L) |
| | | (L/I/V)(E/Q/G) |
| | | (Q/R/A)(L/M) |
| | | (K/G/S) |
| Unknown glycyl | 42 | M(A/D)(K/I/N/L) |
| radical enzyme | | (R/Y/)(E/N/S/) |
| (putative) | | (L/F)(T/S)(P/N) |
| | | (R/K)(V/L/F)(K/A) |
| | | (E/V/M)(L/A)(A/T) |
| | | (E/K)(R/N)(L/M) |
| Arginine or | 43 | I(E/D/G)ALR |
| serine/threonine | | (A/E/D)ELR(A/R)L |
| metabolism (putative) | | (V/I)(V/A)EEL |
| | | (A/R)(Q/E)L |
| | | (I/N/G)(K/R)(R/Q) |
| Serine-threonine | 44 | MALREDRIAEIVERVLA |
| metabolism (putative) | | RL unique as |
| | | group but similar |
| | | to other Nterm |
| | | Aldehyde |
| | | dehydrogenase |
| | | tags |

Table 4 is a compilation of Tables 1-3 plus additional notes and information.

TABLE 4

| | | | | | Actual ORF peptide | |
|---|---|---|---|---|---|---|
| | | | | | sequence from | |
| | | | | | proxy organism | |
| | | | | Peptide- | (BOLD = well | |
| | | | | containing | predicted | |
| | BMC- | Confidence | | ORFs with Locus | helical portion. | |
| | associated | Level of | | Tag and | RED = not | Metabolic group |
| Group | metabolic | Functional | Representative | Accession | well predicted | peptide consensus |
| # | pathway | Prediction | organism | Number | helical portion) | (from alignment) |
| 1 | Calvin cycle | High | Synechococcus | CcmN | CcmN Cterm- | CcmN Cterm- |
| | | (exp) | elongatus | (Synpcc7942_1424) | VYGKEQFLRMRQSMFPDR | (V/I)(V/Y)G(Q/K)(V/A/G/E)(Y/S/Q)(I/V/L/F) |
| | | | PCC7942 | YP_400441 | CcaA Cterm- | (N/Q/S/L)(K/Q/R)(M/L)(L/M/R)(V/L/C/Q) |
| | | | | CcaA | LAPEQQQRIYRGN | (T/S)(L/M)FP(H/D/E)(R/N/Q) |
| | | | | (Synpcc7942_1447) | | CcaA Cterm- |
| | | | | YP_400464 | | (L/F)(S/P/A)(P/V)(E/Q)Q(A/S/Q/W)(Q/E/R) |
| | | | | | | RIY(R/Q)G(S/N) |
| 2 | Ethanolamine | High | Salmonella | EutC (STM2457) | EutC Nterm- | EutC Nterm (firmicute/proteobacteria) |
| | utilization | (exp) | typhimurium | NP_461392 | MDQKQIEEIVRSVMAS | M(D/N)(E/Q)(K/Q)(Q/E)(L/I)(K/R/E)(E/D) |
| | | | LT2 | EutE (STM2463) | EutE Nterm | (I/M)(V/I)(R/E)(S/Q)(V/I)(L/M)A(E/Q/S) |
| | | | (Proteobacteria) | NP_461398 | (Proteobacteria)- | EutE Nterm (Proteobacteria)- |
| | | | Clostridium | EutE (Cphy_1542) | MNQQDIEQVVKAVLLKM | MNQQDIEQVVKAVLLKM |
| | | | phytofermentans | YP_001559742 | EutE Cterm | EutE Cterm (Firmicutes)- |
| | | | ISDg | | (Firmicutes)- | (A/K/S)(E/D)(A/E)L(I/V)(E/D/N)(L/E/S) |
| | | | (Firmicutes) | | NTELVEEIVKRIMKQL | (I/L)(V/I)(R/K/E/Q)(K/R)VL(E/A)(E/K)L |
| 3 | Propanediol | High | Salmonella | PduD (STM2041) | PduD Nterm- | PduD Nterm- |
| | utilization | (exp) | typhimurium_T2 | NP_460986 | MEINEKLLRQIIEDVLRDM | MEI(N/D/T)E(K/E)(L/V)(L/V)(R/E)Q(I/V) |
| | (B12 | | | PduE (STM2042) | PduE Nterm- | (I/V)(E/K/A)(D/E)VL(K/S/R/A)(E/D)(M/L) |
| | dependent) | | | NP_460987 | MNTDAIESMVRDVLSRMNS | PduE Nterm- |
| | | | | PduP (STM2051) | PduP Nterm- | (M/I)(N/D)(T/E)(D/K)(A/L)(I/L)E(S/E)(M/I) |
| | | | | NP_460996 | MNTSELETLIRTILSE | V(R/K)(D/E/Q)VL(S/N)(M/L)(N/E/G)S |
| | | | | | | PduP Nterm- |
| | | | | | | M(N/D/E)(T/S/E)(S/L)E(L/V)E(T/Q/K/D)(L/I) |
| | | | | | | (I/V)(R/K)(T/N/K)(I/V)(L/I)(S/L/R/N)E |
| 4 | 1,2- | High | Rhodopseudomonas | Putative | Pdu Interdomain | Pdu (B12-independent)- |
| | propanediol | (pred) | palustris BisB18 | B12-independent | linker- | (A/P)(K/G)(S/Q)(S/D)(L/A)(T/N)E(E/Q)(D/Q) |
| | utilization | | | propanediol | AGTNYTEEQVFAAVKKVLNSSGSTDV | (I/V)Y(D/E)AVK(K/R)(V/I)(L/I)(E/G)(Q/E/S) |
| | (B12 | | | dehydratase | Aldehyde dehydrogenase | (H/S)G(A/S)LD(P/V) |
| | independent) | | | (RPC_1163) | Nterm- | Aldehyde dehydrogenase Nterm- |
| | (putative) | | | YP_531045 | MVAKAIRDHAGTAQPSGNA | MN(D/T)(I/T)(E/Q)(I/L)(A/E)(Q/N)(A/M) |
| | | | | Aldehyde | | (V/I)(S/R/A)(T/K/N)IL(S/A/E/R)(D/K) |
| | | | | dehydrogenase | | (N/F/Y)(T/L/G)K |
| | | | | (RPC_1174) | | |
| | | | | YP_531056 | | |
| 5 | Dissimitation | High | Clostridium | Putative | Cphy_1174 interdomain | Pdu (B12-independent)- |
| | of fucose and | (exp) | phytofermentans | B12-independeent | linker- | EVGE(D/K)EIAA9I/V)LXTVLE(A/M)(E/K)LP |
| | rhamnose to | | ISDg | propanediol | EKEIEQILKTVLEAKKENTE | Fuculose-phosphate aldolase Cterm- |
| | primary | | | dehydratase | Cphy_1177 Cterm- | (D/P)(D/N)(A/E)(D/E/A)L(V/I)A(E/A/S)IT |
| | alcohols | | | (Cphy_1174) | DNADLVASITRKVMEQLG | (K/R)(K/R/Q)V(M/L)(A/E)QL(G/K) |
| | (putative | | | YP_001558291 | Cphy_1178 Nterm- | Aldehyde dehydrogenase N-term- |
| | | | | Fuculose- | VNEQLVQDIIKNVVASMQLT | VNEQ(L/M)VQDIV(Q/R/K)EVVA(K/R)MQ(S/T) |
| | | | | phosphate | | |
| | | | | aldolase | | |
| | | | | (cphy_1177) | | |
| | | | | YP_001558294 | | |
| | | | | Aldehyde | | |
| | | | | dehydrogenase | | |
| | | | | (Cphy_1178) | | |
| | | | | YP_001558295 | | |
| 6 | Ethanol | High | Clostridium | Aldehyde | Aldehyde dehydrogenase | Aldehyde dehydrogenase Cterm- |
| | utilization | (exp) | kluyveri | dehydrogenases | Cterm- | unique as a group but similar to other |
| | | | DSM 555 | (Ckl_1074) | EPEDNEDVQAIVKAIMAKLNL | Cterm Aldehyde dehydrogenase tags |
| | | | | YP_001394464 | | |
| | | | | (Ckl_1076) | | |
| | | | | YP_001394466 | | |
| 7 | Fuculose-1- | Medium | Planctomyces | Aldolase | Plim_1747 Cterm- | Aldolase Cterm- |
| | phosphate | (pred) | limnophilus | (Plim_1747) | DTEMLVKMITEQVMAALKK | DQE(A/Q)LV(K/Q)(A/L)IT(D/E)(Q/R/E)V |
| | metabolism | | DSM 3776 | Aldehyde | Plim_1751 Nterm- | MA(A/E)L(K/S)K |
| | (putative) | | | dehydrogenase | MQATEQAIRQVVQEVLAQLN | Aldehyde dehydrogenase Nterm- |
| | | | | (Plim_1751) | | MQ(I/A)(D/T)EE(L/A)IRSVV(A/Q)(Q/E)V |
| | | | | | | L(A/S)(E/Q)(V/L)(G/N) |
| 8 | Fuculose-1- | Medium | Opitutus terrae | Aldolase | Oter_1298 Cterm- | Aldolase Cterm- unique as a group but |
| | phosphate and | (pred) | PB90-1 | (Oter_1298) | EVEALVQRLTEEILRQLQ | similar to other Cterm aldolase tags |
| | rhamnulose-1- | | | YP_001818183 | Oter_1295 Nterm- | Aldehyde dehydrogenase Nterm- |
| | phosphate | | | Aldehyde | IDETLVRSVVEEVVRAF | unique as a group but similar to other |
| | conversion to | | | dehydrogenase | | Nterm Aldehyde dehydrogenase tags |
| | acetate or | | | (Oter_1295) | | |
| | pyruvate | | | YP_001818180 | | |
| | (putative) | | | | | |
| 9 | Unknown | Medium | Clostridium | Aldehyde | Cphy_1416 Cterm- | Aldehyde dehydrogenase Cterm- |
| | glycyl | (pred) | Phytofermentans | dehydrogenase I | EDARDLLKQILQALS | (E/Q/D)(N/E/D)(V/I/L)(E/Q/A)(R/Q/D) |
| | radical | | ISDg | (Cphy_1416) | Cphy_1417 Nterm- | (I/L/V)(K/R/N)(E/Q/K)(V/I/L)(L/I/V) |
| | enzyme | | | YP_001558530 | MDIREFSNKFVEATKNM | (E/Q/G)(Q/R/A)(L/M)(K/G/S) |
| | (putative) | | | Aldehyde | | Unknown glycyl radical enzyme Nterm- |
| | | | | dehydrogenase II | | M(A/D)(K/I/N/L)(R/Y/)(E/N/S/)(L/F)(T/S) |
| | | | | (Cphy_1428) | | (P/N)(R/K)(V/L/F)(K/A)(E/V/M)(L/A)(A/T) |
| | | | | YP_001558542 | | (E/K)(R/N)(L/M) |
| | | | | unknown glycyl | | |
| | | | | radical enzyme | | |
| | | | | (Cphy_1417) | | |
| | | | | YP_001558531 | | |
| 10 | Arginine or | Low | Mycobacterium | Aldehyde | MSMEG_0276 Cterm- | Aldehyde dehydrogenase Cterm- |
| | serine/ | (pred) | smegmatis | dehydrogenase | LDALRAELRALVVEELAQLIKR | I(E/D/G)ALR(A/E/D)ELR(A/R)L(V/I)(V/A) |
| | threonine | | MC2 155 | (MSMEG_0276) | | EEL(A/R)(Q/E)L(I/N/G)(K/R)(R/Q) |
| | metabolism | | | YP_884691 | | |
| | (putative) | | | | | |
| 11 | Serine- | Low | Haliangium | Aldehyde | HochDRAFT_00990 Nterm- | Aldehyde dehydrogenase Nterm- |
| | threonine | (pred) | ochraceum | dehydrogenase | MALREDRIAEIVERVLARL | unique as a group but similar to other |
| | metabolism | | SMP-2 | (HochDRAFT_00990) | | Nterm Aldehyde dehydrogenase tags |
| | (putative) | | | ZP_03875711 | | |
| 12 | Glutamate- | Medium | Bacteroides | unknown | unknown | unknown |
| | arginine | (pred) | capillosus | | | |
| | metabolism | | ATCC 29799 | | | |
| | (putative) | | | | | |
| 13 | Anaerobic | Low | Alkaliphilus | unknown | unknown | unknown |
| | purine | (pred) | metalliredigens | | | |
| | metabolism | | QYMF | | | |
| | (putative) | | | | | |
| 14 | Unknown | Low | Methylibium | unknown | unknown | unknown |
| | | (pred) | petroleiphilum | | | |
| | | | PM1 | | | |
| 15 | Unknown | Zero | Chloroherpeton | unknown | unknown | unknown |
| | | | thalassium | | | |
| | | | ATCC 35110 | | | |
| | Potentially | | | | | |
| Group | encapsulated | GOID | Organism | | Reason for | |
| # | reactions | range | phenotypes | Enzymes | encapsulation | Additional Notes |
| 1 | Bicarbonate → | 637799853- | Aerobe | Carbonic | RuBisCO | |
| | carbon | 637799857 | | anhydrase, | inefficiency | |
| | dioxide → | | | RuBisCO | RuBisCO oxygen | |
| | glycerate3- | | | | sensativity | |
| | phosphate | | | | product | |
| | | | | | toxicity | |
| 2 | Ethanolamine → | 637213172- | Aerobe | Ethanolamine | Oxygen | |
| | Acetaldehyde → | 637213188 | | ammonia | sensitivity | |
| | Acetyl-CoA | | | lyase (EutBC), | product | |
| | | | | acetaldehyde | volatility/ | |
| | | | | dehydrogenase | toxicity | |
| | | | | (EutE) | | |
| 3 | 1,2- | 637212757- | Aerobe | 1,2- | Oxygen | |
| | propanediol → | 637212777 | | propanediol | sensitivity | |
| | proprionaldehyde → | | | dehydratase | product | |
| | propanol | | | (PduCDE), | volatility/ | |
| | | | | B12-dependent | toxicity | |
| | | | | propionaldehyde | | |
| | | | | dehydrogenase | | |
| | | | | (PduP) | | |
| 4 | 1,2- | 637924274- | Generally | Putative 1,2- | Oxygen | |
| | propanediol → | 637924291 | anaerobic | propanediol | sensitivity | |
| | proprionaldehyde → | | maybe | dehydrogenase, | product | |
| | propanol | | facultative | B12-independent | volatility/ | |
| | | | | (GRE), | toxicity | |
| | | | | propionaldehyde | | |
| | | | | dehydrogenase | | |
| | | | | (PduP) | | |
| 5 | Fuculose-1- | 641292279- | Anaerobe | Putative 1,2- | Product | A fusion of the B12-independent |
| | phosphate → | 641292292 | | propanediol | volatility/ | 1,2-propandiol dehydrogenase and |
| | lactaldehyde → | | | dehydrogenase, | toxicity | fuculose degradation pathways |
| | 1,2- | | | B12-independent | | |
| | propanediol → | | | (GRE), | | |
| | proprionaldehyde → | | | propionaldehyde | | |
| | propanol | | | dehydrogenase | | |
| | | | | (PduP) | | |
| | | | | Fuculose-1- | | |
| | | | | phosphate | | |
| | | | | aldolase | | |
| | | | | lactaldehyde | | |
| | | | | oxidoreductase | | |
| 6 | Ethanol → | 640858318- | Anaerove: Can | Aldehyde | Product | No nearby 03319 genes; Alcohol |
| | Acetaldehyde → | 640858324 | grow on | dehydrogenase; | volatility/ | dehydrogenases are probably |
| | Acetyl-CoA | | ethanol, | alcohol | toxicity | encapsulated from experimental |
| | | | acetate only | dehydrogenase | | evidence, but no obvious peptide like |
| | | | | | | sequence found |
| 7 | Fuculose-1- | 2501576836- | Aerobe | Fuculose-1- | Product | |
| | phosphate → | 2501576848 | | phosphate | volatility/ | |
| | lactaldehyde → | | | aldolase | toxicity | |
| | ? | | | | | |
| 8 | Fuculose-1- | 641690930- | Obligate | Fuculose/ | Product | Nearly identical to the enzymes found |
| | phosphate or | 641690944 | anaerobe | rhamnulose-1- | volatility/ | in Planctomycetes but also includes the |
| | rhamnulose-1- | | | phosphate | toxicity | rhamnulose degradation pathway |
| | phosphate → | | | aldolase; | | |
| | lactaldehyde → | | | aldehyde | | |
| | lactate | | | dehydrogenase | | |
| 9 | Unknown; Highest | 641292513- | Anaerobe | Unknown glycyl | Oxygen | |
| | homology to | 641292533 | | radical with | sensitivity | |
| | glycerol | | | homology to | product | |
| | dehydratase, but | | | glycerol | volatility/ | |
| | not a GD | | | dehydratase | toxicity | |
| 10 | L-aspartate-4- | 639738830- | Aerobe, non | Aldehyde | Product | |
| | semialdehyde or | 639738839 | pathogenic | dehydrogenase; | volatility/ | |
| | glutamate-5- | | | aminotransferase | toxicity | |
| | semialdehyde | | | type III | | |
| | based reactions | | | | | |
| 11 | Homoserine <--> | 644016663- | Aerobe | L-homoserine: | Product | |
| | L-aspartate 4- | 644018672 | | NAD+ | volatility/ | |
| | semialdehyde <--> ? | | | oxidoreductase | toxicity | |
| | | | | (not in BMC, in | | |
| | | | | genome); | | |
| | | | | dihydrodipicolinate | | |
| | | | | synthase or other | | |
| | | | | enzymes that | | |
| | | | | function on | | |
| | | | | L-aspartate-4- | | |
| | | | | semialdehyde (not | | |
| | | | | in BMC; in genome) | | |
| 12 | N-acetyl- | 641050502- | Aerotolerant | N-acetyl- | Product | Contains entire glutamate-arginine |
| | glutamylphosphate → | 641050513 | anaerobe; | gammaglutamyl | volatility/ | conversion pathway; 2 00936 proteins, |
| | N-acetylglutamate | | pathogen | phosphate | toxicity | no nearby 03319s |
| | semialdehyde → | | | reductase, | | |
| | N-acetylornithine | | | acetylornithine | | |
| | | | | aminotransferase | | |
| 13 | Hypoxanthine → | 640785432- | Anaerobe | Xanthine | Xanthine | |
| | xanthine → | 640785453 | | dehydrogenase; | toxicity | |
| | 5-ureido-4- | | | Xanthine hydrolase | | |
| | imidazole | | | | | |
| | carboxylate | | | | | |
| 14 | Unknown | 640092924- | Aerobe | PduP/EutE aldehyde | Product | |
| | aldehyde | 640092931 | | dehydrogenase; | volatility/ | |
| | metabolism | | | putative | toxicity | |
| | | | | glutathione | | |
| | | | | dependent | | |
| | | | | formaldeyde | | |
| | | | | dehydrogenase | | |
| 15 | Unknown | | Anaerobic | No readily apparent | Unknown | 2 pfam00936, 3 pfam03319 scattered |
| | | | photoautotrophic | encapsulated | | throughout genome |
| | | | | enzymes near | | |
| | | | | 00936/03319 | | |
| | | | | proteins | | |

Shown another way, the present invention provides isolated consensus polypeptides in Table 3. For example, SEQ ID NO: 23 comprising:

(SEQ ID NO: 23)
X₁X₂GX₄X₅X₆X₇X₈X₉X₁₀X₁₁X₁₂X₁₃X₁₄FPX₁₇X₁₈

wherein: X₁ is V or I; X₂ is V or Y, X₄ is Q or K, X₅ is V, A, G or E, X₆ is Y, S or Q, X₇ is I, V, L or F, X₈ is N, Q, S or L, X₉ is K, Q or R, X₁₀ is M or L, X₁₁ L, M or R, X₁₂ is V, L, C or Q, X₁₃, is T or 5, X₁₄ is L or M, X₁₇ is H, D or E and X₁₈ is R, N or Q.

Thus shown in another way, SEQ ID NO:25 is:

| | | | | | | | | | | 1 | 1 | 1 | 1 | 1 | 1 | 1 |
|---|---|---|---|---|---|---|---|---|---|---|---|---|---|---|---|---|
| Postn¹ | 1 | 2 | 3 | 4 | 5 | 6 | 7 | 8 | 9 | 0 | 1 | 2 | 3 | 4 | 5 | 6 |
| AA(s) | M | D | E | K | Q | L | K | E | I | V | R | S | V | L | A | E |
| | | N | Q | Q | E | I | R | D | M | I | E | Q | I | M | | Q |
| | | | | | | | E | | | | | | | | | S |

In another embodiment, a targeting peptide is designed based on a consensus motif identified in the targeting peptides. Shown in an analysis of an alignment of all bacterial microcompartment targeting peptides (FIG. 3B), a distillation of the core amino acid properties (i.e. hydrophobic, polar, or charged) at each aligned position of the peptide was made based on the abundance of residues that fall into certain property groups at that position. FIG. 3C shows the amino acid percentage at each of the 17 well-aligned positions in the alignment of 305 unique bacterial microcompartment targeting peptides. Thus a consensus amino acid property can be assigned to each position. In the consensus motif shown in FIG. 3C, majority amino acid percentages at each well-aligned position were calculated in JALVIEW.

Amino acid property at each position in the motif was given based on the majority amino acid property (H=hydrophobic, C=charged, P=polar) at each aligned position. Positions 5 and 15 were highly variable based on identity and property and no consensus property denoted by an X. Thus, the motif can be identified as:

Consensus Motif: H P C C X H C P H H C C H H X C H

where:

H=Hydrophobic Residues (Amino acids I, L, V, M, F, Y, A, W)

P=polar uncharged Residues (Amino acids Q, N, T, S, C)

C=Charged Residues (Amino acids D, E, R, K, H)

X=Any amino acid

Thus in one embodiment, the consensus motif allows one to design a targeting polypeptide. When mapped onto a helical wheel projection determined by a consensus of alpha helical secondary structure predictions of the peptides, one can create a consensus amphipathic helix for targeting bacterial microcompartments. For example, SEQ ID NO: 45 comprising:

(SEQ ID NO: 45)
X₁X₂X₃X₄X₅X₆X₇X₈X₉X₁₀X₁₁X₁₂X₁₃X₁₄X₁₅X₁₆X₁₇

wherein:

X₁ is I, L, V, M, F, Y, A, or W;

X₂ is Q, N, T, S, or C,

X₃ is D, E, R, K, or H,

X₄ is D, E, R, K, or H,

X₅ is any residue,

X₆ is I, L, V, M, F, Y, A, or W,

X₇ is D, E, R, K, or H,

X₈ is Q, N, T, S, or C,

X₉ is I, L, V, M, F, Y, A, or W,

X₁₀ is I, L, V, M, F, Y, A, or W,

X₁₁ is D, E, R, K, or H,

X₁₂ is D, E, R, K, or H,

X₁₃, is I, L, V, M, F, Y, A, or W,

X₁₄ is I, L, V, M, F, Y, A, or W,

X₁₅ is any residue, and

X₁₆ is D, E, R, K, or H, and

X₁₇ is I, L, V, M, F, Y, A, or W.

Thus shown in another way, SEQ ID NO:45 is:

| Postn¹ | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| AA(s) | I | Q | D | D | X | I | D | Q | I | I | D | D | I | I | X | D | I |
| | L | N | E | E | | L | E | N | L | L | E | E | L | L | | E | L |
| | V | T | R | R | | V | R | T | V | V | R | R | V | V | | R | V |
| | M | S | K | K | | M | K | S | M | M | K | K | M | M | | K | M |
| | F | C | H | H | | F | H | C | F | F | H | H | F | F | | H | F |
| | Y | | | | | Y | | | Y | Y | | | Y | Y | | | Y |
| | A | | | | | A | | | A | A | | | A | A | | | A |
| | | | | | | W | | | W | W | | | W | W | | | W |
| | W | | | | | | | | | | | | | | | | |

In another embodiment, using the polypeptides of SEQ ID NOS: 1-192, a mechanism is provided for targeting biological molecules that would benefit from being compartmentalized and/or recombining them with other molecules and biological molecules within a bacterial microcompartment shell. This will enable the engineering of new or enhanced bacterial microcompartments. An example strategy is in one embodiment, a carboxysome shell protein is co-expressed with a fluorescent protein-peptide fusion. These protein-peptide fusions can be transferred among organisms (e.g. bacteria, fungi, plants, algae) using basic molecular techniques, followed by directed evolution to optimize phenotype. Alternatively, the modules are stable in solution or can be engineered to be (e.g., via reversible bonds/crosslinks), stable in solution, thus carrying out catalysis in cell free, non-biological systems.

In another embodiment, this allows one to engineer new metabolic modules (essentially organelles of specific function) into bacteria and it provides a new approach to designing and optimizing catalysis in solution. For example, insertion of polynucleotides encoding for the expression of the peptides provided for in SEQ ID NOS: 1-46, 145-190 or for example, at least the localization peptide regions in the polypeptides of SEQ ID NOS: 47-144 or 194-349.

In one embodiment, a bacterial microcompartment (BMC) and metabolic pathway is selected to be engineered. The polynucleotide encoding the bacterial compartment and enzymes in the metabolic pathway can be inserted into a host organism and if needed, expressed using an inducible expression system. The polynucleotide sequence encoding the peptides of SEQ ID NOS:1-192, 194-349, or a fragment thereof, can be inserted into the protein(s) in the N-terminus or C-terminus or between functional domains of the proteins, thereby permitting the encapsulation of the protein into the BMC upon expression. When referring to the bacterial compartments or microcompartments, it is meant to include any number of proteins, shell proteins or enzymes (e.g., dehydrogenases, aldolases, lyases, etc.) that comprise or are encapsulated in the compartment

In one embodiment, polynucleotides encoding a bacterial microcompartment shell proteins, and proteins containing a localization peptide (SEQ ID NOS: 1-192), are cloned into an appropriate plasmid under an inducible promoter, inserted into vector, and used to transform cells, such as E. coli, cyanobacteria, plants, algae, or other photosynthetic organisms. This system maintains the expression of the inserted gene silent unless an inducer molecule (e.g., IPTG) is added to the medium.

Bacterial colonies are allowed to grow after induction of gene expression. In one embodiment, the presently described peptides described in SEQ ID NOS: 1-192 are contemplated for use in any of the applications herein described.

In another embodiment, an expression vector comprising a nucleic acid sequence for a cluster of bacterial compartment genes and include a polynucleotide sequence which encodes any of the peptides of SEQ ID NOS:1-192 or a fragment thereof, which is then expressed in an organism by addition of an inducer molecule.

In some embodiments, expression cassettes comprising a promoter operably linked to a heterologous nucleotide sequence of the invention, i.e., any nucleotide sequence which encodes for a peptide comprising SEQ ID NOS:1-192 or a fragment thereof, that encodes a localization target sequence for microcompartment RNA or polypeptide are further provided. The expression cassettes of the invention find use in generating transformed plants, plant cells, microorganisms algae, fungi, and other eukaryotic organisms as is known in the art and described herein. The expression cassette will include 5′ and 3′ regulatory sequences operably linked to a polynucleotide of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be co-transformed into the organism. Alternatively, the additional gene(s) can be provided on multiple expression cassettes. Such an expression cassette is provided with a plurality of restriction sites and/or recombination sites for insertion of the polynucleotide that encodes a microcompartment RNA or polypeptide to be under the transcriptional regulation of the regulatory regions. The expression cassette may additionally contain selectable marker genes.

The expression cassette will include in the 5′-3′ direction of transcription, a transcriptional initiation region (i.e., a promoter), translational initiation region, a polynucleotide of the invention, a translational termination region and, optionally, a transcriptional termination region functional in the host organism. The regulatory regions (i.e., promoters, transcriptional regulatory regions, and translational termination regions) and/or the polynucleotide of the invention may be native/analogous to the host cell or to each other. Alternatively, the regulatory regions and/or the polynucleotide of the invention may be heterologous to the host cell or to each other. As used herein, “heterologous” in reference to a sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

Where appropriate, the polynucleotides may be optimized for increased expression in the transformed organism. For example, the polynucleotides can be synthesized using preferred codons for improved expression.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 6:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). The above list of selectable marker genes is not meant to be limiting. Any selectable marker gene can be used in the present invention.

In another embodiment, it may be beneficial to express the gene from an inducible promoter, particularly from an inducible promoter. The gene product may also be co-expressed with a polypeptide comprising SEQ ID NOS: 1-192 or fragment thereof, such that the polypeptide is in the C-terminal or N-terminal region.

In one embodiment, an in-vitro transcription/translation system (e.g., Roche RTS 100 E. coli HY) can be used to produce cell-free microcompartments or expression products which may be targeted by the polypeptides of the current invention.

In some embodiments, it is preferred that the microcompartments, comprising the microcompartment nucleic acids, proteins or polypeptides of the present invention described above, should provide an organism enhanced biomass production and CO₂ sequestration abilities, or produce valuable intermediates (Acetyl CoA), or sequester and protect oxygen-sensitive enzymes (engineered or native) or encapsulate reactions that would otherwise be toxic to the cell but however, be non-toxic or have low toxicity levels to humans, animals and plants or other organisms that are not the target.

The microcompartment proteins are preferably incorporated into a microorganism or eukaryote (plant, algae, yeast/fungi) to provide new or enhanced metabolic activity. In some embodiments, the microcompartment proteins are incorporated to provide enhanced carbon fixation and sequestration activity in the plant or organism (i.e., addition of a carboxysome) or produce valuable intermediates (Acetyl CoA), or sequester and protect oxygen-sensitive enzymes (engineered or native) or encapsulate reactions that would otherwise be toxic to the cell.

In another embodiment, a peptide of SEQ ID NO: 1-192 or fragment thereof, is used to target a biomolecule to a surface or a substrate. The peptides, which are derived from the targeting region of native BMC proteins and enzymes, appear to target the hexameric facets of BMC shell proteins. The biomolecule can be any native or modified protein, enzyme, cofactor, polymer, polysaccharide, polypeptide, or other biomolecule.

In another embodiment, when a surface comprising a BMC shell protein is made in vivo or in vitro, a peptide of SEQ ID NO:1-192 or fragment thereof, can be attached to a molecule or material whereby the peptide will localize the molecule or material to the surface of this molecular layer. It is contemplated that peptides SEQ ID NOS:1-192 or fragment thereof, can be used to tether any molecule or material to a substrate comprising a BMC shell protein. The substrate can be any shape or surface, such as a flat surface or molecular scaffold.

### EXAMPLE 1

### Identification of Consensus Sequence and Secondary Structure Prediction of Conserved C-Termini in Carboxysomal Protein, CcmN

Carboxysome protein, CcmN, and its orthologues from all β-cyanobacterial species were aligned and compared using MUSCLE (Edgar et al. (2004) Nucleic Acids Research 32: 1792-97). For example, when visualized using Jalview (Waterhouse and Procter et al. (2009) Bioinformatics 25: 1189-91), the consensus function built into the program produces SEQ ID NO:46, where the black bars represent percent identity.

The CcmN amino acid sequences from two of the most well studied β-cyanobacterial species, Synechocystis sp. PCC6803 and Synechococcus elongatus PCC7942, were analyzed using the Jpred 3 server (Cole et al. (2008) Nucleic Acids Research 36: W197-W201), to determine the predicted secondary structure of the conserved C-termini of the proteins. The secondary structures for each protein are shown below, where the gray line represents a coil or loop motif, the black bar represents an alpha helical motif, and the light gray arrow represents a beta sheet motif.

### EXAMPLE 2

### Using a Targeting Peptide to Engineer New Metabolic Modules

One of the peptides of SEQ ID NOS:1-190 or a fragment thereof can be attached to the N-terminus or C-terminus (depending on where the peptide is natively found) or between domains of a protein to target that protein to shell proteins expressed in bacteria can be engineered, thus providing a new approach to designing and optimizing catalysis in solution. An example of using the CcmN peptide to target a fluorescent protein to the carboxysome in cyanobacteria is described (data not shown). A second example of the strategy for using the peptide to target a fluorescent protein to carboxysome shell proteins heterologously expressed in E. coli is also described (data not shown).

E. coli cultures (strain BL21 DE3) were transformed with a plasmid containing the gene for the cyanobacterial carboxysome shell protein CcmK2 from Synechococcus elongatus PCC7942 (YP_400438) and co-transformed with a plasmid containing the gene for the cyanobacterial carboxysome shell protein CcmK3 and a plasmid containing a gene for Green Fluorescent Protein conjugated to the conserved targeting peptide sequence from CcmN of S. elongatus PCC7942 (18 C-terminal residues VYGKEQFLRMRQSMFPDR (SEQ ID NO: 191) with a GSGSGSGS linker (SEQ ID NO: 193) separating the GFP and peptide sequence). Plasmids were under lac repressor control. The cell cultures were grown to log phase (OD 0.6) at 37° C. and induced at 18° C. with 0.4 mM IPTG to express the shell proteins and GFP-target peptide conjugate. Cells were harvested after overnight induction fixed, embedded, and section using standard electron microscopy techniques. Thin sections were imaged on a Tecnai 12 microscope. High protein density regions were observed in many of the cells (image not shown) which is presumably from the expression of the carboxysome shell protein. The thin sections for the co-transformed culture were subsequently incubated with rabbit α-GFP antibodies as the primary antibody, washed, and then incubated with goat α-rabbit antibodies conjugated with gold particles. The immunolabeled sections were imaged to observed the presence of gold particles in the protein dense regions of the cell to show localization of the presumably shell protein (CcmK3) induced cellular substructure and the GFP-peptide conjugate (image not shown).

This is a way of bringing groups of enzymes that are functionally related into an organism or into solution. By delivering the enzymes to be encapsulated in a shell protein module, it is possible to introduce new functions that might otherwise be toxic to the cell, or incompatible with other aspects of cellular metabolism. Based on the design principles of naturally occurring metabolic modules, the naturally occurring assemblies of interior components and shell, we will be able to deliver groups of enzymes that are already (partially) optimized with respect to intermolecular interactions.

For example, many of the naturally occurring types of BMCs (Table 1) encapsulate reactions that produce toxic or volatile intermediates or encapsulate enzymes that are oxygen sensitive (e.g. RuBisCO). Other oxygen sensitive enzymes (e.g. nitrogenase) could be encapsulated in a BMC by attachment of the targeting signal to that enzyme and optimizing shell selectivity for nitrogenase-related metabolite flow by site-directed mutagenesis and directed/adaptive evolution.

Expression of shell proteins to self assemble into molecular layers and then targeting enzymes to the molecular layers using the peptide provides another example of how the targeting peptide can be used to attach proteins to a scaffold. Co-localization of functionally related enzymes in space, on a layer of shell proteins, can be used to enhance the overall rate of a series of enzymatic reactions.

In a second example, enzymes known to be targeted to BMCs could be used as a scaffold for new catalytic functionality. B12-independent diol dehydratase (a BMC encapsulated enzyme) is a homolog of pyruvate formate lyase (an enzyme not known to be encapsulated into a BMC) which produces the valuable metabolite Acetyl CoA. Pyruvate formate lyase is oxygen sensitive. Because of the homology between pyruvate formate lyase and B12-independent diol dehydratase a small number amino substitutions could be used to convert B12-independent diol dehydratase into pyruvate formate lyase. Concomitant modification of the shell selectivity properties could be used to create pyruvate formate lyase-containing BMCs that could be expressed in anaerobic organisms to produce the valuable metabolite acetyl-CoA.

### EXAMPLE 3

### Using a Targeting Peptide to Engineer New Metabolic Modules

Syenchococcus elongatus PCC7942 was transformed with Yellow Fluorescent Protein (YFP) conjugated at the C-terminus to full-length CcmN (YP_400441) and under the native alphaphycocyanin promoter (papcA). The culture was grown under chloramphenicol selection at 30° C. in light. This was used as a positive control to show that carboxysome interior component CcmN is labeled with YFP. The image was captured at 100× magnification with a 3 second exposure time (YFP channel 513ex/530em) on a Zeiss AxioSkop 2 and was subsequently background subtracted using ImageJ software (Rasband, W. S., ImageJ, U.S. National Institutes of Health, Bethesda, Md., USA, http://rsb.info.nih.gov/ij/, 1997-2009.) The control indicates that CcmN is associated with the carboxysome gene cluster and contains the conserved peptide targeting sequence at its C-terminus.

A control experiment was then performed to show that CcmN and RuBisCO (RbcL) co-localize in a microcompartment. Synechococcus elogatus PCC7942 was co-transformed with a YFP-CcmN construct under the apcA promoter and the RuBisCO large subunit (RbcL) conjugated to Cyan Fluorescent Protein (CFP) at its C-terminus and under the ribosomal promoter prplC. The culture was grown under chloramphenicol and spectimnomycin selection at 30° C. in light. Images were captured at 100× magnification with a 3 second exposure time (513ex/530em) on a Zeiss Axioskop 2 and was subsequently background subtracted using ImageJ software, or at 100× magnification using a Applied Precision Deltavision Spectris DV4 deconvolution microscope. Each image was from the same z-plane taken at 500 ms exposure times using the YFP (513ex/530em) and CFP (433ex/475em) channels. The co-localization of fluorescence intensity provides a positive control for the localization of CcmN to the carboxysome since RbcL is known to localize to the carboxysome as well.

Syenchococcus elongatus PCC7942 was co-transformed with YFP conjugated with the linker region and the conserved targeting peptide from the C-terminus of CcmN [39 C-terminal residues from CcmN and identified as (132-VSSSEPAGRSPQSSAIAHPTKVYGKEQFLRMRQSMFPDR-160; SEQ ID NO:192)] and RbcL-CFP both under the rplC promoter. The culture was grown at 30° C. in light under chloramphenicol and spectinomycin selection. Images (not shown) were captured at 100× magnification with a 3 second exposure time (YFP channel 513ex/530em) on a Zeiss AxioSkop 2 and subsequently background subtracted using ImageJ software. Punctate fluorescence intensity was visible which is consistent with carboxysomal localization but the fluorescent signal was weak/undetectable in the CFP channel from the RbcL-CFP to provide conclusive evidence based on the co-localization of fluorescent signal.

In a second experiment, Syenchococcus elongatus PCC7942 was co-transformed with YFP conjugated to the linker region and conserved targeting peptide from the C-terminus of CcmN [39 C-terminal residues from CcmN (132-VSSSEPAGRSPQSSAIAHPTKVYGKEQFLRMRQSMFPDR-160; SEQ ID NO:192)] under the apcA promoter and RbcL-CFP under the rplC promoter. The culture was grown at 30° C. in light under chloramphenicol and spectinomycin selection. The images were captured at 100× magnification with a 3 second exposure time (YFP channel 513ex/530em) on a Zeiss AxioSkop 2 and subsequently background subtracted using ImageJ software. Again, punctate fluorescence intensity was visible which is consistent with carboxysomal localization but the fluorescent signal was weak/undetectable in the CFP channel from the RbcL-CFP to provide conclusive evidence based on the co-localization of fluorescent signal.

The above examples are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

What is claimed is:

1. A polynucleotide encoding a heterologous fusion protein targeted to a bacterial microcompartment, comprising:
a first nucleotide sequence that encodes a bacterial microcompartment targeting peptide;
a second nucleotide sequence that encodes a heterologous protein to be targeted to a bacterial microcompartment; and
a promoter sequence operably linked to the first nucleotide sequence and the second nucleotide sequence.

2. The polynucleotide of claim 1, wherein the first nucleotide sequence encodes a targeting peptide comprising an alpha helical secondary structure.

3. The polynucleotide of claim 2, wherein the first nucleotide sequence encodes a targeting peptide comprising an amphipathic alpha helical secondary structure.

4. The polynucleotide of claim 2, wherein the first nucleotide sequence encodes a targeting peptide comprising an alpha helical secondary structure having a four residue hydrophobic polar face.

5. The polynucleotide of claim 1, wherein the first nucleotide sequence and the second nucleotide sequence are joined to one another through a linker sequence that encodes a linker peptide.

6. The polynucleotide of claim 5, wherein the linker peptide comprises at least one amino acid selected from the group consisting of: proline, glycine, and alanine.

7. The polynucleotide of claim 1, wherein the first nucleotide sequence encodes a targeting peptide having the consensus sequence of SEQ ID NO: 45.

8. The polynucleotide of claim 1, wherein the promoter sequence is an inducible promoter sequence.

9. The polynucleotide of claim 1, wherein the polynucleotide comprises a marker gene that encodes for antibiotic resistance to a particular antibiotic.

10. The polynucleotide of claim 1, wherein the heterologous protein is an enzyme.

11. The polynucleotide of claim 10, wherein the enzyme is a metabolic enzyme.

12. An expression cassette for expressing a heterologous protein that binds to a bacterial micro compartment, comprising:
a promoter sequence;
a first nucleotide sequence operably linked to the promoter sequence and configured to encode a bacterial microcompartment targeting peptide;
a linker nucleotide sequence operably linked to the first nucleotide sequence;
a second nucleotide sequence operably linked to the linker sequence and configured to encode a heterologous protein to be targeted to a bacterial micro compartment.

13. The expression cassette of claim 12, further comprising at least one gene that encodes a microcompartment shell protein.

14. The polynucleotide of claim 12, wherein the first nucleotide sequence encodes a targeting peptide comprising an alpha helical secondary structure.

15. The polynucleotide of claim 14, wherein the first nucleotide sequence encodes a targeting peptide comprising an amphipathic alpha helical secondary structure.

16. The polynucleotide of claim 14, wherein the first nucleotide sequence encodes a targeting peptide comprising an alpha helical secondary structure having a four residue hydrophobic polar face.

17. The polynucleotide of claim 12, wherein the linker nucleotide sequence encodes at least one amino acid selected from the group consisting of: proline, glycine, and alanine.

18. The polynucleotide of claim 12, wherein the first nucleotide sequence encodes a targeting peptide having the consensus sequence of SEQ ID NO: 45.

19. A cell comprising in its genome at least one stably incorporated expression cassette of claim 1.

20. The cell of claim 19, wherein the cell is selected from the group consisting of a bacterial cell; plant cell, or eukaryotic cell.
